(19) United States
(12) Patent Application Publication
(10) Pub. No.: US 2017/0114359 A1
(43) Pub. Date: Apr. 27, 2017

(54) GENES AND USES FOR PLANT ENHANCEMENT

(71) Applicant: Monsanto Technology, LLC, St Louis, MO (US)

(72) Inventors: Maria C. Bohannon, Raleigh, NC (US); Marie Coffin, Cary, NC (US); Amanda Winslow, Belvidere, NC (US); Faten Shaikh, Cary, NC (US)

(21) Appl. No.: 15/330,419

(22) Filed: Sep. 19, 2016

Related U.S. Application Data
(63) Continuation of application No. 14/121,853, filed on Oct. 24, 2014, now abandoned.
Application No. 14/121,853 is a continuation of application No. 12/290,057, filed on Oct. 27, 2008, now abandoned.
(60) Provisional application No. 61/190,041, filed on Oct. 31, 2007.

(51) Int. Cl. C12N 15/82; A01H 1/04; C07K 14/415; C12Q 1/68

(52) U.S. Cl. CPC C12N 15/8275; C12N 15/8273; C12N 15/8261; C12N 15/8262; C12N 15/8247; C12N 15/8274; C12Q 1/6895; C12N 15/8241; A01H 1/04; C07K 14/415; C12Q 2600/13

(57) ABSTRACT

Transgenic seed for crops with enhanced agronomic traits are provided by trait-improving recombinant DNA in the nucleus of cells of the seed where plants grown from such transgenic seed exhibit one or more enhanced traits as compared to a control plant. Of particular interest are transgenic plants that have increased yield. The present invention also provides recombinant DNA molecules for expression of a protein, and recombinant DNA molecules for suppression of a protein.

### CROSS REFERENCE TO RELATED APPLICATIONS

This application is a continuation of and claims the benefit of priority to U.S. application Ser. No. 12/290,057, filed Oct. 27, 2008, which claims benefit of U.S. provisional application Ser. No. 61/190,041 filed on Oct. 30, 2007, which applications are incorporated herein by reference and made a part hereof in their entirety, and the benefit of priority of each of which is claimed herein.

### INCORPORATION OF SEQUENCE LISTING

Two copies of the sequence listing (Copy 1 and Copy 2), both on CD-R, each containing the file named 38-21(54975)B_seqlisting.txt, which is 12,914,688 bytes (measured in MS-WINDOWS) and was created on Sep. 7, 2016, are incorporated herein by reference in their entirety.

### INCORPORATION OF COMPUTER PROGRAM LISTING

A Computer Program Listing (Copy 1 and Copy 2) containing folders hmmer-2.3.2 and 47pfamDir, all on CD-Rs, are submitted herewith and are incorporated herein by reference in their entirety. Folder hmmer-2.3.2 contains the source code and other associated file for implementing the HMMer software for Pfam analysis. Folder 47pfamDir contains 47 profile Hidden Markov Models. Both folders were created on the disks on Sep. 7, 2016, having a total size of 4,816,896 bytes when measured in MS-WINDOWS® operating system.

### FIELD OF THE INVENTION

Disclosed herein are transgenic plant cells, plants and seeds comprising recombinant DNA and methods of making and using such plant cells, plants and seeds.

### SUMMARY OF THE INVENTION

This invention provides plant cell nuclei with recombinant DNA that imparts enhanced agronomic traits in transgenic plants having the nuclei in their cells, e.g. enhanced water use efficiency, enhanced cold tolerance, enhanced heat tolerance, enhanced shade tolerance, enhanced high salinity tolerance, increased yield, enhanced nitrogen use efficiency, enhanced seed protein or enhanced seed oil. In certain cases the trait is imparted by producing in the cells a protein that is encoded by recombinant DNA and/or in other cases the trait is imparted by suppressing the production of a protein that is natively produced in the cells.

Such recombinant DNA in a plant cell nucleus of this invention is provided in as a construct comprising a promoter that is functional in plant cells and that is operably linked to DNA that encodes a protein or to DNA that results in gene suppression. Such DNA in the construct is sometimes defined by protein domains of an encoded protein targeted for production or suppression, e.g. a “Pfam domain module” (as defined herein below) from the group of Pfam domain modules identified in Table 17. Alternatively, e.g. where a Pfam domain module is not available, such DNA in the construct is defined a consensus amino acid sequence of an encoded protein that is targeted for production or suppression, e.g. a protein having amino acid sequence with at least 90% identity to a consensus amino acid sequence in the group of SEQ ID NO: 4819 through SEQ ID NO: 4825.

Other aspects of the invention are directed to specific derivative physical forms of the transgenic plant cell nuclei, e.g. where such a transgenic nucleus is present in a transgenic plant cell, a transgenic plant including plant part(s) such as progeny transgenic seed, and a haploid reproductive derivative of plant cell such as transgenic pollen and transgenic ovule. Such plant cell nuclei and derivatives are advantageously selected from a population of transgenic plants regenerated from plant cells having a nucleus that is transformed with recombinant DNA by screening the transgenic plants or progeny seeds in the population for an enhanced trait as compared to control plants or seed that do not have the recombinant DNA in their nuclei, where the enhanced trait is enhanced water use efficiency, enhanced cold tolerance, enhanced heat tolerance, enhanced shade tolerance, enhanced high salinity tolerance, increased yield, enhanced nitrogen use efficiency, enhanced seed protein or enhanced seed oil.

In other aspects of the invention the nuclei of plant cells and derivative transgenic cells, plants, seeds, pollen and ovules further include recombinant DNA expressing a protein that provides tolerance from exposure to one or more herbicide applied at levels that are lethal to a wild type plant. Such herbicide. tolerance is not only an advantageous trait in such plants but is also useful as a selectable marker in the transformation methods for producing the nuclei and nuclei derivatives of the invention. Such herbicide tolerance includes tolerance to a glyphosate, dicamba, or glufosinate herbicide.

Yet other aspects of the invention provide transgenic plant cell nuclei which are homozygous for the recombinant DNA. The transgenic plant cell nuclei of the invention and derivative cells, plants, seed and haploid reproductive derivatives of the invention are advantageously provided in corn, soybean, cotton, canola, alfalfa, wheat, rice plants, or combinations thereof.

This invention also provides methods for manufacturing non-natural, transgenic seed that can be used to produce a crop of transgenic plants with an enhanced trait resulting from expression of stably-integrated, recombinant DNA in the nucleus of the plant cells. More specifically the method includes, but are not limited to, (a) screening a population of plants for an enhanced trait and recombinant DNA, where individual plants in the population can exhibit the trait at a level less than, essentially the same as or greater than the level that the trait is exhibited in control plants which do not express the recombinant DNA; (b) selecting from the population one or more plants that exhibit the trait at a level greater than the level that said trait is exhibited in control plants and (c) collecting seed from a selected plant. Such method can further include the steps of (a) verifying that the recombinant DNA is stably integrated in said selected plants; and (b) analyzing tissue of a selected plant to determine the production of a protein having the function of a protein encoded by a recombinant DNA with a sequence of one of SEQ ID NO: 1-114; In one aspect of the invention the plants in the population can further include DNA expressing a protein that provides tolerance to exposure to an herbicide applied at levels that are lethal to wild type plant cells and where the selecting is effected by treating the population with the herbicide, e.g. a glyphosate, dicamba, or glufosinate compound. In another aspect of the invention, the plants are selected by identifying plants with the enhanced trait. The methods can be used for manufacturing corn, soybean, cotton, canola, alfalfa, wheat and/or rice seed selected as having one of the enhanced traits described above.

Another aspect of the invention provides a method of producing hybrid corn seed including the step of acquiring hybrid corn seed from a herbicide tolerant corn plant which also has a nucleus of this invention with stably-integrated, recombinant DNA. The method can further include the steps of producing corn plants from said hybrid corn seed, where a fraction of the plants produced from said hybrid corn seed is homozygous for said recombinant DNA, a fraction of the plants produced from said hybrid corn seed is hemizygous for said recombinant DNA, and/or a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

### BRIEF DESCRIPTION OF THE DRAWINGS

FIGS. 1, 2 and 3 are pictures illustrating plasmid maps.

FIG. 4 illustrates a consensus amino acid sequence of SEQ ID NO: 181 and its homologs.

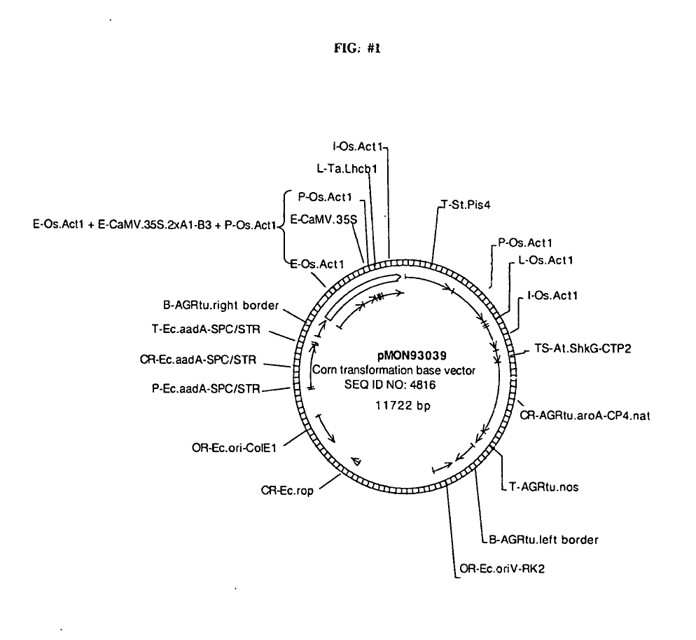

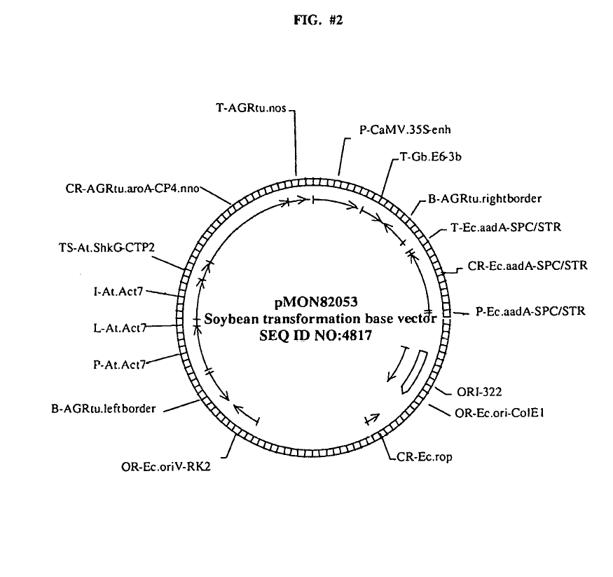

### DETAILED DESCRIPTION OF THE INVENTION

In the attached sequence listing:

SEQ ID NO: 1-114 are nucleotide sequences of the coding strand of DNA for “genes” used in the recombinant DNA imparting an enhanced trait in plant cells, i.e. each represents a coding sequence for a protein;

SEQ ID NO: 115-228 are amino acid sequences of the cognate protein of the “genes” with nucleotide coding sequence 1-114;

SEQ ID NO: 229-4815 are amino acid sequences of homologous proteins; SEQ ID NO: 4816 is a nucleotide sequence of a plasmid base vector useful for corn transformation; and

SEQ ID NO: 4817 is a DNA sequence of a plasmid base vector useful for soybean transformation.

SEQ ID NO: 4818 is a DNA sequence of a plasmid base vector useful for cotton transformation.

SEQ ID NO: 4819-4825 are consensus sequences.

Table 1 lists the protein SEQ ID NOs and their corresponding consensus SEQ ID NOs.

TABLE 1

| GENE ID | PEP SEQ ID | CONSENSUS SEQ ID |
|---|---|---|
| CGPG3454 | 151 | 4819 |
| CGPG4528 | 181 | 4820 |
| CGPG4560 | 183 | 4821 |
| CGPG7500 | 206 | 4822 |
| CGPG7639 | 213 | 4823 |
| CGPG8137 | 226 | 4824 |
| CGPG8180 | 228 | 4825 |

The nuclei of this invention are identified by screening transgenic plants for one or more traits including enhanced drought stress tolerance, enhanced heat stress tolerance, enhanced cold stress tolerance, enhanced high salinity stress tolerance, enhanced low nitrogen availability stress tolerance, enhanced shade stress tolerance, enhanced plant growth and development at the stages of seed imbibition through early vegetative phase, and enhanced plant growth and development at the stages of leaf development, flower production and seed maturity.

“Gene” means a chromosomal element for expressing a protein and specifically includes the DNA encoding a protein. In cases where expression of a target protein is desired, the pertinent part of a gene is the DNA encoding the target protein; in cases where suppression of a target is desired, the pertinent part of a gene is that part that is transcribed as mRNA. “Recombinant DNA” means a polynucleotide having a genetically engineered modification introduced through combination of endogenous and/or exogenous elements in a transcription unit. Recombinant DNA can include DNA segments obtained from different sources, or DNA segments obtained from the same source, but which have been manipulated to join DNA segments which do not naturally exist in the joined form.

“Trait” means a physiological, morphological, biochemical, or physical characteristic of a plant or particular plant material or cell, or any combinations thereof.

A “control plant” is a plant without trait-improving recombinant DNA in its nucleus. A control plant is used to measure and compare trait enhancement in a transgenic plant with such trait-improving recombinant DNA. A suitable control plant can be a non-transgenic plant of the parental line used to generate a transgenic plant herein. Alternatively, a control plant can be a transgenic plant having an empty vector or marker gene, but does not contain the recombinant DNA that produces the trait enhancement. A control plant can also be a negative segregant progeny of hemizygous transgenic plant. In certain demonstrations of trait enhancement, the use of a limited number of control plants can cause a wide variation in the control dataset. To minimize the effect of the variation within the control dataset, a “reference” is used. As use herein a “reference” is a trimmed mean of all data from both transgenic and control plants grown under the same conditions and at the same developmental stage. The trimmed mean is calculated by eliminating a specific percentage, i.e., 20%, of the smallest and largest observation from the data set and then calculating the average of the remaining observation.

“Trait enhancement” means a detectable and desirable difference in a characteristic in a transgenic plant relative to a control plant or a reference. In some cases, the trait enhancement can be measured quantitatively. For example, the trait enhancement can entail at least a 2% desirable difference in an observed trait, at least a 5% desirable difference, at least about a 10% desirable difference, at least about a 20% desirable difference, at least about a 30% desirable difference, at least about a 50% desirable difference, at least about a 70% desirable difference, or at least about a 100% difference, or an even greater desirable difference. In other cases, the trait enhancement is only measured qualitatively. It is known that there can be a natural variation in a trait. Therefore, the trait enhancement observed entails a change of the normal distribution of the trait in the transgenic plant compared with the trait distribution observed in a control plant or a reference, which is evaluated by statistical methods provided herein. Trait enhancement includes, but is not limited to, yield increase, including increased yield under non-stress conditions and increased yield under environmental stress conditions. Stress conditions can include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability, high plant density, or any combinations thereof.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, juvenile traits, or any combinations thereof. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that can confer an increase in overall plant yield. Such properties include enhanced plant morphology, plant physiology or improved components of the mature seed harvested from the transgenic plant.

“Yield-limiting environment” means the condition under which a plant would have the limitation on yield including environmental stress conditions.

“Stress condition” means a condition unfavorable for a plant, which adversely affect plant metabolism, growth and/or development. A plant under the stress condition typically shows reduced germination rate, retarded growth and development, reduced photosynthesis rate, and eventually leading to reduction in yield. Specifically, “water deficit stress” used herein refers to the sub-optimal conditions for water and humidity needed for normal growth of natural plants. Relative water content (RWC) can be used as a physiological measure of plant water deficit. It measures the effect of osmotic adjustment in plant water status, when a plant is under stressed conditions. Conditions which can result in water deficit stress include, but are not limited to, heat, drought, high salinity and PEG induced osmotic stress.

“Cold stress” means the exposure of a plant to a temperatures below (two or more degrees Celsius below) those normal for a particular species or particular strain of plant.

“Nitrogen nutrient” means any one or any mix of the nitrate salts commonly used as plant nitrogen fertilizer, including, but not limited to, potassium nitrate, calcium nitrate, sodium nitrate, ammonium nitrate. The term ammonium as used herein means any one or any mix of the ammonium salts commonly used as plant nitrogen fertilizer, e.g., ammonium nitrate, ammonium chloride, ammonium sulfate, etc.

“Low nitrogen availability stress” means a plant growth condition that does not contain sufficient nitrogen nutrient to maintain a healthy plant growth and/or for a plant to reach its typical yield under a sufficient nitrogen growth condition. For example, a limiting nitrogen condition can refers to a growth condition with 50% or less of the conventional nitrogen inputs. “Sufficient nitrogen growth condition” means a growth condition where the soil or growth medium contains or receives optimal amounts of nitrogen nutrient to sustain a healthy plant growth and/or for a plant to reach its typical yield for a particular plant species or a particular strain. One skilled in the art would recognize what constitute such soil, media and fertilizer inputs for most plant species.

“Shade stress” means a growth condition that has limited light availability that triggers the shade avoidance response in plant. Plants are subject to shade stress when localized at lower part of the canopy, or in close proximity of neighboring vegetation. Shade stress can become exacerbated when the planting density exceeds the average prevailing density for a particular plant species.

“Increased yield” of a transgenic plant of the present invention is evidenced and measured in a number of ways, including test weight, seed number per plant, seed weight, seed number per unit area (i.e., seeds, or weight of seeds, per acre), bushels per acre, tons per acre, tons per acre, kilo per hectare. For example, maize yield can be measured as production of shelled corn kernels per unit of production area, e.g., in bushels per acre or metric tons per hectare, often reported on a moisture adjusted basis, e.g., at 15.5% moisture. Increased yield can result from enhanced utilization of key biochemical compounds, such as nitrogen, phosphorous and carbohydrate, or from enhanced tolerance to environmental stresses, such as cold, heat, drought, salt, and attack by pests or pathogens. Trait-improving recombinant DNA can also be used to provide transgenic plants having enhanced growth and development, and ultimately increased yield, as the result of modified expression of plant growth regulators or modification of cell cycle or photosynthesis pathways.

A “plant promoter” is a promoter capable of initiating transcription in plant cells whether or not its origin is a plant cell. Exemplary plant promoters include, but are not limited to, those that are obtained from plants, plant viruses, and bacteria which include genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that can effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein, “operably linked” refers to the association of two or more nucleic acid elements in a recombinant DNA construct, e.g. as when a promoter is operably linked with DNA that is transcribed to RNA whether for expressing or suppressing a protein. Recombinant DNA constructs can be designed to express a protein which can be an endogenous protein, an exogenous homologue of an endogenous protein or an exogenous protein with no native homologue. Alternatively, recombinant DNA constructs can be designed to suppress the level of an endogenous protein, e.g. by suppression of the native gene. Such gene suppression can be effectively employed through a native RNA interference (RNAi) mechanism in which recombinant DNA comprises both sense and anti-sense oriented DNA matched to the gene targeted for suppression where the recombinant DNA is transcribed into RNA that can form a double-strand to initiate an RNAi mechanism. Gene suppression can also be effected by recombinant DNA that comprises anti-sense oriented DNA matched to the gene targeted for suppression. Gene suppression can also be effected by recombinant DNA that comprises DNA that is transcribed to a microRNA matched to the gene targeted for suppression. In the examples illustrating the invention recombinant DNA for effecting gene suppression that imparts is identified by the term “antisense”. It will be understood by a person of ordinary skill in the art that any of the ways of effecting gene suppression are contemplated and enabled by a showing of one approach to gene suppression.

A “consensus amino acid sequence” means an artificial, amino acid sequence indicating conserved amino acids in the sequence of homologous proteins as determined by statistical analysis of an optimal alignment, e.g. CLUSTALW, of amino acid sequence of homolog proteins. The consensus sequences listed in the sequence listing were created by identifying the most frequent amino acid at each position in a set of aligned protein sequences. When there was 100% identity in an alignment the amino acid is indicated by a capital letter. When the occurrence of an amino acid is at least about 70% in an alignment, the amino acid is indicated by a lower case letter. When there is no amino acid occurrence of at least about 70%, e.g. due to diversity or gaps, the amino acid is indicated by an “x”. When used to defined embodiments of the invention, a consensus amino acid sequence will be aligned with a query protein amino acid sequence in an optimal alignment, e.g. CLUSTALW. An embodiment of the invention will have identity to the conserved amino acids indicated in the consensus amino acid sequence.

As used herein a “homolog” means a protein in a group of proteins that perform the same biological function, e.g. proteins that belong to the same Pfam protein family and that provide a common enhanced trait in transgenic plants of this invention. Homologs are expressed by homologous genes. With reference to homologous genes, homologs include orthologs, i.e. genes expressed in different species that evolved from a common ancestral genes by speciation and encode proteins retain the same function, but do not include paralogs, i.e. genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have typically at least about 60% identity, in some instances at least about 70%, for example about 80% and even at least about 90% identity over the full length of a protein identified as being associated with imparting an enhanced trait when expressed in plant cells. In one aspect of the invention homolog proteins have an amino acid sequence that has at least 90% identity to a consensus amino acid sequence of proteins and homologs disclosed herein.

Homologs are identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

As used herein, “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100. Such optimal alignment is understood to be deemed as local alignment of DNA sequences. For protein alignment, a local alignment of protein sequences should allow introduction of gaps to achieve optimal alignment. Percent identity is calculated over the aligned length not including the gaps introduced by the alignment per se.

Homologous genes are genes which encode proteins with the same or similar biological function to the protein encoded by the second gene. Homologous genes can be generated by the event of speciation (see ortholog) or by the event of genetic duplication (see paralog). “Orthologs” refer to a set of homologous genes in different species that evolved from a common ancestral gene by specification. Normally, orthologs retain the same function in the course of evolution; and “paralogs” refer to a set of homologous genes in the same species that have diverged from each other as a consequence of genetic duplication. Thus, homologous genes can be from the same or a different organism. As used herein, “homolog” means a protein that performs the same biological function as a second protein including those identified by sequence identity search.

“Arabidopsis” means plants of Arabidopsis thaliana.

“Pfam” database is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

A “Pfam domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pfam domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected.

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software, a current version of which is provided in the appended computer listing. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are also included in the appended computer listing.

Version 19.0 of the HMMER software and Pfam databases were used to identify known domains in the proteins corresponding to amino acid sequence of SEQ ID NO: 115 through SEQ ID NO:228. All DNA encoding proteins that have scores higher than the gathering cutoff disclosed in Table 19 by Pfam analysis disclosed herein can be used in recombinant DNA of the plant cells of this invention, e.g. for selecting transgenic plants having enhanced agronomic traits. The relevant Pfam modules for use in this invention, as more specifically disclosed below, are Saccharop_dh, Isoamylase_{—} AP2, zf-C2H2, PLATZ, F-box::Tub, zf-C3HC4::YDG_SRA::zf-C3HC4,SBP, HLH, AP2, zf-B_box::zf-B_box, zf-C3HC4, AP2, HMG_box::HMG_box::1HMG_box, zf-C3HC4, zf-C2H2, GATA, HLH, AP2, NAM, zf-Dof, WRKY, AP2, HMG_box, zf-CCCH::KH_1::zf-CCCH, SRF-TF, WRKY, zf-C3HC4, zf-Dof, zf-Dof, AP2, AP2, DUF248, zf-C2H2, SRF-TF::K-box, zf-Dof, zf-C2H2, Myb_DNA-binding::Myb_DNA-binding, Myb_DNA-binding::Myb_DNA-binding::Myb_DNA-binding, SRF-TF, Pex2_Pex12::zf-C3HC4, bZIP_2, HLH, GRAS, Myb_DNA-binding, GRAS, F-box, GRAS, WRKY, AT_hook::DUF296, GRAS, Myb_DNA-binding::Myb_DNA-binding, Myb_DNA-binding::Myb_DNA-binding, HLH, zf-C21-12, NAM, zf-B_box::zf-B_box, Myb_DNA-binding, NAM, PHD, SRF-TF::K-box, zf-C3HC4, HLH, SRF-TF::K-box, Myb_DNA-binding, WRKY, SRF-TF::K-box, Myb_DNA-binding, Myb_DNA-binding::Linker_histone, Myb_DNA-binding::Myb_DNA-binding, Myb_DNA-binding::Myb_DNA-binding, bZIP_1, Myb_DNA-binding::Myb_DNA-binding, SRF-TF::K-box, SRF-TF::K-box, CBFD_NFYB_HMF, AUX_IAA, Myb_DNA-binding::Myb_DNA-binding, LIM::LIM, IBR, SET, bZIP_1, Mov34, ZZ::Myb_DNA-binding::SWIRM, bZIP_1, E2F_TDP::E2F_TDP, Myb_DNA-binding, Prefoldin, NAM, HD-ZIP_N::Homeobox::HALZ, Myb_DNA-binding::Myb_DNA-binding, AP2, GATA, zf-C3HC4, SRF-TF::K-box, bZIP_1, TCP, zf-C3HC4, Ank::Ank::zf-CCCH::zf-CCCH, Myb_DNA-binding::Myb_DNA-binding, TCP, AP2, ZZ, zf-C2H2::zf-C2H2, AP2, SRF-TF::K-box, B3, zf-LSD1::zf-LSD1, and HLH.

### Recombinant DNA Constructs

The invention uses recombinant DNA for imparting one or more enhanced traits to transgenic plant when incorporated into the nucleus of the plant cells. Such recombinant DNA is a construct comprising a promoter operatively linked to to DNA for expression or suppression of a target protein in plant cells. Other construct components can include additional regulatory elements, such as 5′ or 3′ untranslated regions (such as polyadenylation sites), intron regions, and transit or signal peptides. Such recombinant DNA constructs can be assembled using methods known to those of ordinary skill in the art.

Recombinant constructs prepared in accordance with the present invention also generally include a 3′ untranslated DNA region (UTR) that typically contains a polyadenylation sequence following the polynucleotide coding region. Examples of useful 3′ UTRs include those from the nopaline synthase gene of Agrobacterium tumefaciens (nos), a gene encoding the small subunit of a ribulose-1,5-bisphosphate carboxylase-oxygenase (rbcS), and the T7 transcript of Agrobacterium tumefaciens.

Constructs and vectors can also include a transit peptide for targeting of a gene target to a plant organelle, particularly to a chloroplast, leucoplast or other plastid organelle. For descriptions of the use of chloroplast transit peptides, see U.S. Pat. No. 5,188,642 and U.S. Pat. No. 5,728,925, incorporated herein by reference.

Table 2 provides a list of genes that provided recombinant DNA that was expressed in a model plant and identified from screening as imparting an enhanced trait. When the stated orientation is “sense”, the expression of the gene or a homolog in a crop plant provides the means to identify transgenic events that provide an enhanced trait in the crop plant. When the stated orientation is “antisense”, the suppression of the native homolog in a crop plant provides the means to identify transgenic events that provide an enhanced trait in the crop plant. In some cases the expression/suppression in the model plant exhibited an enhanced trait that corresponds to an enhanced agronomic trait, e.g. cold stress tolerance, water deficit stress tolerance, low nitrogen stress tolerance and the like. In other cases the expression/suppression in the model plant exhibited an enhanced trait that is a surrogate to an enhanced agronomic trait, e.g. salinity stress tolerance being a surrogate to drought tolerance or improvement in plant growth and development being a surrogate to enhanced yield. Even when expression of a transgene or suppression of a native gene imparts an enhanced trait in a model plant, not every crop plant expressing the same transgene or suppressing the same native gene will necessarily demonstrate an indicated enhanced agronomic trait. For instance, it is well known that multiple transgenic events are required to identify a transgenic plant that can exhibit an enhanced agronomic trait. A skilled artisan can identify a transgenic plant cell nuclei, cell, plant or seed by making number of transgenic events, typically a very large number, and engaging in screening processes identified in this specification and illustrated in the examples. For example, a screening process includes selecting only those transgenic events with an intact, single copy of the recombinant DNA in a single locus of the host plant genome and further screening for transgenic events that impart a desired trait that is replicatable when the recombinant DNA is introgressed into a variety of germplams without imparting significant adverse traits.

An understanding of Table 2 is facilitated by the following description of the headings:

“NUC SEQ ID NO” refers to a SEQ ID NO. for particular DNA sequence in the Sequence Listing.

“PEP SEQ ID NO” refers to a SEQ ID NO. in the Sequence Listing for the amino acid sequence of a protein cognate to a particular DNA

“construct_id” refers to an arbitrary number used to identify a particular recombinant DNA construct comprising the particular DNA.

“Gene ID” refers to an arbitrary name used to identify the particular DNA.

“orientation” refers to the orientation of the particular DNA in a recombinant DNA construct relative to the promoter.

TABLE 2

| NUC | PEP | | | |
|---|---|---|---|---|
| Seq | SEQ | | Construct | |
| ID No. | ID | Gene ID | ID | Orientation |
| 1 | 115 | CGPG110 | 10177 | ANTI-SENSE |
| 2 | 116 | CGPG1135 | 12155 | ANTI-SENSE |
| 3 | 117 | CGPG1180 | 12233 | SENSE |
| 4 | 118 | CGPG1197 | 11873 | ANTI-SENSE |
| 5 | 119 | CGPG1802 | 17308 | SENSE |
| 6 | 120 | CGPG2561 | 78706 | SENSE |
| 7 | 121 | CGPG2580 | 72666 | SENSE |
| 8 | 122 | CGPG2582 | 17520 | SENSE |
| 9 | 123 | CGPG2591 | 72672 | SENSE |
| 10 | 124 | CGPG2597 | 78977 | SENSE |
| 11 | 125 | CGPG2602 | 17468 | SENSE |
| 12 | 126 | CGPG2642 | 75090 | SENSE |
| 13 | 127 | CGPG2651 | 72018 | SENSE |
| 14 | 128 | CGPG2662 | 17524 | SENSE |
| 15 | 129 | CGPG2708 | 72078 | SENSE |
| 16 | 130 | CGPG2710 | 72977 | SENSE |
| 17 | 131 | CGPG2722 | 17445 | SENSE |
| 18 | 132 | CGPG2736 | 78979 | SENSE |
| 19 | 133 | CGPG2759 | 18453 | SENSE |
| 20 | 134 | CGPG2765 | 17913 | SENSE |
| 21 | 135 | CGPG2802 | 18210 | SENSE |
| 22 | 136 | CGPG2823 | 78710 | SENSE |
| 23 | 137 | CGPG2889 | 73606 | SENSE |
| 24 | 138 | CGPG2902 | 18446 | SENSE |
| 25 | 139 | CGPG2913 | 75011 | SENSE |
| 26 | 140 | CGPG2945 | 18507 | SENSE |
| 27 | 141 | CGPG2962 | 18388 | SENSE |
| 28 | 142 | CGPG2967 | 18540 | SENSE |
| 29 | 143 | CGPG2983 | 19653 | SENSE |
| 30 | 144 | CGPG2996 | 18411 | SENSE |
| 31 | 145 | CGPG3307 | 70737 | SENSE |
| 32 | 146 | CGPG3311 | 18315 | SENSE |
| 33 | 147 | CGPG3350 | 75080 | SENSE |
| 34 | 148 | CGPG3355 | 19191 | SENSE |
| 35 | 149 | CGPG3447 | 18431 | SENSE |
| 36 | 150 | CGPG3453 | 18433 | SENSE |
| 37 | 151 | CGPG3454 | 18434 | SENSE |
| 38 | 152 | CGPG3455 | 19536 | SENSE |
| 39 | 153 | CGPG3488 | 76577 | SENSE |
| 40 | 154 | CGPG353 | 11113 | ANTI-SENSE |
| 41 | 155 | CGPG3537 | 19614 | SENSE |
| 42 | 156 | CGPG355 | 18204 | SENSE |
| 43 | 157 | CGPG3751 | 73614 | SENSE |
| 44 | 158 | CGPG3755 | 70450 | SENSE |
| 45 | 159 | CGPG3756 | 70618 | SENSE |
| 46 | 160 | CGPG3757 | 70458 | SENSE |
| 47 | 161 | CGPG3776 | 71306 | SENSE |
| 48 | 162 | CGPG3778 | 70451 | SENSE |
| 49 | 163 | CGPG3799 | 70453 | SENSE |
| 50 | 164 | CGPG3822 | 71311 | SENSE |
| 51 | 165 | CGPG3890 | 76533 | SENSE |
| 52 | 166 | CGPG3965 | 70955 | SENSE |
| 53 | 167 | CGPG3966 | 77708 | SENSE |
| 54 | 168 | CGPG3974 | 70954 | SENSE |
| 55 | 169 | CGPG3977 | 19742 | SENSE |
| 56 | 170 | CGPG3984 | 70960 | SENSE |
| 57 | 171 | CGPG3988 | 19940 | SENSE |
| 58 | 172 | CGPG4068 | 70921 | SENSE |
| 59 | 173 | CGPG4105 | 70953 | SENSE |
| 60 | 174 | CGPG4109 | 70978 | SENSE |
| 61 | 175 | CGPG4125 | 70940 | SENSE |
| 62 | 176 | CGPG4126 | 70977 | SENSE |
| 63 | 177 | CGPG4167 | 70993 | SENSE |
| 64 | 178 | CGPG4176 | 19762 | SENSE |
| 65 | 179 | CGPG4193 | 70938 | SENSE |
| 66 | 180 | CGPG4210 | 78653 | SENSE |
| 67 | 181 | CGPG4528 | 73216 | SENSE |
| 68 | 182 | CGPG4540 | 71139 | SENSE |
| 69 | 183 | CGPG4560 | 74227 | SENSE |
| 70 | 184 | CGPG4571 | 70678 | SENSE |
| 71 | 185 | CGPG4622 | 70769 | SENSE |
| 72 | 186 | CGPG4700 | 71643 | SENSE |
| 73 | 187 | CGPG475 | 70242 | SENSE |
| 74 | 188 | CGPG478 | 12325 | ANTI-SENSE |
| 75 | 189 | CGPG497 | 70356 | SENSE |
| 76 | 190 | CGPG5283 | 72034 | SENSE |
| 77 | 191 | CGPG5284 | 72046 | SENSE |
| 78 | 192 | CGPG5294 | 72071 | SENSE |
| 79 | 193 | CGPG5308 | 72116 | SENSE |
| 80 | 194 | CGPG5322 | 72108 | SENSE |
| 81 | 195 | CGPG5427 | 77605 | SENSE |
| 82 | 196 | CGPG5595 | 73978 | SENSE |
| 83 | 197 | CGPG5806 | 73032 | SENSE |
| 84 | 198 | CGPG626 | 11157 | SENSE |
| 85 | 199 | CGPG6434 | 73493 | SENSE |
| 86 | 200 | CGPG688 | 71228 | ANTI-SENSE |
| 87 | 201 | CGPG7334 | 74889 | SENSE |
| 88 | 202 | CGPG7339 | 74854 | SENSE |
| 89 | 203 | CGPG7350 | 74891 | SENSE |
| 90 | 204 | CGPG7475 | 75314 | SENSE |
| 91 | 205 | CGPG7493 | 75340 | SENSE |
| 92 | 206 | CGPG7500 | 75329 | SENSE |
| 93 | 207 | CGPG7598 | 75453 | SENSE |
| 94 | 208 | CGPG7600 | 75477 | SENSE |
| 95 | 209 | CGPG7604 | 75430 | SENSE |
| 96 | 210 | CGPG7615 | 75467 | SENSE |
| 97 | 211 | CGPG7630 | 75457 | SENSE |
| 98 | 212 | CGPG7631 | 75469 | SENSE |
| 99 | 213 | CGPG7639 | 75470 | SENSE |
| 100 | 214 | CGPG7644 | 75435 | SENSE |
| 101 | 215 | CGPG7691 | 77822 | SENSE |
| 102 | 216 | CGPG7696 | 75505 | SENSE |
| 103 | 217 | CGPG7703 | 75589 | SENSE |
| 104 | 218 | CGPG7707 | 75542 | SENSE |
| 105 | 219 | CGPG7731 | 75545 | SENSE |
| 106 | 220 | CGPG7734 | 75581 | SENSE |
| 107 | 221 | CGPG7753 | 75524 | SENSE |
| 108 | 222 | CGPG7836 | 75658 | SENSE |
| 109 | 223 | CGPG7867 | 75738 | SENSE |
| 110 | 224 | CGPG8105 | 77356 | SENSE |
| 111 | 225 | CGPG8132 | 77362 | SENSE |
| 112 | 226 | CGPG8137 | 77363 | SENSE |
| 113 | 227 | CGPG8175 | 77371 | SENSE |
| 114 | 228 | CGPG8180 | 77941 | SENSE |

### Recombinant DNA

DNA for use in the present invention to improve traits in plants have a nucleotide sequence of SEQ ID NO:1 through SEQ ID NO:114, as well as the homologs of such DNA molecules. A subset of the DNA for gene suppression aspects of the invention includes fragments of the disclosed full polynucleotides consisting of oligonucleotides of 21 or more consecutive nucleotides. Oligonucleotides the larger molecules having a sequence selected from the group consisting of SEQ ID NO: 1 through SEQ ID NO: 114 are useful as probes and primers for detection of the polynucleotides used in the invention. Also useful in this invention are variants of the DNA. Such variants can be naturally occurring, including DNA from homologous genes from the same or a different species, or can be non-natural variants, for example DNA synthesized using chemical synthesis methods, or generated using recombinant DNA techniques. Degeneracy of the genetic code provides the possibility to substitute at least one base of the protein encoding sequence of a gene with a different base without causing the amino acid sequence of the polypeptide produced from the gene to be changed. Hence, a DNA useful in the present invention can have any base sequence that has been changed from the sequences provided herein by substitution in accordance with degeneracy of the genetic code.

Homologs of the genes providing DNA demonstrated as useful in improving traits in model plants disclosed herein will generally have significant identity with the DNA disclosed herein. DNA is substantially identical to a reference DNA if, when the sequences of the polynucleotides are optimally aligned there is at least about 60% nucleotide equivalence over a comparison window. The DNA can also be about 70% equivalence, about 80% equivalence; about 85% equivalence; about 90%; about 95%; or even about 98% or 99% equivalence over a comparison window. A comparison window is at least about 50-100 nucleotides, and/or is the entire length of the polynucleotide provided herein. Optimal alignment of sequences for aligning a comparison window can be conducted by algorithms or by computerized implementations of these algorithms (for example, the Wisconsin Genetics Software Package Release 7.0-10.0, Genetics Computer Group, 575 Science Dr., Madison, Wis.). The reference polynucleotide can be a full-length molecule or a portion of a longer molecule. In one embodiment, the window of comparison for determining polynucleotide identity of protein encoding sequences is the entire coding region.

Proteins useful for imparting enhanced traits are entire proteins or at least a sufficient portion of the entire protein to impart the relevant biological activity of the protein. Proteins used for generation of transgenic plants having enhanced traits include the proteins with an amino acid sequence provided herein as SEQ ID NO: 115 through SEQ ID NO: 228, as well as homologs of such proteins.

Homologs of the trait-improving proteins provided herein generally demonstrate significant sequence identity. Of particular interest are proteins having at least about 50% sequence identity, at least about 70% sequence identity or higher, e.g., at least about 80% sequence identity with an amino acid sequence of SEQ ID NO:115 through SEQ ID NO: 228. Useful proteins also include those with higher identity, e.g., at lease about 90% to at least about 99% identity. Identity of protein homologs is determined by aligning the amino acid sequence of a putative protein homolog with a defined amino acid sequence and by calculating the percentage of identical and conservatively substituted amino acids over the window of comparison. The window of comparison for determining identity can be the entire amino acid sequence disclosed herein, e.g., the full sequence of any of SEQ ID NO: 115 through SEQ ID NO: 228.

The relationship of homologs with amino acid sequences of SEQ ID NO: 229 to SEQ ID NO: 4813 to the proteins with amino acid sequences of SEQ ID NO: to 115 to SEQ ID NO: 228 are found in the listing of Table 16.

Other functional homolog proteins differ in one or more amino acids from those of a trait-improving protein disclosed herein as the result of one or more of the well-known conservative amino acid substitutions, e.g., valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention includes proteins that differ in one or more amino acids from those of a described protein sequence as the result of deletion or insertion of one or more amino acids in a native sequence.

Genes that arc homologous to each other can be grouped into families and included in multiple sequence alignments. Then a consensus sequence for each group can be derived. This analysis enables the derivation of conserved and class-(family) specific residues or motifs that are functionally important. These conserved residues and motifs can be further validated with 3D protein structure if available. The consensus sequence can be used to define the full scope of the invention, e.g., to identify proteins with a homolog relationship. Thus, the present invention contemplates that protein homologs include proteins with an amino acid sequence that has at least 90% identity to such a consensus amino acid sequence sequences.

### Promoters

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens, caulimovirus promoters such as the cauliflower mosaic virus or Figwort mosaic virus promoters. For instance, see U.S. Pat. Nos. 5,858,742 and 5,322,938 which disclose versions of the constitutive promoter derived from cauliflower mosaic virus (CaMV35S), U.S. Pat. No. 5,378,619 which discloses a Figwort Mosaic Virus (FMV) 35S promoter, U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, and U.S. patent application Ser. No. 10/739,565 which discloses water-deficit inducible promoters, all of which are incorporated herein by reference. These and numerous other promoters that function in plant cells are known to those skilled in the art and available for use in recombinant polynucleotides of the present invention to provide for expression of desired genes in transgenic plant cells.

Furthermore, the promoters can include multiple “enhancer sequences” to assist in elevating gene expression. Such enhancers are known in the art. By including an enhancer sequence with such constructs, the expression of the selected protein can be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin I and rice actin 2 genes. Examples of other enhancers that can be used in accordance with the invention include elements from the CaMV 35S promoter, octopine synthase genes, the maize alcohol dehydrogenase gene, the maize shrunken 1 gene and promoters from non-plant eukaryotes.

In some aspects of the invention, the promoter element in the DNA construct can be capable of causing sufficient expression to result in the production of an effective amount of a polypeptide in water deficit conditions. Such promoters can be identified and isolated from the regulatory region of plant genes that are over expressed in water deficit conditions. Specific water-deficit-inducible promoters for use in this invention are derived from the 5′ regulatory region of genes identified as a heat shock protein 17.5 gene (HSP17.5), an HVA22 gene (HVA22), a Rab17 gene and a cinnamic acid 4-hydroxylase (CA4H) gene (CA4H) of Zea maize. Such water-deficit-inducible promoters are disclosed in U.S. application Ser. No. 10/739,565, incorporated herein by reference.

In some aspects of the invention, sufficient expression in plant seed tissues is desired to effect improvements in seed composition. Exemplary promoters for use for seed composition modification include promoters from seed genes such as napin (U.S. Pat. No. 5,420,034), maize L3 olcosin (U.S. Pat. No. 6,433,252), zein Z27 (Russell et al., (1997) Transgenic Res. 6(2):157-166), globulin 1 (Belanger et al., (1991) Genetics 129:863-872), glutelin 1 (Russell (1997) supra), and peroxiredoxin antioxidant (Perl) (Stacy et al., (1996) Plant Mol Biol. 31(6):1205-1216).

In some aspects of the invention, expression in plant green tissues is desired. Promoters of interest for such uses include those from genes such as SSU (Fischhoff, et al., (1992) Plant Mol Biol. 20:81-93), aldolase and pyruvate orthophosphate dikinase (PPDK) (Taniguchi, et al., (2000) Plant Cell Physiol. 41(1):42-48).

Gene suppression includes any of the well-known methods for suppressing transcription of a gene or the accumulation of the mRNA corresponding to that gene thereby preventing translation of the transcript into protein. Posttranscriptional gene suppression is mediated by transcription of RNA that forms double-stranded RNA (dsRNA) having homology to a gene targeted for suppression. Suppression can also be achieved by insertion mutations created by transposable elements can also prevent gene function. For example, in many dicot plants, transformation with the T-DNA of Agrobacterium can be readily achieved and large numbers of transformants can be rapidly obtained. Also, some species have lines with active transposable elements that can efficiently he used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants can be screened with polynucleotides encoding the polypeptide of interest to detect mutated plants having an insertion in the gene encoding the polypeptide of interest.

Gene stacking

The present invention also contemplates that the trait-improving recombinant DNA provided herein can be used in combination with other recombinant DNA to create plants with multiple desired traits or a further enhanced trait. The combinations generated can include multiple copies of any one or more of the recombinant DNA constructs. These stacked combinations can be created by any method, including but not limited to cross breeding of transgenic plants, or multiple genetic transformation.

### Transformation Methods

Numerous methods for producing plant cell nuclei with recombinant DNA are known in the art and can be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S.. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,463,174 (canola), U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

Numerous methods for transforming chromosomes in a plant cell nucleus with recombinant DNA are known in the art and are used in methods of preparing a transgenic plant cell nucleus cell, and plant. Two effective methods for such transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice) and Agrobacterium-mediated transformation is described in U.S. Pat. No. 5,159,135 (cotton); U.S. Pat. No. 5,824,877 (soybean); U.S. Pat. No. 5,463,174 (canola, also known as rapeseed); U.S. Pat. No. 5,591,616 (corn); U.S. Pat. No. 6,384,301 (soybean), U.S. Pat. No. 7,026,528 (wheat) and U.S. Pat. No. 6,329,571 (rice), all of which are incorporated herein by reference for enabling the production of transgenic plants. Transformation of plant material is practiced in tissue culture on a nutrient media, i.e. a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In general it is useful to introduce heterologous DNA randomly, i.e., at a non-specific location, in the genome of a target plant line. In special cases it can be useful to target heterologous DNA insertion in order to achieve site-specific integration, e.g., to replace an existing gene in the genome, to use an existing promoter in the plant genome, or to insert a recombinant polynucleotide at a predetermined site known to be active for gene expression. Several site specific recombination systems exist which are known to function in plants including cre-lox as disclosed in U.S. Pat. No. 4,959,317 and FLP-FRT as disclosed in U.S. Pat. No. 5,527,695, both incorporated herein by reference.

Transformation methods of this invention can be practiced in tissue culture on media and in a controlled environment. “Media” refers to the numerous nutrient mixtures that are used to grow cells in vitro, that is, outside of the intact living organism. Recipient cell targets include, but are not limited to, meristem cells, calli, hypocotyles, immature embryos and gametic cells such as microspores, pollen, sperm and egg cells. It is contemplated that any cell from which a fertile plant can be regenerated is useful as a recipient cell. Callus can be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, e.g., various media and recipient target cells, transformation of immature embryos and subsequent regeneration of fertile transgenic plants are disclosed in U.S. Pat. Nos. 6,194,636 and 6,232,526 and U.S. patent application Ser. No. 09/757,089, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into a first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In practice, DNA is introduced into only a small percentage of target cell nuclei. Marker genes are used to provide an efficient system for identification of those cells with nuclei that are stably transformed by receiving and integrating a recombinant DNA molecule into their genomes. Some marker genes provide selective markers that confer resistance to a selective agent, such as an antibiotic or herbicide. Potentially transformed cells with a nucleus of the invention are exposed to the selective agent. In the population of surviving cells will be those cells where, generally, the resistance-conferring gene has been integrated and expressed at sufficient levels to permit cell survival. Cells can be tested further to confirm stable integration of the exogenous DNA in the nucleus. Useful selective marker genes include those conferring resistance to antibiotics such as kanamycin (nptII), hygromycin B (aph IV), spectinomycin (aadA) and gentamycin (aac3 and aacC4) or resistance to herbicides such as glufosinate (bar or pat), dicamba (DMO) and glyphosate (EPSPS). Examples of such selectable markers are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known. It is also contemplated that combinations of screenable and selectable markers will be useful for identification of transformed cells. See PCT publication WO 99/61129 (herein incorporated by reference) which discloses use of a gene fusion between a selectable marker gene and a screenable marker gene, e.g., an NPTII gene and a GFP gene.

Plant cells that survive exposure to the selective agent, or cells that have been scored positive in a screening assay, can be cultured in regeneration media and allowed to mature into plants. Developing plantlets can be transferred to soil less plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are matured either in a growth chamber or greenhouse. Plants are regenerated from about 6 weeks to 10 months after a transformant is identified, depending on the initial tissue. During regeneration, cells are grown to plants on solid media at about 19 to 28° C. After regenerating plants have reached the stage of shoot and root development, they can be transferred to a greenhouse for further growth and testing. Plants can be pollinated using conventional plant breeding methods known to those of skill in the art and seed produced.

Progeny can be recovered from transformed plants and tested for expression of the exogenous recombinant polynucleotide. Useful assays include, for example, “molecular biological” assays, such as Southern and Northern blotting and PCR; “biochemical” assays, such as detecting the presence of RNA, e.g., double stranded RNA, or a protein product, e.g., by immunological means (ELISAs and Western blots) or by enzymatic function; plant part assays, such as leaf or root assays; and also, by analyzing the phenotype of the whole regenerated plant.

### Discovery of Trait-Improving Recombinant DNA

To identify nuclei with recombinant DNA that confer enhanced traits to plants, Arabidopsis thaliana was transformed with a candidate recombinant DNA construct and screened for an enhanced trait.

Arabidopsis thaliana is used a model for genetics and metabolism in plants. A two-step screening process was employed which included two passes of trait characterization to ensure that the trait modification was dependent on expression of the recombinant DNA, but not due to the chromosomal location of the integration of the transgene. Twelve independent transgenic lines for each recombinant DNA construct were established and assayed for the transgene expression levels. Five transgenic lines with high transgene expression levels were used in the first pass screen to evaluate the transgene's function in T2 transgenic plants. Subsequently, three transgenic events, which had been shown to have one or more enhanced traits, were further evaluated in the second pass screen to confirm the transgene's ability to impart an enhanced trait. The following Table 3 summarizes the enhanced traits that have been confirmed as provided by a recombinant DNA construct.

In particular, Table 3 reports:

“PEP SEQ ID” which is the amino acid sequence of the protein cognate to the DNA in the recombinant DNA construct corresponding to a protein sequence of a SEQ ID NO. in the Sequence Listing.

“construct_id” is an arbitrary name for the recombinant DNA describe more particularly in Table 1.

“annotation” refers to a description of the top hit protein obtained from an amino acid sequence query of each PEP SEQ ID NO to GenBank database of the National Center for Biotechnology Information (ncbi). More particularly, “gi” is the GenBank ID number for the top BLAST hit.

“ description” refers to the description of the top BLAST hit.

“e-value” provides the expectation value for the BLAST hit.

“% id” refers to the percentage of identically matched amino acid residues along the length of the portion of the sequences which is aligned by BLAST between the sequence of interest provided herein and the hit sequence in GenBank.

“traits” identify by two letter codes the confirmed enhancement in a transgenic plant provided by the recombinant DNA . The codes for enhanced traits are:

“CK” which indicates cold tolerance enhancement identified under a cold shock tolerance screen;

“CS” which indicates cold tolerance enhancement identified by a cold germination tolerance screen;

“DS” which indicates drought tolerance enhancement identified by a soil drought stress tolerance screen;

“PEG” which indicates osmotic stress tolerance enhancement identified by a PEG induced osmotic stress tolerance screen;

“HS” which indicates heat stress tolerance enhancement identified by a heat stress tolerance screen;

“SS” which indicates high salinity stress tolerance enhancement identified by a salt stress tolerance screen;

“LN” which indicates nitrogen use efficiency enhancement identified by a limited nitrogen tolerance screen;

“LL” which indicates attenuated shade avoidance response identified by a shade tolerance screen under a low light condition;

“PP” which indicates enhanced growth and development at early stages identified by an early plant growth and development screen;

“SP” which indicates enhanced growth and development at late stages identified by a late plant growth and development screen provided herein.

TABLE 3

| PEP | | | | | | | | | |
|---|---|---|---|---|---|---|---|---|---|
| SEQ | | Annotation | | | | | | | |
| ID | | % | | | | | | | |
| NO | Construct | Id | E-value | Description | traits | | | | |
| 115 | 10177 | 86 | 1.00E−100 | ref\|NP_188965.1\|ATERF1/ERF1; DNA | LN | | | | |
| | | | | binding/transcription factor/transcriptional | | | | | |
| | | | | activator [Arabidopsis thaliana] | | | | | |
| 116 | 12155 | 85 | 1.00E−178 | ref\|NP_565684.1\|nucleic acid binding/ | CS | SS | | | |
| | | | | transcription factor/zinc ion binding | | | | | |
| | | | | [Arabidopsis thaliana] | | | | | |
| 117 | 12233 | 100 | 1.00E−144 | gb\|AAG51950.1\|AC015450_11unknown | LN | SS | | | |
| | | | | protein; 77280-78196 [Arabidopsis thaliana] | | | | | |
| 118 | 11873 | 91 | 0 | ref\|NP_187289.1\|phosphoric diester | LN | PEG | | | |
| | | | | hydrolase/transcription factor | | | | | |
| | | | | [Arabidopsis thaliana] | | | | | |
| 119 | 17308 | 94 | 0 | ref\|NP_198771.1\|protein binding/ | CK | | | | |
| | | | | ubiquitin-protein ligase/zinc ion binding | | | | | |
| | | | | [Arabidopsis thaliana] | | | | | |
| 120 | 78706 | 80 | 2.00E−75 | ref\|NP_18145.1\|SPL5 (SQUAMOSA | PEG | | | | |
| | | | | PROMOTER BINDING PROTEIN-LIKE | | | | | |
| | | | | 5); DNA binding/transcription factor | | | | | |
| | | | | [Arabidopsis thaliana] | | | | | |
| 121 | 72666 | 100 | 1.00E−135 | ref\|NP_181657.1\|DNA binding/ | SP | | | | |
| | | | | transcription factor [Arabidopsis thaliana] | | | | | |
| | | | | gb\|AAC78547.1\| | | | | | |
| 122 | 17520 | 74 | 4.00E−75 | gb\|ABK28523.1\|unknown | CK | HS | PP | PEG | |
| | | | | [Arabidopsis thaliana] | | | | | |
| 123 | 72672 | 100 | 8.00E−91 | ref\|NP_192762.1\|transcription factor/zinc | CS | | | | |
| | | | | ion binding [Arabidopsis thaliana] | | | | | |
| 124 | 78977 | 80 | 2.00E−83 | ref\|NP_173506.1\|protein binding/ | LN | | | | |
| | | | | ubiquitin-protein ligase/zinc ion binding | | | | | |
| | | | | [Arabidopsis thaliana] | | | | | |
| 125 | 17468 | 75 | 1.00E−109 | ref\|NP_195167.1\|DNA binding/ | CS | | | | |
| | | | | transcription factor [Arabidopsis thaliana] | | | | | |
| 126 | 75090 | 70 | 1.00E−166 | ref\|NP_194111.1\|transcription factor | HS | PP | SS | PEG | |
| | | | | [Arabidopsis thaliana] | | | | | |
| 127 | 72018 | 85 | 5.00E−85 | ref\|NP_179337.1\|RHA3A; protein binding/ | DS | LL | LN | | |
| | | | | ubiquitin-protein ligase/zinc ion binding | | | | | |
| | | | | [Arabidopsis thaliana] | | | | | |
| 128 | 17524 | 97 | 0 | ref\|NP_200014.1\|nucleic acid binding/ | CK | LL | PEG | | |
| | | | | transcription factor/zinc ion binding | | | | | |
| | | | | [Arabidopsis thaliana] | | | | | |
| 129 | 72078 | 74 | 1.00E−126 | ref\|NP_190677.1\|transcription factor | LL | | | | |
| | | | | [Arabidopsis thaliana] | | | | | |
| 130 | 72977 | 94 | 0 | ref\|NP_850745.1\|DNA binding/ | CK | LN | | | |
| | | | | transcription factor [Arabidopsis thaliana] | | | | | |
| 131 | 17445 | 72 | 1.00E−100 | ref\|NP_191608.1\|DNA binding/ | LN | | | | |
| | | | | transcription factor [Arabidopsis thaliana] | | | | | |
| 132 | 78979 | 82 | 1.00E−153 | ref\|NP_191212.1\|ANAC064; transcription | LL | LN | | | |
| | | | | factor [Arabidopsis thaliana] | | | | | |
| 133 | 18453 | 69 | 1.00E−154 | ref\|NP_174001.1\|DNA binding/ | CS | HS | PP | SS | |
| | | | | transcription factor [Arabidopsis thaliana] | | | | | |
| 134 | 17913 | 88 | 8.00E−69 | ref\|NP_196812.1\|WRKY75; transcription | CK | HS | | | |
| | | | | factor [Arabidopsis thaliana] | | | | | |
| 135 | 18210 | 78 | 2.00E−82 | ref\|NP_563624.1\|DNA binding/ | PEG | | | | |
| | | | | transcription factor [Arabidopsis thaliana] | | | | | |
| 136 | 78710 | 93 | 1.00E−154 | gb\|AAF79495.1\|AC002328_3F20N2.8 | HS | LN | | | |
| | | | | [Arabidopsis thaliana] | | | | | |
| 137 | 73606 | 83 | 1.00E−108 | ref\|NP_196295.1\|nucleic acid binding/ | CS | HS | SS | | |
| | | | | transcription factor [Arabidopsis thaliana] | | | | | |
| 138 | 18446 | 100 | 1.00E−166 | ref\|NP_176212.1\|transcription factor | SS | | | | |
| | | | | [Arabidopsis thaliana] | | | | | |
| 139 | 75011 | 78 | 1.00E−134 | ref\|NP_195651.1\|WRKY13; transcription | PEG | | | | |
| | | | | factor [Arabidopsis thaliana] | | | | | |
| 140 | 18507 | 68 | 1.00E−119 | ref\|NP_191581.1\|ATL4; protein binding/ | CK | SS | | | |
| | | | | ubiquitin-protein ligase/zinc ion binding | | | | | |
| | | | | [Arabidopsis thaliana] | | | | | |
| 141 | 18388 | 93 | 1.00E−133 | ref\|NP_190147.1\|DNA binding/ | SS | | | | |
| | | | | transcription factor [Arabidopsis thaliana] | | | | | |
| 142 | 18540 | 82 | 1.00E−110 | ref\|NP_193836.1\|DNA binding/ | PP | PEG | | | |
| | | | | transcription factor [Arabidopsis thaliana] | | | | | |
| 143 | 19653 | 83 | 3.00E−72 | gb\|ABB02372.1\|AP2/EREBP transcription | CS | LN | | | |
| | | | | factor [Arabidopsis thaliana] | | | | | |
| 144 | 18411 | 80 | 3.00E−93 | gb\|AAD20668.1\|ethylene reponse factor- | CS | HS | | | |
| | | | | like AP2 domain transcription factor | | | | | |
| | | | | [Arabidopsis thaliana] | | | | | |
| 145 | 70737 | 100 | 0 | ref\|NP_564265.1\|unknown protein | CS | HS | SS | | |
| | | | | [Arabidopsis thaliana] | | | | | |
| 146 | 18315 | 72 | 3.00E−90 | ref\|NP_196054.1\|C2H2; nucleic acid | SS | PEG | | | |
| | | | | binding/transcription factor/zinc ion | | | | | |
| | | | | binding [Arabidopsis thaliana] | | | | | |
| 147 | 75080 | 94 | 1.00E−117 | ref\|NP_179033.1\|ANR1; DNA binding/ | HS | PEG | | | |
| | | | | transcription factor [Arabidopsis thaliana] | | | | | |
| 148 | 19191 | 71 | 6.00E−68 | gb\|AAK76521.1\|putative Dof zinc finger | SS | | | | |
| | | | | protein [Arabidopsis thaliana] | | | | | |
| 149 | 18431 | 90 | 0 | ref\|NP_192961.1\|nucleic acid binding/ | LN | | | | |
| | | | | transcription factor/zinc ion binding | | | | | |
| | | | | [Arabidopsis thaliana] | | | | | |
| 150 | 18433 | 89 | 1.00E−163 | ref\|NP_195443.1\|MYB73; DNA binding/ | SP | | | | |
| | | | | transcription factor [Arabidopsis thaliana] | | | | | |
| 151 | 18434 | 72 | 1.00E−122 | ref\|NP_195758.1\|transcription factor | HS | | | | |
| | | | | [Arabidopsis thaliana] | | | | | |
| 152 | 19536 | 93 | 0 | ref\|NP_568099.1\|MYB3R-5; DNA binding/ | PEG | | | | |
| | | | | transcription factor [Arabidopsis thaliana] | | | | | |
| 153 | 76577 | 93 | 8.00E−84 | ref\|NP_180991.1\|transcription factor | PEG | | | | |
| | | | | [Arabidopsis thaliana] | | | | | |
| 154 | 11113 | 92 | 0 | ref\|NP_565621.1\|PEX10; protein binding/ | CS | | | | |
| | | | | ubiquitin-protein ligase/zinc ion binding | | | | | |
| | | | | [Arabidopsis thaliana] | | | | | |
| 155 | 19614 | 100 | 1.00E−84 | ref\|NP_566925.1\|DNA binding/ | PEG | | | | |
| | | | | transcription factor [Arabidopsis thaliana] | | | | | |
| 156 | 18204 | 85 | 0 | ref\|NP_001031255.1\|unknown protein | CK | | | | |
| | | | | [Arabidopsis thaliana] | | | | | |
| 157 | 73614 | 96 | 0 | ref\|NP_186995.1\|RGL2 (RGA-LIKE 2); | CS | HS | | | |
| | | | | transcription factor [Arabidopsis thaliana] | | | | | |
| 158 | 70450 | 76 | 1.00E−138 | ref\|NP_187687.1\|DNA binding/ | CS | HS | | | |
| | | | | transcription factor [Arabidopsis thaliana] | | | | | |
| 159 | 70618 | 94 | 0 | ref\|NP_188000.1\|transcription factor | HS | SS | | | |
| | | | | [Arabidopsis thaliana] | | | | | |
| 160 | 70458 | 95 | 0 | ref\|NP_172779.1\|unknown protein | LL | | | | |
| | | | | [Arabidopsis thaliana] | | | | | |
| 161 | 71306 | 81 | 0 | ref\|NP_195480.1\|SHR (SHORT ROOT); | CS | HS | SP | PEG | |
| | | | | transcription factor [Arabidopsis thaliana] | | | | | |
| 162 | 70451 | 92 | 1.00E−141 | ref\|NP_195810.2\|WRKY62; transcription | SS | | | | |
| | | | | factor [Arabidopsis thaliana] | | | | | |
| 163 | 70453 | 71 | 1.00E−154 | ref\|NP_201032.2\|DNA binding | CS | | | | |
| | | | | [Arabidopsis thaliana] | | | | | |
| 164 | 71311 | 89 | 0 | ref\|NP_176498.1\|transcription factor | CS | LN | | | |
| | | | | [Arabidopsis thaliana] | | | | | |
| 165 | 76533 | 91 | 0 | gb\|ABK28765.1\|unknown | HS | | | | |
| | | | | [Arabidopsis thaliana] | | | | | |
| 166 | 70955 | 87 | 1.00E−178 | gb\|AAX21351.1\|phantastica transcription | SP | | | | |
| | | | | factor a [Glycine max] | | | | | |
| 167 | 77708 | 57 | 1.00E−64 | ref\|NP_200279.1\|DNA binding/ | DS | | | | |
| | | | | transcription factor [Arabidopsis thaliana] | | | | | |
| 168 | 70954 | 61 | 9.00E−72 | emb\|CAB77055.1\|putative TFIIIA (or | LN | PEG | | | |
| | | | | kruppel)-like zinc finger protein | | | | | |
| | | | | [Medicago sativa subsp. × varia] | | | | | |
| 169 | 19742 | 86 | 1.00E−156 | gb\|AAK84883.1\|AF402602_1NAC domain | CS | | | | |
| | | | | protein NAC1 [Phaseolus vulgaris] | | | | | |
| 170 | 70960 | 53 | 1.00E−72 | ref\|NP_565183.1\|transcription factor/zinc | PEG | | | | |
| | | | | ion binding [Arabidopsis thaliana] | | | | | |
| 171 | 19940 | 94 | 0 | gb\|ABH02845.1\|MYB transcription factor | PEG | | | | |
| | | | | MYB93 [Glycine max] | | | | | |
| 172 | 70921 | 88 | 1.00E−156 | gb\|AAX85979.1\|NAC2 protein [Glycine | CS | CK | | | |
| | | | | max] | | | | | |
| 173 | 70953 | 83 | 1.00E−121 | gb\|ABI97244.1\|PHD5 [Glycine max] | DS | PP | SS | PEG | |
| 174 | 70978 | 99 | 1.00E−134 | emb\|CAI47596.1\|MADS transcription factor | HS | PEG | | | |
| | | | | [Glycine max] | | | | | |
| 175 | 70940 | 47 | 1.00E−97 | ref\|NP_191362.2\|protein binding/ | SS | | | | |
| | | | | ubiquitin-protein ligase/zinc ion binding | | | | | |
| | | | | [Arabidopsis thaliana] | | | | | |
| 176 | 70977 | 57 | 8.00E−76 | ref\|NP_567245.1\|DNA binding/ | HS | | | | |
| | | | | transcription factor [Arabidopsis thaliana] | | | | | |
| 177 | 70993 | 92 | 1.00E−105 | gb\|AAX13302.1\|MADS box protein AP3- | CS | SS | | | |
| | | | | like [Lotus corniculatus var. japonicus] | | | | | |
| 178 | 19762 | 98 | 2.00E−36 | gb\|ABH02867.1\|MYB transcription factor | HS | PP | | | |
| | | | | MYB142 [Glycine max] | | | | | |
| 179 | 70938 | 47 | 2.00E−63 | dbj\|BAB16432.1\|WRKY transcription | CS | HS | PP | PEG | |
| | | | | factor NtEIG-D48 [Nicotiana tabacum] | | | | | |
| 180 | 78653 | 100 | 1.00E−114 | ref\|NP_199999.1\|transcription factor | LL | LN | | | |
| | | | | [Arabidopsis thaliana] | | | | | |
| 181 | 73216 | 99 | 0 | ref\|NP_189228.1\|transcription factor | LL | | | | |
| | | | | [Arabidopsis thaliana] | | | | | |
| 182 | 71139 | 91 | 1.00E−152 | ref\|NP_187669.1\|DNA binding/ | LN | | | | |
| | | | | transcription factor [Arabidopsis thaliana] | | | | | |
| 183 | 74227 | 74 | 1.00E−174 | ref\|NP_187615.2\|transcription factor | LL | | | | |
| | | | | [Arabidopsis thaliana] | | | | | |
| 184 | 70678 | 91 | 1.00E−142 | ref\|NP_173195.2\|DNA binding/ | LN | LL | | | |
| | | | | transcription factor [Arabidopsis thaliana] | | | | | |
| 185 | 70769 | 90 | 1.00E−148 | ref\|NP_194286.1\|DNA binding/ | LL | | | | |
| | | | | transcription factor [Arabidopsis thaliana] | | | | | |
| 186 | 71643 | 83 | 1.00E−141 | gb\|ABH02851.1\|MYB transcription factor | LL | LN | | | |
| | | | | MYB109 [Glycine max] | | | | | |
| 187 | 70242 | 93 | 0 | ref\|NP_565162.1\|calmodulin binding/ | DS | | | | |
| | | | | transcription factor [Arabidopsis thaliana] | | | | | |
| 188 | 12325 | 94 | 1.00E−164 | ref\|NP_191684.1\|DNA binding/ | LN | | | | |
| | | | | transcription factor [Arabidopsis thaliana] | | | | | |
| 189 | 70356 | 95 | 1.00E−114 | gb\|AAX13306.1\|MADS box protein AGL11 | DS | | | | |
| | | | | [Lotus corniculatus var. japonicus] | | | | | |
| 190 | 72034 | 72 | 3.00E−90 | gb\|AAX69070.1\|MADS box protein M8 | LL | | | | |
| | | | | [Pisum sativum] | | | | | |
| 191 | 72046 | 57 | 1.00E−64 | dbj\|BAD15085.1\|CCAAT-box binding | CS | PP | SS | | |
| | | | | factor HAP5 homolog [Daucus carota] | | | | | |
| 192 | 72071 | 76 | 1.00E−79 | sp\|P32294\|AX22B_PHAAUAuxin-induced | PEG | | | | |
| | | | | protein 22B (Indole-3-acetic acid-induced | | | | | |
| | | | | protein ARG4) dbj\|BAA03309.1\|ORF | | | | | |
| | | | | [Vigna radiata] | | | | | |
| 193 | 72116 | 93 | 1.00E−123 | dbj\|BAB86892.1\|syringolide-induced | HS | LN | SS | | |
| | | | | protein 1-3-1A [Glycine max] | | | | | |
| 194 | 72108 | 82 | 1.00E−87 | gb\|ABK58464.1\|LIM domain protein | LL | | | | |
| | | | | GLIM1a [Populus tremula × Populus alba] | | | | | |
| 195 | 77605 | 99 | 0 | gb\|ABE65879.1\|zinc finger family protein | CK | HS | LL | PEG | PP |
| | | | | [Arabidopsis thaliana] | | | | | |
| 196 | 73978 | 90 | 0 | ref\|NP_179919.1\|CLF (CURLY LEAF); | SS | PEG | | | |
| | | | | transcription factor [Arabidopsis thaliana] | | | | | |
| 197 | 73032 | 69 | 1.00E−115 | gb\|ABI34669.1\|bZIP transcription factor | LL | | | | |
| | | | | bZIP131 [Glycine max] | | | | | |
| 198 | 11157 | 96 | 0 | ref\|NP_177279.1\|AJH2 | LN | | | | |
| | | | | [Arabidopsis thaliana] | | | | | |
| 199 | 73493 | 97 | 0 | ref\|NP_010736.1\|Ada2p | PEG | | | | |
| | | | | [Saccharomyces cerevisiae] | | | | | |
| 200 | 71228 | 93 | 1.00E−135 | ref\|NP_565948.1\|EEL (ENHANCED EM | CK | | | | |
| | | | | LEVEL); DNA binding/transcription | | | | | |
| | | | | factor [Arabidopsis thaliana] | | | | | |
| 201 | 74889 | 64 | 1.00E−118 | gb\|ABE82375.1\|Transcription factor | CS | | | | |
| | | | | E2F/dimerisation partner (TDP) | | | | | |
| | | | | [Medicago truncatula] | | | | | |
| 202 | 74854 | 75 | 1.00E−115 | gb\|ABH02826.1\|MYB transcription factor | LN | | | | |
| | | | | MYB55 [Glycine max] | | | | | |
| 203 | 74891 | 88 | 1.00E−67 | gb\|AAL31068.1\|AC090120_14putative c- | CS | DS | LN | | |
| | | | | myc binding protein [Oryza sativa] | | | | | |
| 204 | 75314 | 51 | 5.00E−71 | ref\|NP_181828.1\|ANAC042; transcription | DS | LN | | | |
| | | | | factor [Arabidopsis thaliana] | | | | | |
| 205 | 75340 | 66 | 3.00E−88 | emb\|CAA63222.1\|homeobox-leucine zipper | LL | | | | |
| | | | | protein [Glycine max] | | | | | |
| 206 | 75329 | 91 | 1.00E−62 | ref\|NP_001062531.1\|Os08g0564500 | PEG | | | | |
| | | | | [Oryza sativa (japonica cultivar-group)] | | | | | |
| 207 | 75453 | 81 | 1.00E−140 | gb\|ABH02828.1\|MYB transcription factor | HS | LN | | | |
| | | | | MYB57 [Glycine max] | | | | | |
| 208 | 75477 | 48 | 5.00E−38 | gb\|ABC69353.1\|ethylene-responsive | LL | PEG | | | |
| | | | | element-binding protein | | | | | |
| | | | | [Medicago truncatula] | | | | | |
| 209 | 75430 | 69 | 9.00E−30 | emb\|CAC28528.1\|GATA-1 zinc finger | LL | LN | PEG | | |
| | | | | protein [Nicotiana tabacum] | | | | | |
| 210 | 75467 | 43 | 3.00E−30 | gb\|AAM61659.1\|unknown | CS | HS | | | |
| | | | | [Arabidopsis thaliana] | | | | | |
| 211 | 75457 | 72 | 1.00E−89 | gb\|AAX69065.1\|MADS box protein M2 | PP | SP | | | |
| | | | | [Pisum sativum] | | | | | |
| 212 | 75469 | 91 | 1.00E−143 | gb\|ABI34650.1\|bZIP transcription factor | SP | | | | |
| | | | | bZIP68 [Glycine max] | | | | | |
| 213 | 75470 | 74 | 2.00E−44 | gb\|AAP13348.1\|transcription factor GT-3b | HS | PP | | | |
| | | | | [Arabidopsis thaliana] | | | | | |
| 214 | 75435 | 71 | 1.00E−28 | ref\|NP_566164.1\|PTF1 (PLASTID | DS | | | | |
| | | | | TRANSCRIPTION FACTOR 1); | | | | | |
| | | | | transcription factor [Arabidopsis thaliana] | | | | | |
| 215 | 77822 | 61 | 2.00E−75 | gb\|AAN05420.1\|putative RING protein | DS | LL | | | |
| | | | | [Populus × canescens] | | | | | |
| 216 | 75505 | 49 | 1.00E−172 | gb\|ABI30334.1\|Cys-3-His zinc finger | PP | | | | |
| | | | | protein [Capsicum annuum] | | | | | |
| 217 | 75589 | 56 | 1.00E−115 | gb\|ABE81808.1\|Homeodomain-related | LN | | | | |
| | | | | [Medicago truncatula] | | | | | |
| 218 | 75542 | 69 | 9.00E−83 | gb\|ABE78575.1\|TCP transcription factor | LN | SP | | | |
| | | | | [Medicago truncatula] | | | | | |
| 219 | 75545 | 42 | 2.00E−36 | gb\|AAQ56115.1\|transcription-factor-like | SS | | | | |
| | | | | protein [Boechera drummondii] | | | | | |
| 220 | 75581 | 44 | 1.00E−89 | ref\|NP_189124.1\|PRT1 (PROTEOLYSIS | LL | | | | |
| | | | | 1); ubiquitin-protein ligase | | | | | |
| | | | | [Arabidopsis thaliana] | | | | | |
| 221 | 75524 | 50 | 1.00E−106 | ref\|NP_176980.1\|nucleic acid binding/ | LN | | | | |
| | | | | transcription factor/zinc ion binding | | | | | |
| | | | | [Arabidopsis thaliana] | | | | | |
| 222 | 75658 | 49 | 6.00E−29 | gb\|AAV85852.1\|AT-rich element binding | LL | | | | |
| | | | | factor 2 [Pisum sativum] | | | | | |
| 223 | 75738 | 67 | 6.00E−68 | gb\|AAM33103.2\|TAGL12 transcription | LL | SP | | | |
| | | | | factor [Lycopersicon esculentum] | | | | | |
| 224 | 77356 | 94 | 1.00E−119 | ref\|NP_564547.1\|RTV1; DNA binding/ | LL | | | | |
| | | | | transcription factor [Arabidopsis thaliana] | | | | | |
| 225 | 77362 | 100 | 3.00E−73 | ref\|NP_193892.1\|LOL2 (LSD ONE LIKE | DS | | | | |
| | | | | 2); transcription factor | | | | | |
| | | | | [Arabidopsis thaliana] | | | | | |
| 226 | 77363 | 67 | 1.00E−47 | ref\|NP_194747.1\|transcription factor/ | LN | | | | |
| | | | | transcription regulator | | | | | |
| | | | | [Arabidopsis thaliana] | | | | | |
| 227 | 77371 | 95 | 1.00E−127 | ref\|NP_199178.1\|DNA binding/ | CS | | | | |
| | | | | transcription factor [Arabidopsis thaliana] | | | | | |
| 228 | 77941 | 90 | 1.00E−140 | dbj\|BAB09311.1\|tumor-related protein-like | DS | SS | | | |
| | | | | [Arabidopsis thaliana] | | | | | |

### Trait Enhancement Screens

DS-Enhancement of Drought Tolerance Identified by a Soil Drought Stress Tolerance Screen: Drought or water deficit conditions impose mainly osmotic stress on plants. Plants are particularly vulnerable to drought during the flowering stage. The drought condition in the screening process disclosed in Example 1B started from the flowering time and was sustained to the end of harvesting. The present invention provides recombinant DNA that can improve the plant survival rate under such sustained drought condition. Exemplary recombinant DNA for conferring such drought tolerance are identified as such in Table 3. Such recombinant DNA can be used in generating transgenic plants that are tolerant to the drought condition imposed during flowering time and in other stages of the plant life cycle. As demonstrated from the model plant screen, in some embodiments of transgenic plants with trait-improving recombinant DNA grown under such sustained drought condition can also have increased total seed weight per plant in addition to the increased survival rate within a transgenic population, providing a higher yield potential as compared to control plants.

PEG-Enhancement of drought tolerance identified by PEG induced osmotic stress tolerance screen: Various drought levels can be artificially induced by using various concentrations of polyethylene glycol (PEG) to produce different osmotic potentials (Pilon-Smits e.g., (1995) Plant Physiol. 107:125-130). Several physiological characteristics have been reported as being reliable indications for selection of plants possessing drought tolerance. These characteristics include the rate of seed germination and seedling growth. The traits can be assayed relatively easily by measuring the growth rate of seedling in PEG solution. Thus, a PEG-induced osmotic stress tolerance screen is a useful surrogate for drought tolerance screen. As demonstrated from the model plant screen, embodiments of transgenic plants with trait-improving recombinant DNA identified in the PEG-induced osmotic stress tolerance screen can survive better drought conditions providing a higher yield potential as compared to control plants.

SS-Enhancement of drought tolerance identified by high salinity stress tolerance screen: Three different factors are responsible for salt damages: (1) osmotic effects, (2) disturbances in the mineralization process, and (3) toxic effects caused by the salt ions, e.g., inactivation of enzymes. While the first factor of salt stress results in the wilting of the plants that is similar to drought effect, the ionic aspect of salt stress is clearly distinct from drought. The present invention provides genes that help plants maintain biomass, root growth, and/or plant development in high salinity conditions, which are identified as such in Table 3. Since osmotic effect is one of the major components of salt stress, which is common to the drought stress, trait-improving recombinant DNA identified in a high salinity stress tolerance screen can also provide transgenic crops with enhanced drought tolerance. As demonstrated from the model plant screen, embodiments of transgenic plants with trait-improving recombinant DNA identified in a high salinity stress tolerance screen can survive better drought conditions and/or high salinity conditions providing a higher yield potential as compared to control plants.

HS-Enhancement of drought tolerance identified by heat stress tolerance screen: Heat and drought stress often occur simultaneously, limiting plant growth. Heat stress can cause the reduction in photosynthesis rate, inhibition of leaf growth and osmotic potential in plants. Thus, genes identified by the present invention as heat stress tolerance conferring genes can also impart enhanced drought tolerance to plants. As demonstrated from the model plant screen, embodiments of transgenic plants with trait-improving recombinant DNA identified in a heat stress tolerance screen can survive better heat stress conditions and/or drought conditions providing a higher yield potential as compared to control plants.

CK and CS-Enhancement of tolerance to cold stress: Low temperature can immediately result in mechanical constraints, changes in activities of macromolecules, and reduced osmotic potential. In the present invention, two screening conditions, i.e., cold shock tolerance screen (CK) and cold germination tolerance screen (CS), were set up to look for transgenic plants that display visual growth advantage at lower temperature. In cold germination tolerance screen, the transgenic Arabidopsis plants were exposed to a constant temperature of 8° C. from planting until day 28 post plating. The trait-improving recombinant DNA identified by such screen are particular useful for the production of transgenic plant that can germinate more robustly in a cold temperature as compared to the wild type plants. In cold shock tolerance screen, the transgenic plants were first grown under the normal growth temperature of 22° C. until day 8 post plating, and subsequently were placed under 8° C. until day 28 post plating. As demonstrated from the model plant screen, embodiments of transgenic plants with trait-improving recombinant DNA identified in a cold shock stress tolerance screen and/or a cold germination stress tolerance screen can survive better cold conditions providing a higher yield potential as compared to control plants.

Enhancement of tolerance to multiple stresses: Different kinds of stresses often lead to identical or similar reaction in the plants. Genes that are activated or inactivated as a reaction to stress can either act directly in a way the genetic product reduces a specific stress, or they can act indirectly by activating other specific stress genes. By manipulating the activity of such regulatory genes, i.e., multiple stress tolerance genes, the plant can be enabled to react to different kinds of stresses. For examples, PEP SEQ ID NO: 116 can be used to enhance both salt stress tolerance and cold stress tolerance in plants. Of particular interest, plants transformed with PEP SEQ ID NO: 133 can resist salt stress and cold stress. Plants transformed with PEP SEQ ID NO: 133 can also improve growth in early stage and under osmotic stress. In addition to these multiple stress tolerance genes, the stress tolerance conferring genes provided by the present invention can be used in combinations to generate transgenic plants that can resist multiple stress conditions.

PP-Enhancement of early plant growth and development: It has been known in the art that to minimize the impact of disease on crop profitability, it is important to start the season with healthy and vigorous plants. This means avoiding seed and seedling diseases, leading to increased nutrient uptake and increased yield potential. Traditionally early planting and applying fertilizer are the methods used for promoting early seedling vigor. In early development stage, plant embryos establish only the basic root-shoot axis, a cotyledon storage organ(s), and stem cell populations, called the root and shoot apical meristems that continuously generate new organs throughout post-embryonic development. “Early growth and development” used herein encompasses the stages of seed imbibition through the early vegetative phase. The present invention provides genes that are useful to produce transgenic plants that have advantages in one or more processes including, but not limited to, germination, seedling vigor, root growth and root morphology under non-stressed conditions. The transgenic plants starting from a more robust seedling are less susceptible to the fungal and bacterial pathogens that attach germinating seeds and seedling. Furthermore, seedlings with advantage in root growth are more resistant to drought stress due to extensive and deeper root architecture. Therefore, it can be recognized by those skilled in the art that genes conferring the growth advantage in early stages to plants can also be used to generate transgenic plants that are more resistant to various stress conditions due to enhanced early plant development. The present invention provides such exemplary recombinant DNA that confer both the stress tolerance and growth advantages to plants, identified as such in Table 3, e.g., PEP SEQ ID NO: 126 which can improve the plant early growth and development, and impart salt tolerance to plants. As demonstrated from the model plant screen, embodiments of transgenic plants with trait-improving recombinant DNA identified in the early plant development screen can grow better under non-stress conditions and/or stress conditions providing a higher yield potential as compared to control plants.

SP-Enhancement of late plant growth and development: “Late growth and development” used herein encompasses the stages of leaf development, flower production, and seed maturity. In certain embodiments, transgenic plants produced using genes that confer growth advantages to plants provided by the present invention, identified as such in Table 3, exhibit at least one phenotypic characteristics including, but not limited to, increased rosette radius, increased rosette dry weight, seed dry weight, silique dry weight, and silique length. On one hand, the rosette radius and rosette dry weight are used as the indexes of photosynthesis capacity, and thereby plant source strength and yield potential of a plant. On the other hand, the seed dry weight, silique dry weight and silique length are used as the indexes for plant sink strength, which are considered as the direct determinants of yield. As demonstrated from the model plant screen, embodiments of transgenic plants with trait-improving recombinant DNA identified in the late development screen can grow better and/or have enhanced development during leaf development and seed maturation providing a higher yield potential as compared to control plants.

LL-Enhancement of tolerance to shade stress identified in a low light screen: The effects of light on plant development are especially prominent at the seedling stage. Under normal light conditions with unobstructed direct light, a plant seeding develops according to a characteristic photomorphogenic pattern, in which plants have open and expanded cotyledons and short hypocotyls. Then the plant's energy is devoted to cotyledon and leaf development while longitudinal extension growth is minimized. Under low light condition where light quality and intensity are reduced by shading, obstruction or high population density, a seedling displays a shade-avoidance pattern, in which the seedling displays a reduced cotyledon expansion, and hypocotyls extension is greatly increased. As the result, a plant under low light condition increases significantly its stem length at the expanse of leaf, seed or fruit and storage organ development, thereby adversely affecting of yield. The present invention provides recombinant DNA that enable plants to have an attenuated shade avoidance response so that the source of plant can be contributed to reproductive growth efficiently, resulting higher yield as compared to the wild type plants. As demonstrated from the model plant screen, embodiments of transgenic plants with trait-improving recombinant DNA identified in a shade stress tolerance screen can have attenuated shade response under shade conditions providing a higher yield potential as compared to control plants. The transgenic plants generated by the present invention can be suitable for a higher density planting, thereby resulting increased yield per unit area.

### LN-Enhancement of Tolerance to Low Nitrogen Availability Stress

Nitrogen is a key factor in plant growth and crop yield. The metabolism, growth and development of plants are profoundly affected by their nitrogen supply. Restricted nitrogen supply alters shoot to root ratio, root development, activity of enzymes of primary metabolism and the rate of senescence (death) of older leaves. All field crops have a fundamental dependence on inorganic nitrogenous fertilizer. Since fertilizer is rapidly depleted from most soil types, it must be supplied to growing crops two or three times during the growing season. Enhanced nitrogen use efficiency by plants should enable crops cultivated under low nitrogen availability stress condition resulted from low fertilizer input or poor soil quality.

This invention demonstrates that the transgenic plants generated using the recombinant nucleotides, which confer enhanced nitrogen use efficiency, identified as such in Table 3, exhibit one or more desirable traits including, but not limited to, increased seedling weight, greener leaves, increased number of rosette leaves, increased or decreased root length. One skilled in the art can recognize that the transgenic plants provided by the present invention with enhanced nitrogen use efficiency can also have altered amino acid or protein compositions, increased yield and/or better seed quality. The transgenic plants of the present invention can be productively cultivated under low nitrogcn growth conditions, i.e., nitrogen-poor soils and low nitrogen fertilizer inputs, which would cause the growth of wild type plants to cease or to be so diminished as to make the wild type plants practically useless. The transgenic plants also can be advantageously used to achieve earlier maturing, faster growing, and/or higher yielding crops and/or produce more nutritious foods and animal feedstocks when cultivated using nitrogen non-limiting growth conditions.

Stacked Traits: The present invention also encompasses transgenic plants with stacked engineered traits, e.g., a crop having an enhanced phenotype resulting from expression of a trait-improving recombinant DNA, in combination with herbicide and/or pest resistance traits. For example, genes of the current invention can be stacked with other traits of agronomic interest, such as a trait providing herbicide resistance, for example a RoundUp Ready® trait, or insect resistance, such as using a gene from Bacillus thuringensis to provide resistance against lepidopteran, coliopteran, homopteran, hemioptcran, and other insects. Herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and gluphosinate herbicides. To illustrate that the production of transgenic plants with herbicide resistance is a capability of those of ordinary skill in the art, reference is made to U.S. patent application publications 2003/0106096A1 and 2002/0112260A1 and U.S. Pat. Nos. 5,034,322; 5,776,760, 6,107,549 and 6,376,754, all of which are incorporated herein by reference. To illustrate that the production of transgenic plants with pest resistance is a capability of those of ordinary skill in the art, reference is made to U.S. Pat. Nos. 5,250,515 and 5,880,275 which disclose plants expressing an endotoxin of Bacillus thuringiensis bacteria, to U.S. Pat. No. 6,506,599 which discloses control of invertebrates which feed on transgenic plants which express dsRNA for suppressing a target gene in the invertebrate, to U.S. Pat. No. 5,986,175 which discloses the control of viral pests by transgenic plants which express viral replicase, and to U.S. Patent Application Publication 2003/0150017 A1 which discloses control of pests by a transgenic plant which express a dsRNA targeted to suppressing a gene in the pest, all of which are incorporated herein by reference.

Once one recombinant DNA has been identified as conferring an enhanced trait of interest in transgenic Arabidopsis plants, several methods are available for using the sequence of that recombinant DNA and knowledge about the protein it encodes to identify homologs of that sequence from the same plant or different plant species or other organisms, e.g., bacteria and yeast. Thus, in one aspect, the invention provides methods for identifying a homologous gene with a DNA sequence homologous to any of SEQ ID NO: 1 through SEQ ID NO: 114, or a homologous protein with an amino acid sequence homologous to any of SEQ ID NO: 115 through SEQ ID NO: 228. In another aspect, the present invention provides the protein sequences of identified homologs for a sequence listed as SEQ ID NO: 229 through SEQ ID NO: 4815. In yet another aspect, the present invention also includes linking or associating one or more desired traits, or gene function with a homolog sequence provided herein.

The trait-improving recombinant DNA and methods of using such trait-improving recombinant DNA for generating transgenic plants with enhanced traits provided by the present invention are not limited to any particular plant species. Indeed, the plants according to the present invention can be of any plant species, i.e., can be monocotyledonous or dicotyledonous. In one embodiment, they will be agricultural useful plants, i.e., plants cultivated by man for purposes of food production or technical, particularly industrial applications. Of particular interest in the present invention are corn and soybean plants. The recombinant DNA constructs optimized for soybean transformation and recombinant DNA constructs optimized for corn transformation are provided by the present invention. Other plants of interest in the present invention for production of transgenic plants having enhanced traits include, without limitation, cotton, canola, wheat, sunflower, sorghum, alfalfa, barley, millet, rice, tobacco, fruit and vegetable crops, and turfgrass.

In certain embodiments, the present invention contemplates to use an orthologous gene in generating the transgenic plants with similarly enhanced traits as the transgenic Arabidopsis counterpart. Enhanced physiological properties in transgenic plants of the present invention can be confirmed in responses to stress conditions, for example in assays using imposed stress conditions to detect enhanced responses to drought stress, nitrogen deficiency, cold growing conditions, or alternatively, under naturally present stress conditions, for example under field conditions. Biomass measures can be made on greenhouse or field grown plants and can include such measurements as plant height, stem diameter, root and shoot dry weights, and, for corn plants, ear length and diameter.

Trait data on morphological changes can be collected by visual observation during the process of plant regeneration as well as in regenerated plants transferred to soil. Such trait data includes characteristics such as normal plants, bushy plants, taller plants, thicker stalks, narrow leaves, striped leaves, knotted phenotype, chlorosis, albino, anthocyanin production, or altered tassels, ears or roots. Other enhanced traits can be identified by measurements taken under field conditions, such as days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, trait characteristics of harvested grain can be confirmed, including number of kernels per row on the ear, number of rows of kernels on the ear, kernel abortion, kernel weight, kernel size, kernel density and physical grain quality.

To confirm hybrid yield in transgenic corn plants expressing genes of the present invention, it can be desirable to test hybrids over multiple years at multiple locations in a geographical location where maize is conventionally grown, e.g., in Iowa, Illinois or other locations in the midwestern United States, under “normal” field conditions as well as under stress conditions, e.g., under drought or population density stress.

Transgenic plants can be used to provide plant parts according to the invention for regeneration or tissue culture of cells or tissues containing the constructs described herein. Plant parts for these purposes can include leaves, stems, roots, flowers, tissues, epicotyl, meristems, hypocotyls, cotyledons, pollen, ovaries, cells and protoplasts, or any other portion of the plant which can be used to regenerate additional transgenic plants, cells, protoplasts or tissue culture. Seeds of transgenic plants are provided by this invention can be used to propagate more plants containing the trait-improving recombinant DNA constructs of this invention. These descendants are intended to be included in the scope of this invention if they contain a trait-improving recombinant DNA construct of this invention, whether or not these plants are selfed or crossed with different varieties of plants. The various aspects of the invention are illustrated by means of the following examples which are in no way intended to limit the full breath and scope of claims.

### EXAMPLES

### Example 1

### Identification of Recombinant DNA That Confers Enhanced Trait(s) to Plants

### A. Plant Expression Constructs for Arabidopsis Transformation

Each gene of interest was amplified from a genomic or cDNA library using primers specific to sequences upstream and downstream of the coding region. Transformation vectors were prepared to constitutively transcribe DNA in either sense orientation (for enhanced protein expression) or anti-sense orientation (for endogenous gene suppression) under the control of an enhanced Cauliflower Mosaic Virus 35S promoter (U.S. Pat. No. 5,359,142) directly or indirectly (Moore, e.g., PNAS 95:376-381, 1998; Guyer, e.g., Genetics 149: 633-639, 1998; International patent application NO. PCT/EP98107577). The transformation vectors also contain a bar gene as a selectable marker for resistance to glufosinate herbicide. The transformation of Arabidopsis plants was carried out using the vacuum infiltration method known in the art (Bechtold, e.g., Methods Mol. Biol. 82:259-66, 1998). Seeds harvested from the plants, named as T1 seeds, were subsequently grown in a glufosinate-containing selective medium to select for plants which were actually transformed and which produced T2 transgenic seed.

### B. Soil Drought Tolerance Screen

This example describes a soil drought tolerance screen to identify Arabidopsis plants transformed with recombinant DNA that wilt less rapidly and/or produce higher seed yield when grown in soil under drought conditions

T2 seeds were sown in flats filled with Metro/Mix® 200 (The Scotts® Company, USA). Humidity domes were added to each flat and flats were assigned locations and placed in climate-controlled growth chambers. Plants were grown under a temperature regime of 22° C. at day and 20° C. at night, with a photoperiod of 16 hours and average light intensity of 170 μmol/m²/s. After the first true leaves appeared, humidity domes were removed. The plants were sprayed with glufosinate herbicide and put back in the growth chamber for 3 additional days. Flats were watered for 1 hour the week following the herbicide treatment. Watering was continued every seven days until the flower bud primordia became apparent, at which time plants were watered for the last time.

To identify drought tolerant plants, plants were evaluated for wilting response and seed yield. Beginning ten days after the last watering, plants were examined daily until 4 plants/line had wilted. In the next six days, plants were monitored for wilting response. Five drought scores were assigned according to the visual inspection of the phenotypes: 1 for healthy, 2 for dark green, 3 for wilting, 4 severe wilting, and 5 for dead. A score of 3 or higher was considered as wilted.

At the end of this assay, seed yield measured as seed weight per plant under the drought condition was characterized for the transgenic plants and their controls and analyzed as a quantitative response according to example 1M.

Two approaches were used for statistical analysis on the wilting response. First, the risk score was analyzed for wilting phenotype and treated as a qualitative response according to the example 1L. Alternatively, the survival analysis was carried out in which the proportions of wilted and non-wilted transgenic and control plants were compared over each of the six days under scoring and an overall log rank test was performed to compare the two survival curves using S-PLUS statistical software (S-PLUS 6, Guide to statistics, Insightful, Seattle, Wash., USA). A list of recombinant DNA constructs which improve drought tolerance in transgenic plants is illustrated in Table 4

TABLE 4

| | | | | | | | Time to | |
|---|---|---|---|---|---|---|---|---|
| | | | | | | | wilting | |
| PEP | | | Drought score | | Seed yield | | Risk | |
| SEQ | Construct | | Delta | | Delta | | score | |
| ID NO | ID | Orientation | mean | P-value | mean | P-value | mean | P-value |
| 187 | 70242 | SENSE | 0.034 | 0.539 | 0.783 | 0.029 | −0.047 | 1.000 |
| 189 | 70356 | SENSE | −0.170 | 0.013 | 0.193 | 0.053 | −0.170 | 1.000 |
| 176 | 70977 | SENSE | 0.010 | 0.815 | 0.652 | 0.015 | 0.115 | 1.000 |
| 214 | 75435 | SENSE | 0.312 | 0.021 | −2.969 | 0.004 | 0.248 | 1.000 |
| 225 | 77362 | SENSE | 0.076 | 0.310 | 0.420 | 0.068 | 0.060 | 0.920 |
| 167 | 77708 | SENSE | 0.172 | 0.072 | 0.342 | 0.017 | −0.038 | 0.064 |
| 129 | 72078 | SENSE | 1.794 | 0.006 | / | / | / | / |

Transgenic plants including recombinant DNA expressing protein as set forth in SEQ ID NO: 127, 173, 203, 204, 215, or 228 showed enhanced drought tolerance by the second criterial as illustrated in Example 1L.

### C. Heat Stress Tolerance Screen

Under high temperatures, Arabidopsis seedlings become chlorotic and root growth is inhibited. This example sets forth the heat stress tolerance screen to identify Arabidopsis plants transformed with the gene of interest that are more resistant to heat stress based on primarily their seedling weight and root growth under high temperature.

T2 seeds were plated on ½× MS salts, 1/% phytagel, with 10 μg/ml BASTA (7 per plate with 2 control seeds; 9 seeds total per plate). Plates were placed at 4° C. for 3 days to stratify seeds. Plates were then incubated at room temperature for 3 hours and then held vertically for 11 additional days at temperature of 34° C. at day and 20° C. at night. Photoperiod was 16 h. Average light intensity was ˜140 μmol/m²/s. After 14 days of growth, plants were scored for glufosinate resistance, root length, final growth stage, visual color, and seedling fresh weight. A photograph of the whole plate was taken on day 14.

The seedling weight and root length were analyzed as quantitative responses according to example 1M. The final grow stage at day 14 was scored as success if 50% of the plants had reached 3 rosette leaves and size of leaves are greater than 1 mm (Boyes, e.g., (2001) The Plant Cell 13, 1499-1510). The growth stage data was analyzed as a qualitative response according to example 1L. A list of recombinant DNA constructs that improve heat tolerance in transgenic plants illustrated in Table 5.

TABLE 5

| PEP | | | Root length | | Growth stage | | Seedling weight | |
|---|---|---|---|---|---|---|---|---|
| SEQ ID | Construct | | at day 14 | | at day 14 | | at day 14 | |
| NO | ID | Orientation | Delta | P-value | Risk | P-value | Delta | P-value |
| 126 | 75090 | SENSE | 0.094 | 0.410 | 0.839 | 0.296 | 1.000 | 0.039 |
| 133 | 18453 | SENSE | 0.253 | 0.045 | 0.233 | 0.359 | 0.884 | 0.009 |
| 136 | 78710 | SENSE | 0.073 | 0.574 | 0.139 | 0.681 | 0.606 | 0.008 |
| 137 | 73606 | SENSE | 0.382 | 0.058 | 1.064 | 0.210 | 1.237 | 0.014 |
| 145 | 70737 | SENSE | −0.007 | 0.935 | 0.185 | 0.048 | 0.838 | 0.019 |
| 147 | 75080 | SENSE | 0.306 | 0.171 | 1.172 | 0.242 | 1.140 | 0.021 |
| 151 | 18434 | SENSE | 0.095 | 0.385 | −0.029 | / | / | 0.030 |
| 157 | 73614 | SENSE | −0.051 | 0.238 | −0.178 | 0.377 | 0.719 | 0.020 |
| 159 | 70618 | SENSE | 0.001 | 0.997 | 1.812 | 0.163 | 0.975 | 0.030 |
| 161 | 71306 | SENSE | 0.379 | 0.004 | 0.145 | 0.426 | 1.542 | 0.004 |
| 165 | 76533 | SENSE | 0.090 | 0.248 | 0.103 | 0.182 | 0.531 | 0.000 |
| 174 | 70978 | SENSE | 0.472 | 0.238 | 1.308 | 0.105 | 1.664 | 0.026 |
| 176 | 70977 | SENSE | 0.337 | 0.015 | 0.999 | 0.304 | 1.193 | 0.004 |
| 178 | 19762 | SENSE | 0.042 | 0.614 | −0.259 | 0.005 | 0.714 | 0.024 |
| 179 | 70938 | SENSE | 0.266 | 0.094 | 0.449 | 0.360 | 1.024 | 0.037 |
| 195 | 77605 | SENSE | 0.167 | 0.087 | 0.648 | 0.392 | 1.254 | 0.012 |
| 207 | 75453 | SENSE | −0.352 | 0.026 | 0.517 | 0.310 | 0.585 | 0.013 |
| 210 | 75467 | SENSE | 0.120 | 0.438 | −0.134 | 0.069 | 1.029 | 0.037 |
| 213 | 75470 | SENSE | 0.225 | 0.143 | −0.030 | 0.600 | 1.135 | 0.017 |
| 158 | 70450 | SENSE | 0.354 | 0.023 | 0.878 | 0.006 | 1.161 | 0.008 |

Transgenic plants comprising recombinant DNA expressing protein as set forth in SEQ ID NO: 122, 134, 144 and 193 showed enhanced heat stress tolerance by the second criterial as illustrated in Example 1L and 1M.

### D. Salt Stress Tolerance Screen

This example sets forth the high salinity stress screen to identify Arabidopsis plants transformed with the gene of interest that are tolerant to high levels of salt based on their rate of development, root growth and chlorophyll accumulation under high salt conditions.

T2 seeds were plated on glufosinate selection plates containing 90 mM NaCl and grown under standard light and temperature conditions. All seedlings used in the embodiments were grown at a temperature of 22° C. at day and 20° C. at night, a 16-hour photoperiod, an average light intensity of approximately 120 umol/m². On day 11, plants were measured for primary root length. After 3 more days of growth (day 14), plants were scored for transgenic status, primary root length, growth stage, visual color, and the seedlings were pooled for fresh weight measurement. A photograph of the whole plate was also taken on day 14.

The seedling weight and root length were analyzed as quantitative responses according to example 1M. The final growth stage at day 14 was scored as success if 50% of the plants reached 3 rosette leaves and size of leaves are greater than 1 mm (Boyes, D. C., et al., (2001), The Plant Cell 13, 1499/1510). The growth stage data was analyzed as a qualitative response according to example 1L. A list of recombinant DNA constructs that improve high salinity tolerance in transgenic plants illustrated in Table 6.

TABLE 6

| | | Root length | | Root length | | Growth stage | | Seedling weight | |
|---|---|---|---|---|---|---|---|---|---|
| PEP | | at day 11 | | at day 14 | | at day 14 | | at day 14 | |
| SEQ | Construct | Delta | | Delta | | Delta | | Delta | |
| ID | ID | mean | P-value | mean | P-value | mean | P-value | mean | P-value |
| 116 | 12155 | 0.207 | 0.258 | 0.297 | 0.020 | 0.691 | 0.256 | 0.622 | 0.012 |
| 141 | 18388 | 0.415 | 0.011 | 0.475 | 0.009 | 0.440 | 0.127 | 0.385 | 0.214 |
| 138 | 18446 | 0.120 | 0.385 | 0.120 | 0.097 | / | / | 0.490 | 0.042 |
| 133 | 18453 | 0.265 | 0.089 | 0.288 | 0.060 | 0.364 | 0.336 | 0.356 | 0.043 |
| 148 | 19191 | 0.024 | 0.383 | 0.104 | 0.019 | 0.210 | 0.466 | −0.197 | 0.034 |
| 162 | 70451 | 0.185 | 0.286 | 0.267 | 0.195 | 0.286 | 0.429 | 0.713 | 0.029 |
| 159 | 70618 | 0.504 | 0.017 | 0.535 | 0.046 | 0.358 | 0.403 | 1.101 | 0.010 |
| 145 | 70737 | 0.141 | 0.157 | 0.216 | 0.038 | 0.983 | 0.236 | 0.323 | 0.208 |
| 175 | 70940 | 0.668 | 0.007 | 0.672 | 0.004 | 3.597 | 0.012 | 1.201 | 0.001 |
| 173 | 70953 | 0.254 | 0.054 | 0.297 | 0.052 | 1.040 | 0.413 | 0.787 | 0.026 |
| 191 | 72046 | 0.236 | 0.010 | 0.168 | 0.000 | 0.502 | 0.184 | 0.215 | 0.177 |
| 137 | 73606 | 0.399 | 0.035 | 0.386 | 0.041 | 0.251 | 0.332 | 0.284 | 0.612 |
| 196 | 73978 | 0.413 | 0.076 | 0.327 | 0.016 | 0.269 | 0.423 | 0.451 | 0.073 |
| 126 | 75090 | 0.466 | 0.024 | 0.507 | 0.012 | 1.340 | 0.154 | 1.092 | 0.036 |
| 219 | 75545 | −0.274 | 0.077 | −0.105 | 0.314 | 0.197 | 0.320 | 0.468 | 0.018 |
| 228 | 77941 | 0.341 | 0.013 | 0.336 | 0.041 | 1.155 | 0.266 | 0.574 | 0.149 |

Transgenic plants comprising recombinant DNA expressing protein as set forth in SEQ ID NO: 117, 140, 146, 177, or 193 showed enhanced salt stress tolerance by the second criterial as illustrated in Example 1L and 1M.

### E. Polyethylene Glycol (PEG) Induced Osmotic Stress Tolerance Screen

There are numerous factors, which can influence seed germination and subsequent seedling growth, one being the availability of water. Genes, which can directly affect the success rate of germination and early seedling growth, are potentially useful agronomic traits for improving the to germination and growth of crop plants under drought stress. In this assay, PEG was used to induce osmotic stress on germinating transgenic lines of Arabidopsis thaliana seeds in order to screen for osmotically resistant seed lines.

T2 seeds were plated on BASTA selection plates containing 3% PEG and grown under standard light and temperature conditions. Seeds were plated on each plate containing 3% PEG, ½× MS salts, 1% phytagel, and 10 μg/ml glufosinate. Plates were placed at 4° C. for 3 days to stratify seeds. On day 11, plants were measured for primary root length. After 3 more days of growth, i.e., at day 14, plants were scored for transgenic status, primary root length, growth stage, visual color, and the seedlings were pooled for fresh weight measurement. A photograph of the whole plate was taken on day 14.

Seedling weight and root length were analyzed as quantitative responses according to example 1M. The final growth stage at day 14 was scored as success or failure based on whether the plants reached 3 rosette leaves and size of leaves are greater than 1 mm. The growth stage data was analyzed as a qualitative response according to example 1L. A list of recombinant DNA constructs that improve osmotic stress tolerance in transgenic plants illustrated in Table 7.

TABLE 7

| | | Root length | | Root length | | Growth stage | | Seedling weight | |
|---|---|---|---|---|---|---|---|---|---|
| PEP | | at day 11 | | at day 14 | | at day 14 | | at day 14 | |
| SEQ | Construct | Delta | | Delta | | Delta | | Delta | |
| ID | ID | mean | P-value | mean | P-value | mean | P-value | mean | P-value |
| 128 | 17524 | 0.468 | 0.016 | 0.441 | 0.026 | 4.000 | / | 0.845 | 0.004 |
| 135 | 18210 | 0.146 | 0.027 | 0.159 | 0.032 | −0.184 | 0.402 | −0.001 | 0.994 |
| 146 | 18315 | 0.275 | 0.025 | 0.207 | 0.005 | 1.235 | 0.468 | 0.744 | 0.058 |
| 152 | 19536 | 0.252 | 0.016 | 0.257 | 0.033 | 0.565 | 0.451 | 0.256 | 0.154 |
| 155 | 19614 | 0.271 | 0.028 | 0.138 | 0.237 | 4.000 | / | 0.325 | 0.043 |
| 171 | 19940 | 0.262 | 0.020 | 0.288 | 0.010 | 2.958 | 0.105 | 0.422 | 0.021 |
| 179 | 70938 | 0.303 | 0.099 | 0.256 | 0.258 | 4.000 | / | 0.593 | 0.038 |
| 168 | 70954 | 0.198 | 0.118 | 0.107 | 0.460 | 4.000 | / | 0.410 | 0.008 |
| 170 | 70960 | 0.332 | 0.019 | 0.350 | 0.064 | 4.000 | / | 0.399 | 0.009 |
| 174 | 70978 | 0.180 | 0.172 | 0.092 | 0.202 | 4.000 | / | 0.643 | 0.003 |
| 161 | 71306 | 0.284 | 0.104 | 0.150 | 0.277 | 4.000 | / | 0.646 | 0.041 |
| 192 | 72071 | 0.276 | 0.006 | 0.357 | 0.026 | 2.587 | 0.209 | −0.033 | 0.831 |
| 199 | 73493 | 0.129 | 0.171 | 0.142 | 0.014 | 1.802 | 0.291 | 0.221 | 0.002 |
| 139 | 75011 | 0.367 | 0.001 | 0.199 | 0.030 | 4.000 | / | 0.691 | 0.060 |
| 147 | 75080 | 0.162 | 0.001 | 0.107 | 0.014 | 1.396 | 0.068 | 0.347 | 0.013 |
| 206 | 75329 | 0.039 | 0.690 | 0.192 | 0.002 | 2.406 | 0.270 | 0.075 | 0.674 |
| 209 | 75430 | 0.105 | 0.134 | 0.294 | 0.038 | 1.284 | 0.485 | −0.160 | 0.075 |
| 153 | 76577 | 0.254 | 0.224 | 0.258 | 0.044 | 4.000 | / | 0.381 | 0.079 |
| 195 | 77605 | 0.340 | 0.150 | 0.445 | 0.095 | 2.635 | 0.061 | 0.685 | 0.036 |
| 120 | 78706 | 0.314 | 0.061 | 0.299 | 0.045 | 2.973 | 0.101 | 0.319 | 0.000 |

Transgenic plants comprising recombinant DNA expressing protein as set forth in SEQ ID NO: 118, 122, 126, 142, 173, 196, or 208 showed enhanced PEG osmotic stress tolerance by the second criterial as illustrated in Example 1L and 1M.

### F. Cold Shock Tolerance Screen

This example set forth a screen to identify Arabidopsis plants transformed with the genes of interest that are more tolerant to cold stress subjected during day 8 to day 28 after seed planting. During these crucial early stages, seedling growth and leaf area increase were measured to assess tolerance when Arabidopsis seedlings were exposed to low temperatures. Using this screen, genetic alterations can be found that enable plants to germinate and grow better than wild type plants under sudden exposure to low temperatures.

Eleven seedlings from T2 seeds of each transgenic line plus one control line were plated together on a plate containing ½× Gamborg Salts with 0.8 Phytagel™, 1% Phytagel, and 0.3% Sucrose. Plates were then oriented horizontally and stratified for three days at 4° C. At day three, plates were removed from stratification and exposed to standard conditions (16 hr photoperiod, 22° C. at day and 20° C. at night) until day 8. At day eight, plates were removed from standard conditions and exposed to cold shock conditions (24 hr photoperiod, 8° C. at both day and night) until the final day of the assay, i.e., day 28. Rosette areas were measured at day 8 and day 28, which were analyzed as quantitative responses according to example 1M. A list of recombinant nucleotides that improve cold shock stress tolerance in plants is illustrated in Table 8.

TABLE 8

| | | | | | Rosette area | | | |
|---|---|---|---|---|---|---|---|---|
| | | | Rosette area | | at day 28 | | Rosette area | |
| PEP | Con- | | at day 8 | | Risk | | difference | |
| SEQ | struct | Orien- | Delta | P- | score | P- | Delta | P- |
| ID | ID | tation | mean | value | mean | value | mean | value |
| 122 | 17520 | SENSE | 0.764 | 0.211 | 1.198 | 0.021 | 1.296 | 0.022 |
| 134 | 17913 | SENSE | 0.585 | 0.033 | 0.403 | 0.021 | 0.437 | 0.000 |
| 156 | 18204 | SENSE | 0.451 | 0.099 | 0.443 | 0.060 | 0.452 | 0.040 |
| 140 | 18507 | SENSE | 1.039 | 0.006 | 1.638 | 0.003 | 1.758 | 0.005 |
| 172 | 70921 | SENSE | 0.085 | 0.732 | 0.157 | 0.110 | 0.306 | 0.033 |
| 200 | 71228 | ANTI- | −0.254 | 0.340 | 0.677 | 0.002 | 1.033 | 0.002 |
| | | SENSE | | | | | | |
| 195 | 77605 | SENSE | 0.019 | 0.949 | 1.008 | 0.027 | 1.017 | 0.027 |

Transgenic plants comprising recombinant DNA expressing protein as set forth in SEQ ID NO: 128, 130, or 137 showed enhanced cold stress tolerance by the second criterial as illustrated in Example 1L and 1M.

### G. Cold Germination Tolerance Screen

This example sets forth a screen to identify Arabidopsis plants transformed with the genes of interests are resistant to cold stress based on their rate of development, root growth and chlorophyll accumulation under low temperature conditions.

T2 seeds were plated and all seedlings used in the embodiments were grown at 8° C. Seeds were first surface disinfested using chlorine gas and then seeded on assay plates containing an aqueous solution of ½× Gamborg's B/5 Basal Salt Mixture (Sigma/Aldrich Corp., St. Louis, Mo., USA G/5788), 1% Phytagel™ (Sigma-Aldrich, P-8169), and 10 ug/ml glufosinate with the final pH adjusted to 5.8 using KOH. Test plates were held vertically for 28 days at a constant temperature of 8° C., a photoperiod of 16 hr, and average light intensity of approximately 100 umol/m²/s. At 28 days post plating, root length was measured, growth stage was observed, the visual color was assessed, and a whole plate photograph was taken.

The root length at day 28 was analyzed as a quantitative response according to example 1M. The growth stage at day 7 was analyzed as a qualitative response according to example 1L. A list of recombinant DNA constructs that improve cold stress tolerance in transgenic plants illustrated in Table 9.

TABLE 9

| | | | | Root length | | Growth stage | |
|---|---|---|---|---|---|---|---|
| PEP | Con- | | | at day 28 | | at day 28 | |
| SEQ | struct | Nomination | Orien- | Delta | P- | Delta | P- |
| ID | ID | ID | tation | mean | value | mean | value |
| 154 | 11113 | CGPG353 | ANTI- | 0.180 | 0.281 | 4.000 | 0.000 |
| | | | SENSE | | | | |
| 116 | 12155 | CGPG1135 | ANTI- | −0.110 | 0.202 | 4.000 | 0.000 |
| | | | SENSE | | | | |
| 125 | 17468 | CGPG2602 | SENSE | 0.251 | 0.075 | 4.000 | 0.000 |
| 144 | 18411 | CGPG2996 | SENSE | −0.016 | 0.839 | 4.000 | 0.000 |
| 133 | 18453 | CGPG2759 | SENSE | 0.177 | 0.247 | 4.000 | 0.000 |
| 142 | 18540 | CGPG2967 | SENSE | −0.096 | 0.749 | 4.000 | 0.000 |
| 143 | 19653 | CGPG2983 | SENSE | −0.024 | 0.911 | 4.000 | 0.000 |
| 169 | 19742 | CGPG3977 | SENSE | 0.135 | 0.292 | 4.000 | 0.000 |
| 163 | 70453 | CGPG3799 | SENSE | 0.039 | 0.660 | 4.000 | 0.000 |
| 172 | 70921 | CGPG4068 | SENSE | 0.125 | 0.029 | 4.000 | 0.000 |
| 179 | 70938 | CGPG4193 | SENSE | 0.008 | 0.955 | 4.000 | 0.000 |
| 177 | 70993 | CGPG4167 | SENSE | 0.183 | 0.027 | 4.000 | 0.000 |
| 164 | 71311 | CGPG3822 | SENSE | 0.531 | 0.008 | 4.000 | 0.000 |
| 191 | 72046 | CGPG5284 | SENSE | 0.055 | 0.721 | 4.000 | 0.000 |
| 123 | 72672 | CGPG2591 | SENSE | 0.443 | 0.101 | 4.000 | 0.000 |
| 130 | 72977 | CGPG2710 | SENSE | −0.315 | 0.347 | 4.000 | 0.000 |
| 157 | 73614 | CGPG3751 | SENSE | 0.939 | 0.009 | 4.000 | 0.000 |
| 201 | 74889 | CGPG7334 | SENSE | −0.144 | 0.648 | 4.000 | 0.000 |
| 203 | 74891 | CGPG7350 | SENSE | −0.042 | 0.860 | 4.000 | 0.000 |
| 210 | 75467 | CGPG7615 | SENSE | 0.167 | 0.308 | 4.000 | 0.000 |
| 211 | 77371 | CGPG8175 | SENSE | 0.092 | 0.530 | 4.000 | 0.000 |

Transgenic plants comprising recombinant DNA expressing protein as set forth in SEQ ID NO: 128, 130, or 137 showed enhanced cold stress tolerance by the second criterial as illustrated in Example 1L and 1M.

### H. Shade Tolerance Screen

Plants undergo a characteristic morphological response in shade that includes the elongation of the petiole, a change in the leaf angle, and a reduction in chlorophyll content. While these changes can confer a competitive advantage to individuals, in a monoculture the shade avoidance response is thought to reduce the overall biomass of the population. Thus, genetic alterations that prevent the shade avoidance response can be associated with higher yields. Genes that favor growth under low light conditions can also promote yield, as inadequate light levels frequently limit yield. This protocol describes a screen to look for Arabidopsis plants that show an attenuated shade avoidance response and/or grow better than control plants under low light intensity. Of particular interest, we were looking for plants that didn't extend their petiole length, had an increase in seedling weight relative to the reference and had leaves that were more close to parallel with the plate surface.

T2 seeds were plated on glufosinate selection plates with ½ MS medium. Seeds were sown on ½× MS salts, 1% Phytagel, 10 ug/ml BASTA. Plants were grown on vertical plates at a temperature of 22° C. at day, 20° C. at night and under low light (approximately 30 uE/m²/s, far/red ratio (655/665/725/735) ˜0.35 using PLAQ lights with GAM color filter #680). Twenty-three days after seedlings were sown, measurements were recorded including seedling status, number of rosette leaves, status of flower bud, petiole leaf angle, petiole length, and pooled fresh weights. A digital image of the whole plate was taken on the measurement day. Seedling weight and petiole length were analyzed as quantitative responses according to example 1M. The number of rosette leaves, flowering bud formation and leaf angel were analyzed as qualitative responses according to example 1L.

A list of recombinant DNA constructs that improve shade tolerance in plants illustrated in Table 10.

TABLE 10

| | | | | Seedling | | Petiole | |
|---|---|---|---|---|---|---|---|
| | | | | weight at | | length at | |
| PEP | Con- | Nomi- | | day 23 | | day 23 | |
| SEQ | struct | nation | Orien- | Delta | P- | Delta | P- |
| ID | ID | ID | tation | mean | value | mean | value |
| 160 | 70458 | CGPG3757 | SENSE | −0.379 | 0.068 | −0.295 | 0.091 |
| 185 | 70769 | CGPG4622 | SENSE | −0.447 | 0.155 | −0.350 | 0.074 |
| 186 | 71643 | CGPG4700 | SENSE | 0.010 | 0.950 | −0.202 | 0.047 |
| 127 | 72018 | CGPG2651 | SENSE | −0.715 | 0.047 | −0.628 | 0.065 |
| 190 | 72034 | CGPG5283 | SENSE | −0.709 | 0.065 | −1.001 | 0.042 |
| 129 | 72078 | CGPG2708 | SENSE | −1.404 | 0.006 | −1.055 | 0.045 |
| 194 | 72108 | CGPG5322 | SENSE | −0.327 | 0.059 | −0.278 | 0.083 |
| 197 | 73032 | CGPG5806 | SENSE | −1.540 | 0.013 | −1.286 | 0.010 |
| 181 | 73216 | CGPG4528 | SENSE | −0.723 | 0.016 | −0.476 | 0.041 |
| 183 | 74227 | CGPG4560 | SENSE | −0.961 | 0.004 | −0.725 | 0.063 |
| 205 | 75340 | CGPG7493 | SENSE | −1.326 | 0.061 | −1.053 | 0.075 |
| 209 | 75430 | CGPG7604 | SENSE | −0.643 | 0.086 | −0.372 | 0.092 |
| 208 | 75477 | CGPG7600 | SENSE | −0.813 | 0.040 | −0.986 | 0.034 |
| 220 | 75581 | CGPG7734 | SENSE | −0.679 | 0.018 | −0.616 | 0.070 |
| 222 | 75658 | CGPG7836 | SENSE | −0.787 | 0.003 | −0.544 | 0.034 |
| 223 | 75738 | CGPG7867 | SENSE | −0.269 | 0.511 | −0.436 | 0.038 |
| 224 | 77356 | CGPG8105 | SENSE | −1.669 | 0.003 | −2.412 | 0.055 |
| 215 | 77822 | CGPG7691 | SENSE | −0.738 | 0.112 | −0.860 | 0.027 |
| 180 | 78653 | CGPG4210 | SENSE | −0.578 | 0.021 | −0.702 | 0.068 |
| 132 | 78979 | CGPG2736 | SENSE | −1.116 | 0.023 | −0.736 | 0.069 |

For “seeding weight”, if p<0.05 and delta or risk score mean >0, the transgenic plants showed statistically significant trait enhancement as compared to the reference. If p<0.2 and delta or risk score mean >0, the transgenic plants showed a trend of trait enhancement as compared to the reference with p<0.2.

For “petiole length”, if p<0.05 and delta <0, the transgenic plants showed statistically significant trait enhancement as compared to the reference. If p<0.2 and delta <0, the transgenic plants showed a trend of trait enhancement as compared to the reference.

Transgenic plants comprising recombinant DNA expressing protein as set forth in SEQ ID NO: 128, 184, or 195 showed enhanced tolerance to shade or low light condition by the second criterial as illustrated in Example 1L and 1M.

### I. Early Plant Growth and Development Screen

This example sets forth a plate based phenotypic analysis platform for the rapid detection of phenotypes that are evident during the first two weeks of growth. In this screen, we were looking for genes that confer advantages in the processes of germination, seedling vigor, root growth and root morphology under non-stressed growth conditions to plants. The transgenic plants with advantages in seedling growth and development were determined by the seedling weight and root length at day 14 after seed planting.

T2 seeds were plated on glufosinate selection plates and grown under standard conditions (˜100 uE/m²/s, 16 h photoperiod, 22° C. at day, 20° C. at night). Seeds were stratified for 3 days at 4° C. Seedlings were grown vertically (at a temperature of 22° C. at day 20° C. at night). Observations were taken on day 10 and day 14. Both seedling weight and root length at day 14 were analyzed as quantitative responses according to example 1M.

A list recombinant DNA constructs that improve early plant growth and development illustrated in Table 11.

TABLE 11

| | | | | Root length | | Root length | | Seedling weight | |
|---|---|---|---|---|---|---|---|---|---|
| PEP | | | | at day 10 | | at day 14 | | at day 14 | |
| SEQ | Construct | Nomination | | Delta | | Delta | | Delta | |
| ID | ID | ID | Orientation | mean | P-value | mean | P-value | mean | P-value |
| 151 | 18434 | CGPG3454 | SENSE | 0.188 | 0.069 | 0.031 | 0.585 | 0.040 | 0.714 |
| 133 | 18453 | CGPG2759 | SENSE | 0.199 | 0.149 | 0.222 | 0.070 | 0.061 | 0.605 |
| 178 | 19762 | CGPG4176 | SENSE | 0.226 | 0.009 | 0.133 | 0.031 | 0.045 | 0.835 |
| 173 | 70953 | CGPG4105 | SENSE | 0.347 | 0.032 | 0.309 | 0.052 | 0.275 | 0.273 |
| 126 | 75090 | CGPG2642 | SENSE | 0.316 | 0.020 | 0.146 | 0.095 | 0.459 | 0.073 |
| 211 | 75457 | CGPG7630 | SENSE | / | / | / | / | 0.373 | 0.019 |
| 213 | 75470 | CGPG7639 | SENSE | 0.186 | 0.069 | 0.128 | 0.058 | 0.318 | 0.039 |
| 216 | 75505 | CGPG7696 | SENSE | 0.135 | 0.018 | 0.051 | 0.567 | 0.079 | 0.564 |
| 165 | 76533 | CGPG3890 | SENSE | 0.225 | 0.075 | 0.135 | 0.112 | −0.119 | 0.531 |

Transgenic plants comprising recombinant DNA expressing a protein as set forth in SEQ ID NO: 122, 142, 179, 191 or 195 showed improved early plant growth and development by the second criterial as illustrated in Example 1L and 1M.

### J. Late Plant Growth and Development Screen

This example sets forth a soil based phenotypic platform to identify genes that confer advantages in the processes of leaf development, flowering production and seed maturity to plants.

Arabidopsis plants were grown on a commercial potting mixture (Metro Mix 360, Scotts Co., Marysville, Ohio) consisting of 30-40% medium grade horticultural vermiculite, 35-55% sphagnum peat moss, 10-20% processed bark ash, 1-15% pine bark and a starter nutrient charge. Soil was supplemented with Osmocote time-release fertilizer at a rate of 30 mg/ft³. T2 seeds were imbibed in 1% agarose solution for 3 days at 4° C. and then sown at a density of ˜5 per 2 ½″ pot. Thirty-two pots were ordered in a 4 by 8 grid in standard greenhouse flat. Plants were grown in environmentally controlled rooms under a 16 h day length with an average light intensity of ˜200 μmoles/m²/s. Day and night temperature set points were 22° C. and 20° C., respectively. Humidity was maintained at 65%. Plants were watered by sub-irrigation every two days on average until mid-flowering, at which point the plants were watered daily until flowering was complete.

Application of the herbicide glufosinate was performed to select T2 individuals containing the target transgene. A single application of glufosinate was applied when the first true leaves were visible. Each pot was thinned to leave a single glufosinate-resistant seedling ˜3 days after the selection was applied.

The rosette radius was measured at day 25. The silique length was measured at day 40. The plant parts were harvested at day 49 for dry weight measurements if flowering production was stopped. Otherwise, the dry weights of rosette and silique were carried out at day 53. The seeds were harvested at day 58. All measurements were analyzed as quantitative responses according to example 1M.

A list of recombinant DNA constructs that improve late plant growth and development illustrated in Table 12.

TABLE 12

| | | Rosette dry weight | | Rosette radius | | Seed net dry weight | | Silique dry weight | | Silique length | |
|---|---|---|---|---|---|---|---|---|---|---|---|
| PEP | | at day 53 | | at day 25 | | at day 62 | | at day 53 | | at day 40 | |
| SEQ | Construct | Delta | | Delta | | Delta | | Delta | | Delta | |
| ID | ID | mean | P-value | mean | P-value | mean | P-value | mean | P-value | mean | P-value |
| 121 | 72666 | 0.194 | 0.032 | / | / | 0.111 | 0.267 | 0.073 | 0.524 | −0.104 | 0.078 |
| 150 | 18433 | −0.363 | 0.222 | 0.169 | 0.006 | 0.357 | 0.017 | / | / | 0.099 | 0.011 |
| 166 | 70955 | −0.137 | 0.052 | 0.298 | 0.003 | 0.951 | 0.022 | 0.163 | 0.096 | −0.001 | 0.930 |
| 161 | 71306 | −0.058 | 0.556 | 0.043 | 0.352 | 1.522 | 0.009 | 0.120 | 0.011 | 0.031 | 0.766 |
| 211 | 75457 | −0.147 | 0.153 | 0.294 | 0.055 | 1.099 | 0.014 | 0.496 | 0.060 | −0.217 | 0.052 |
| 212 | 75469 | 0.305 | 0.075 | 0.267 | 0.020 | 1.447 | 0.015 | 0.322 | 0.038 | −0.105 | 0.205 |
| 218 | 75542 | 1.026 | 0.018 | 0.152 | 0.406 | −0.166 | 0.465 | / | / | −0.265 | 0.174 |
| 223 | 75738 | −0.654 | 0.102 | 0.064 | 0.363 | 0.774 | 0.000 | 0.293 | 0.170 | −0.095 | 0.093 |

### K. Limited Nitrogen Tolerance Screen

Under low nitrogen conditions, Arabidopsis seedlings become chlorotic and have less biomass. This example sets forth the limited nitrogen tolerance screen to identify Arabidopsis plants transformed with the gene of interest that are altered in their ability to accumulate biomass and/or retain chlorophyll under low nitrogen condition.

T2 seeds were plated on glufosinate selection plates containing 0.5× N-Free Hoagland's T 0.1 mM NH₄NO₃ T 0.1% sucrose T 1% phytagel media and grown under standard light and temperature conditions. At 12 days of growth, plants were scored for seedling status (i.e., viable or non-viable) and root length. After 21 days of growth, plants were scored for BASTA resistance, visual color, seedling weight, number of green leaves, number of rosette leaves, root length and formation of flowering buds. A photograph of each plant was also taken at this time point.

The seedling weight and root length were analyzed as quantitative responses according to example 1M. The number green leaves, the number of rosette leaves and the flowerbud formation were analyzed as qualitative responses according to example 1L. The leaf color raw data were collected on each plant as the percentages of five color elements (Green, DarkGreen, LightGreen, RedPurple, YellowChlorotic) using a computer imaging system. A statistical logistic regression model was developed to predict an overall value based on five colors for each plant.

A list of recombinant DNA constructs that improve low nitrogen availability tolerance in plants illustrated in Table 13.

TABLE 13

| | | | | | Leaf color | | | |
|---|---|---|---|---|---|---|---|---|
| | | | Root length | | at day 21 | | Rosette weight | |
| PEP | | | at day 21 | | Risk | | at day 21 | |
| SEQ | Construct | | Delta | | score | | Delta | |
| ID | ID | Orientation | mean | P-value | mean | P-value | mean | P-value |
| 115 | 10177 | ANTI- | −0.556 | 0.008 | 3.633 | 0.013 | 0.199 | 0.056 |
| | | SENSE | | | | | | |
| 198 | 11157 | SENSE | −0.072 | 0.607 | 0.928 | 0.617 | 0.139 | 0.051 |
| 118 | 11873 | ANTI- | −0.327 | 0.037 | 3.065 | 0.018 | −0.013 | 0.822 |
| | | SENSE | | | | | | |
| 117 | 12233 | SENSE | / | / | 2.183 | 0.087 | 0.066 | 0.243 |
| 188 | 12325 | ANTI- | −0.366 | 0.100 | 1.462 | 0.027 | −0.037 | 0.588 |
| | | SENSE | | | | | | |
| 131 | 17445 | SENSE | / | / | 5.181 | 0.061 | −0.175 | 0.208 |
| 149 | 18431 | SENSE | −0.466 | 0.023 | 3.868 | 0.059 | −0.021 | 0.433 |
| 184 | 70678 | SENSE | −0.084 | 0.375 | 1.052 | 0.035 | 0.161 | 0.037 |
| 182 | 71139 | SENSE | −0.218 | 0.169 | −3.163 | 0.020 | 0.490 | 0.018 |
| 186 | 71643 | SENSE | 0.028 | 0.495 | −2.371 | 0.050 | 0.267 | 0.046 |
| 193 | 72116 | SENSE | −0.322 | 0.074 | −0.666 | 0.093 | 0.491 | 0.023 |
| 202 | 74854 | SENSE | / | / | 2.673 | 0.015 | −0.051 | 0.527 |
| 203 | 74891 | SENSE | −0.403 | 0.033 | 1.721 | 0.022 | 0.074 | 0.252 |
| 204 | 75314 | SENSE | −0.467 | 0.219 | 1.983 | 0.023 | −0.160 | 0.117 |
| 209 | 75430 | SENSE | −0.429 | 0.047 | 5.351 | 0.011 | −0.097 | 0.352 |
| 207 | 75453 | SENSE | 0.063 | 0.684 | −1.743 | 0.341 | 0.189 | 0.031 |
| 221 | 75524 | SENSE | −0.071 | 0.343 | 1.084 | 0.049 | −0.089 | 0.113 |
| 218 | 75542 | SENSE | −1.768 | 0.067 | 3.388 | 0.029 | −0.617 | 0.132 |
| 217 | 75589 | SENSE | −0.079 | 0.443 | 1.552 | 0.008 | 0.011 | 0.929 |
| 226 | 77363 | SENSE | / | / | 3.460 | 0.065 | −0.212 | 0.086 |
| 180 | 78653 | SENSE | 0.277 | 0.214 | 2.659 | 0.132 | 0.203 | 0.006 |
| 136 | 78710 | SENSE | / | / | 3.948 | 0.007 | 0.008 | 0.935 |
| 124 | 78977 | SENSE | / | / | 4.985 | 0.015 | 0.137 | 0.118 |
| 132 | 78979 | SENSE | −0.689 | 0.220 | 4.517 | 0.013 | −0.617 | 0.002 |

For rosette weight, if p<0.05 and delta or risk score mean >0, the transgenic plants showed statistically significant trait enhancement as compared to the reference. If p<0.2 and delta or risk score mean >0, the transgenic plants showed a trend of trait enhancement as compared to the reference with p<0.2. For root length, if p<0.05, the transgenic plants showed statistically significant trait enhancement as compared to the reference. If p<0.2, the transgenic plants showed a trend of trait enhancement as compared to the reference.

### L. Statistic Analysis for Qualitative Responses

A list of responses that were analyzed as qualitative responses illustrated in Table 14.

TABLE 14

| response | Screen | categories (success vs. failure) |
|---|---|---|
| Wilting response Risk | Soil drought tolerance screen | non-wilted vs. wilted |
| Score | | |
| growth stage at day 14 | heat stress tolerance screen | 50% of plants reach stage 1.03 vs. |
| | | not |
| growth stage at day 14 | salt stress tolerance screen | 50% of plants reach stage 1.03 vs. |
| | | not |
| growth stage at day 14 | PEG induced osmotic stress tolerance | 50% of plants reach stage 1.03 vs. |
| | screen | not |
| growth stage at day 7 | cold germination tolerance screen | 50% of plants reach stage 0.5 vs. not |
| number of rosette leaves | Shade tolerance screen | 5 leaves appeared vs. not |
| at day 23 | | |
| Flower bud formation at | Shade tolerance screen | flower buds appear vs. not |
| day 23 | | |
| leaf angle at day 23 | Shade tolerance screen | >60 degree vs. <60 degree |
| number of green leaves at | limited nitrogen tolerance screen | 6 or 7 leaves appeared vs. not |
| day 21 | | |
| number of rosette leaves | limited nitrogen tolerance screen | 6 or 7 leaves appeared vs. not |
| at day 21 | | |
| Flower bud formation at | limited nitrogen tolerance screen | flower buds appear vs. not |
| day 21 | | |

Plants were grouped into transgenic and reference groups and were scored as success or failure according to Table 14. First, the risk (R) was calculated, which is the proportion of plants that were scored as of failure plants within the group. Then the relative risk (RR) was calculated as the ratio of R (transgenic) to R (reference). Risk score (RS) was calculated as −log₂^{RR}. Two criteria were used to determine a transgenic with enhanced trait(s). Transgenic plants comprising recombinant DNA disclosed herein showed trait enhancement according to either or both of the two criteria.

For the first criteria, the risk scores from multiple events of the transgene of interest were evaluated for statistical significance by t-test using SAS statistical software (SAS 9, SAS/STAT User's Guide, SAS Institute Inc., Cary, N.C., USA). RS with a value greater than 0 indicates that the transgenic plants perform better than the reference. RS with a value less than 0 indicates that the transgenic plants perform worse than the reference. The RS with a value equal to 0 indicates that the performance of the transgenic plants and the reference don't show any difference. If p<0.05 and risk score mean >0, the transgenic plants showed statistically significant trait enhancement as compared to the reference. If p<0.2 and risk score mean >0, the transgenic plants showed a trend of trait enhancement as compared to the reference.

For the second criteria, the RS from each event was evaluated for statistical significance by t-test using SAS statistical software (SAS 9, SAS/STAT User's Guide, SAS Institute Inc, Cary, N.C., USA). The RS with a value greater than 0 indicates that the transgenic plants from this event performs better than the reference. The RS with a value less than 0 indicates that the transgenic plants from this event perform worse than the reference. The RS with a value equal to 0 indicates that the performance of the transgenic plants from this event and the reference don't show any difference. If p<0.05 and risk score mean >0, the transgenic plants from this event showed statistically significant trait enhancement as compared to the reference. If p<0.2 and risk score mean >0, the transgenic plants showed a trend of trait enhancement as compared to the reference.

If two or more events of the transgene of interest showed improvement in the same response, the transgene was deemed to show trait enhancement.

### M. Statistic Analysis for Quantitative Responses

A list of responses that were analyzed as quantitative responses illustrated in Table 15.

TABLE 15

| response | screen |
|---|---|
| seed yield | Soil drought stress tolerance screen |
| seedling weight at day 14 | heat stress tolerance screen |
| root length at day 14 | heat stress tolerance screen |
| seedling weight at day 14 | salt stress tolerance screen |
| root length at day 14 | salt stress tolerance screen |
| root length at day 11 | salt stress tolerance screen |
| seedling weight at day 14 | PEG induced osmotic stress tolerance screen |
| root length at day 11 | PEG induced osmotic stress tolerance screen |
| root length at day 14 | PEG induced osmotic stress tolerance screen |
| rosette area at day 8 | cold shock tolerance screen |
| rosette area at day28 | cold shock tolerance screen |
| difference in rosette area | cold shock tolerance screen |
| from day 8 to day 28 | |
| root length at day 28 | cold germination tolerance screen |
| seedling weight at day 23 | Shade tolerance screen |
| petiole length at day 23 | Shade tolerance screen |
| root length at day 14 | Early plant growth and development screen |
| Seedling weight at day14 | Early plant growth and development screen |
| Rosette dry weight at day 53 | Late plant growth and development screen |
| rosette radius at day 25 | Late plant growth and development screen |
| seed dry weight at day 58 | Late plant growth and development screen |
| silique dry weight at day 53 | Late plant growth and development screen |
| silique length at day 40 | Late plant growth and development screen |
| Seedling weight at day 21 | Limited nitrogen tolerance screen |
| Root length at day 21 | Limited nitrogen tolerance screen |

The measurements (M) of each plant were transformed by log2 calculation. The Delta was calculated as log₂M(transgenic)−log₂M(reference). Two criteria were used to determine trait enhancement. A transgene of interest could show trait enhancement according to either or both of the two criteria. The measurements (M) of each plant were transformed by log2 calculation. The Delta was calculated as log₂M(transgenic)−log2M(reference). If the measured response was Petiole Length for the Low Light assay, Delta was subsequently multiplied by -1, to account for the fact that a shorter petiole length is considered an indication of trait enhancement.

For the first criteria, the Deltas from multiple events of the transgene of interest were evaluated for statistical significance by t-test using SAS statistical software (SAS 9, SAS/STAT User's Guide, SAS Institute Inc, Cary, N.C., USA). Delta with a value greater than 0 indicates that the transgenic plants perform better than the reference. Delta with a value less than 0 indicates that the transgenic plants perform worse than the reference. The Delta with a value equal to 0 indicates that the performance of the transgenic plants and the reference don't show any difference. If p<0.05 and risk score mean >0, the transgenic plants showed statistically significant trait enhancement as compared to the reference. If p<0.2 and risk score mean >0, the transgenic plants showed a trend of trait enhancement as compared to the reference.

For the second criteria, the delta from each event was evaluated for statistical significance by t-test using SAS statistical software (SAS 9, SAS/STAT User's Guide, SAS Institute Inc., Cary, N.C., USA). The Delta with a value greater than 0 indicates that the transgenic plants from this event performs better than the reference. The Delta with a value less than 0 indicates that the transgenic plants from this event perform worse than the reference. The Delta with a value equal to 0 indicates that the performance of the transgenic plants from this event and the reference don't show any difference. If p<0.05 and delta mean >0, the transgenic plants from this event showed statistically significant trait improvement as compared to the reference. If p<0.2 and delta mean >0, the transgenic plants showed a trend of trait enhancement as compared to the reference. If two or more events of the transgene of interest showed enhancement in the same response, the transgene was deemed to show trait improvement.

### Example 2

### Identification of Homologs

A BLAST searchable “All Protein Database” is constructed of known protein sequences using a proprietary sequence database and the National Center for Biotechnology Information (NCBI) non-redundant amino acid database (nr.aa). For each organism from which a DNA sequence provided herein was obtained, an “Organism Protein Database” is constructed of known protein sequences of the organism; the Organism Protein Database is a subset of the All Protein Database based on the NCBI taxonomy ID for the organism.

The All Protein Database is queried using amino acid sequence of cognate protein for gene DNA used in trait-improving recombinant DNA, i.e., sequences of SEQ ID NO: 115 through SEQ ID NO: 228 using “blastp” with E-value cutoff of le-8. Up to 1000 top hits were kept, and separated by organism names. For each organism other than that of the query sequence, a list is kept for hits from the query organism itself with a more significant E-value than the best hit of the organism. The list contains likely duplicated genes, and is referred to as the Core List. Another list was kept for all the hits from each organism, sorted by E-value, and referred to as the Hit List.

The Organism Protein Database is queried using amino acid sequences of SEQ ID NO: 115 through SEQ ID NO: 228 using “blastp” with E-value cutoff of le-4. Up to 1000 top hits are kept. A BLAST searchable database is constructed based on these hits, and is referred to as “SubDB”. SubDB was queried with each sequence in the Hit List using “blastp” with E-value cutoff of le-8. The hit with the best E-value is compared with the Core List from the corresponding organism. The hit is deemed a likely ortholog if it belongs to the Core List, otherwise it is deemed not a likely ortholog and there is no further search of sequences in the Hit List for the same organism. Likely orthologs from a large number of distinct organisms were identified and are reported by amino acid sequences of SEQ ID NO: 229 to SEQ ID NO: 4815. These orthologs are reported in Tables 16 as homologs to the proteins cognate to genes used in trait-improving recombinant DNA.

TABLE 16

| | | | | | | | | | | | | |
|---|---|---|---|---|---|---|---|---|---|---|---|---|
| 115: | 1365 | 1361 | 2178 | 2823 | 2234 | 2825 | 1854 | 245 | 1518 | 2928 | 3573 | 2922 |
| | 955 | 1930 | 1915 | 2795 | 467 | 3944 | 3288 | 4383 | 4338 | 3604 | 2927 | 345 |
| | 4267 | 4265 | 2723 | 3383 | 2779 | 4284 | 4571 | 2432 | 316 | 310 | 3031 | 2099 |
| | 725 | 762 | 442 | 2727 | 4758 | 3997 | 4738 | 2662 | 313 | 405 | 1748 | 3751 |
| | 4132 | 3483 | 1398 | 3343 | 2900 | 397 | 1254 | 2642 | 1508 | 4249 | 2573 | 4178 |
| | 4424 | 593 | 738 | 595 | 597 | 1798 | 552 | 923 | 2444 | 2389 | 2396 | 2740 |
| | 607 | 1462 | | | | | | | | | | |
| 116: | 4081 | 1259 | 3766 | 3192 | 503 | 1192 | 4791 | 698 | 1068 | 3223 | 785 | 4102 |
| 117: | 887 | 4054 | 4545 | 665 | 4519 | 3562 | 3980 | 3020 | 4522 | 1726 | 3155 | 657 |
| | 1358 | 4086 | 3966 | 2065 | 1005 | 2184 | 4022 | 1570 | 1032 | | | |
| 118: | 1513 | 2965 | 4541 | 3048 | 3971 | 3972 | 1774 | 477 | 4041 | 2003 | 3299 | 3663 |
| | 1445 | 2798 | 1168 | 4511 | 1889 | 4626 | 3152 | | | | | |
| 119: | 2925 | 2260 | 3866 | 4714 | 2527 | 3711 | 4566 | 2368 | 1960 | 3655 | 1894 | 2960 |
| | 4271 | 2098 | 2639 | 539 | 1297 | 4222 | 4224 | 2811 | 1914 | 516 | 3455 | 3807 |
| | 1505 | 832 | 1843 | 1740 | 4435 | 240 | 1919 | 4235 | 1867 | 3639 | 3240 | 1861 |
| | 2797 | 244 | 4414 | 4612 | 2951 | 732 | 1805 | 3571 | 3838 | 2038 | 3559 | 1002 |
| 120: | 2141 | 365 | 2225 | 1364 | 876 | 4283 | 2146 | 4286 | 1974 | 1127 | 3389 | 1465 |
| | 556 | 2801 | 4693 | 273 | 2062 | 360 | 1124 | 320 | 341 | 338 | 335 | 292 |
| | 289 | 285 | 283 | 3419 | 3557 | | | | | | | |
| 121: | 642 | 2784 | 2160 | 3289 | 2668 | 3254 | 4128 | 425 | 1248 | 2013 | 4371 | 4598 |
| | 2019 | 1144 | 820 | 1036 | 4619 | 3449 | | | | | | |
| 122: | 3177 | 4253 | 4257 | 2808 | 2789 | 2433 | 1394 | 1339 | 1333 | 1336 | 1335 | 4636 |
| | 2178 | 4187 | 811 | 243 | 2305 | 2708 | 2702 | 4614 | 2635 | 2640 | 2795 | 2927 |
| | 3411 | 3415 | 723 | 2829 | 2111 | 2103 | 2653 | 2628 | 2625 | 948 | 540 | 4131 |
| | 4147 | 4228 | 4244 | 4213 | 305 | 303 | 1255 | 323 | 2389 | 1784 | | |
| 123: | 3891 | 574 | 1849 | 1832 | 4018 | 3244 | 3777 | 3780 | 291 | 4618 | 994 | 965 |
| | 1028 | 961 | 943 | 939 | 1568 | 1122 | 1119 | 1095 | 1025 | 1010 | 1006 | 1044 |
| | 1077 | 1043 | 992 | 989 | 3121 | 356 | 354 | 1372 | 3106 | 993 | 390 | 3391 |
| | 4210 | 4252 | 1630 | 1813 | 3431 | 3636 | 3161 | 3585 | | | | |
| 124: | 2583 | 2651 | 3877 | 2863 | 4243 | 1233 | 2941 | 3494 | 2914 | 3154 | 4793 | 454 |
| | 3675 | 937 | 380 | 3067 | 4551 | 4057 | 1220 | 4251 | 4588 | 3970 | 4474 | 2864 |
| | 4032 | 2654 | 1455 | 3380 | 4351 | 2633 | 4809 | 2410 | 421 | 1007 | 2802 | 2387 |
| 125: | 2178 | 4315 | 811 | 815 | 2370 | 247 | 1178 | 1518 | 2331 | 2795 | 478 | 3089 |
| | 2222 | 2226 | 2247 | 2250 | 3604 | 2927 | 2966 | 3852 | 1626 | 4068 | 2332 | 1594 |
| | 4814 | 2369 | 2367 | 3140 | 2829 | 1586 | 4070 | 3113 | 899 | 921 | 2233 | 1065 |
| | 1613 | 2747 | 4527 | 2417 | 1667 | 1670 | 4249 | 624 | 745 | 2445 | 2929 | 2787 |
| | 923 | 2148 | 792 | 323 | 3071 | 2348 | 3810 | 2444 | 2389 | 2740 | 3404 | |
| 126: | 3322 | 1820 | 4486 | 4040 | 695 | 3253 | 3887 | 3307 | 3943 | 3835 | 3992 | 4014 |
| | 3362 | 2650 | 2706 | 3574 | 777 | 1407 | 1175 | 3850 | 4406 | 2270 | 4603 | 2885 |
| | 947 | 3776 | 827 | 1939 | 1058 | 545 | 466 | 4658 | 1812 | 2011 | 3736 | 2139 |
| | 3057 | 1551 | 1230 | 2469 | 1828 | 3750 | 1397 | 2563 | 1191 | 3441 | 1278 | 3531 |
| | 2403 | 420 | 2084 | 4164 | 2053 | 3599 | | | | | | |
| 127: | 2583 | 3877 | 4683 | 1233 | 4251 | 1220 | 4588 | 4474 | 3970 | 2864 | 4032 | 2654 |
| | 4351 | 3380 | 1455 | 4809 | 2633 | 2410 | 421 | 1007 | 4806 | 3818 | | |
| 128: | 401 | 2840 | 3369 | 2530 | 2805 | 4475 | 324 | 1347 | 2549 | 2063 | 3326 | 4676 |
| 129: | 4020 | 703 | 2462 | 2488 | 1797 | 991 | 4489 | 4582 | 1351 | 931 | 4647 | 2200 |
| | 3700 | 2532 | 4796 | 2240 | 2407 | 2104 | 952 | 1081 | 3165 | 3246 | | |
| 130: | 3424 | 2061 | 3913 | 259 | 3775 | 1236 | 4394 | 4028 | 543 | 603 | 1767 | 2980 |
| | 3687 | 998 | 359 | 2842 | 3462 | 1684 | 1180 | 1418 | 2905 | 450 | 3132 | 1803 |
| | 3360 | 638 | 3859 | 4066 | 1291 | 3917 | 1295 | 272 | | | | |
| 131: | 4813 | 1365 | 1361 | 2178 | 2823 | 4187 | 2234 | 2825 | 811 | 815 | 1518 | 2050 |
| | 2640 | 2635 | 4144 | 2795 | 1942 | 4572 | 675 | 2927 | 2597 | 1322 | 1234 | 4231 |
| | 723 | 3713 | 3696 | 1398 | 4228 | 1569 | 863 | 1819 | 1667 | 1670 | 4249 | 3968 |
| | 552 | 923 | 303 | 305 | 1255 | 323 | 2389 | 1784 | | | | |
| 132: | 1842 | 1379 | 3525 | 3342 | 1535 | 1533 | 3618 | 1860 | 3989 | 3123 | 1520 | 1716 |
| | 2867 | 271 | 2627 | 3010 | 3747 | 1534 | 1530 | 682 | 423 | 3752 | 3443 | 3358 |
| | 4732 | 559 | 4350 | 505 | 877 | 3282 | 4196 | 1022 | 3081 | 4767 | 1284 | |
| 133: | 1628 | 480 | 3341 | 4415 | 1152 | 3236 | 2385 | 1074 | 2303 | 3429 | 3551 | 1161 |
| | 778 | 3578 | 4174 | 4731 | 4744 | 1592 | 636 | 402 | 4378 | 3139 | 3540 | 1659 |
| | 557 | 3371 | 2026 | 3709 | 1938 | 3523 | 664 | 3066 | 2197 | 4012 | 2851 | 1660 |
| | 3612 | 4180 | 3416 | 3230 | 1866 | 981 | 542 | 997 | 1091 | 2000 | 653 | 4289 |
| | 2870 | 826 | 1066 | | | | | | | | | |
| 134: | 4574 | 4745 | 2712 | 3439 | 3136 | 509 | 1387 | 1391 | 1390 | 1926 | 1754 | 3261 |
| | 3328 | 1607 | 1587 | 1595 | 1610 | 3995 | 3461 | 3701 | 1837 | 3403 | 3085 | 3898 |
| | 4567 | 2451 | 3297 | 1821 | 2414 | 4434 | 3873 | 1779 | 2486 | 1328 | 1420 | 1246 |
| | 1231 | 673 | 2261 | 3259 | 4532 | 2878 | 2164 | 1084 | 3765 | 474 | 2742 | |
| 135: | 4253 | 4257 | 2807 | 2808 | 2789 | 1799 | 1333 | 4636 | 1365 | 1361 | 4187 | 2234 |
| | 2825 | 815 | 811 | 1405 | 4245 | 3243 | 1518 | 2995 | 1506 | 2635 | 2640 | 2331 |
| | 2795 | 3633 | 2264 | 4573 | 723 | 2912 | 2111 | 2103 | 2662 | 4228 | 1667 | 1670 |
| | 1798 | 552 | 923 | 1255 | 323 | 2389 | 1784 | 4637 | 1809 | | | |
| 136: | 1250 | 2536 | 4347 | 3202 | 4419 | 1790 | 2293 | 2961 | 379 | 2616 | 1516 | 3561 |
| | 1577 | 764 | | | | | | | | | | |
| 137: | 4402 | 3160 | 3677 | 451 | 4395 | 2546 | 1402 | 1338 | 2398 | 4540 | 4622 | 4060 |
| | 3919 | 3008 | 3598 | 2989 | 2115 | 1808 | 3327 | 4501 | 479 | 858 | 3055 | 3718 |
| | 4804 | 2602 | 4413 | 386 | 2399 | 3001 | 4632 | 4493 | 2560 | 2161 | 373 | 1323 |
| | 1421 | 3430 | 428 | 3211 | 1060 | 3305 | 3311 | 2952 | 3458 | 336 | 3946 | 525 |
| | 3036 | 643 | 822 | | | | | | | | | |
| 138: | 268 | 3009 | | | | | | | | | | |
| 139. | 3889 | 4745 | 3136 | 1391 | 1926 | 1754 | 3261 | 3328 | 1610 | 1587 | 2322 | 3995 |
| | 893 | 3461 | 3701 | 2564 | 3197 | 2534 | 4510 | 3848 | 445 | 249 | 1131 | 2345 |
| | 4567 | 2451 | 3034 | 1052 | 660 | 2613 | 4799 | 713 | 1309 | 1501 | 2067 | 3873 |
| | 2486 | 2366 | 4715 | 4400 | 1195 | 3653 | 3259 | 343 | 367 | 2164 | 4392 | 1084 |
| | 686 | 474 | 3765 | | | | | | | | | |
| 140: | 1678 | 1233 | 3185 | 4682 | 3067 | 4362 | 1752 | 964 | 4339 | 656 | 4053 | 2654 |
| | 1257 | 4535 | 1298 | 4418 | 4547 | 2425 | 3493 | 1035 | 1770 | 2802 | 2387 | 453 |
| | 2964 | | | | | | | | | | | |
| 141: | 2542 | 1517 | 1865 | 4374 | 1881 | 3845 | 1987 | 2224 | 4080 | 1600 | 557 | 1588 |
| | 1207 | 962 | 4365 | 4169 | 1318 | 542 | 2109 | 997 | 1091 | 2000 | 653 | 2458 |
| | 3459 | 4648 | 3746 | | | | | | | | | |
| 142: | 2649 | 3341 | 1196 | 1800 | 1600 | 2446 | 2397 | 1207 | 962 | 4365 | 4140 | 4695 |
| | 542 | 1091 | 997 | 3300 | 930 | 3962 | 1273 | | | | | |
| 143: | 1973 | 2178 | 2823 | 815 | 811 | 247 | 1178 | 1518 | 2428 | 1930 | 1915 | 2795 |
| | 3604 | 2927 | 1495 | 1776 | 552 | 923 | 323 | 2389 | 914 | 4158 | 3414 | |
| 144: | 1143 | 3272 | 3274 | 3315 | 1854 | 245 | 1518 | 4528 | 2795 | 4383 | 1299 | 1277 |
| | 4338 | 2927 | 4267 | 4265 | 345 | 2723 | 3383 | 4571 | 2432 | 4758 | 534 | 1748 |
| | 3751 | 4132 | 3483 | 2900 | 1254 | 397 | 3343 | 2642 | 1667 | 1670 | 4249 | 2573 |
| | 4178 | 4363 | 595 | 552 | 923 | 792 | 2444 | 2389 | 2396 | 759 | 2740 | 463 |
| 145: | 1293 | 1263 | 1214 | 3885 | 4490 | 2579 | 1932 | 4577 | 4334 | 714 | 3682 | 280 |
| | 1416 | 786 | 3595 | 2619 | 2461 | 2540 | 3260 | 4199 | 796 | 4202 | 4090 | 528 |
| | 912 | 435 | 3338 | 632 | 1900 | 537 | 2598 | 288 | 2447 | 4047 | 2716 | 1367 |
| | 501 | 2151 | 2720 | 1564 | 3507 | 4299 | | | | | | |
| 146: | 4508 | 2457 | 2498 | 4463 | 1198 | 1202 | 1522 | 4137 | 168 | 1686 | 3401 | 2996 |
| | 2776 | 3224 | 2656 | 1927 | 744 | 795 | 4726 | 3222 | 3183 | 1434 | 4447 | 4500 |
| | 2015 | 2854 | 2839 | 2836 | 2852 | 3789 | 4157 | 4737 | 741 | 4270 | 1024 | 2180 |
| | 710 | 4342 | 4779 | 3584 | 3976 | 3529 | 1898 | 1270 | 2947 | 3075 | 3276 | 4016 |
| | 2473 | 2163 | 262 | 4564 | 4134 | 2330 | | | | | | |
| 147: | 2494 | 1573 | 1553 | 1591 | 1589 | 4051 | 2323 | 3221 | 1699 | 1702 | 2525 | 4319 |
| | 4139 | 4141 | 3017 | 3016 | 1680 | 2994 | 251 | 1969 | 4478 | 2416 | 4634 | 3825 |
| | 2478 | 2419 | 3377 | 867 | 1697 | 1718 | 1715 | 1677 | 1675 | 2460 | 2372 | 1840 |
| | 3771 | 4733 | 1694 | 1690 | 1712 | 4201 | 3076 | 2637 | 2634 | 794 | 784 | 791 |
| | 813 | 2604 | 2360 | 2529 | 3124 | 1113 | 1989 | 2463 | 2479 | 4304 | 3465 | 2978 |
| | 3799 | 4550 | 974 | 2301 | 520 | 1643 | 1671 | 1646 | 3932 | 4538 | 644 | 1846 |
| | 1901 | 4613 | 2971 | 4808 | 4298 | 4359 | 1321 | 2289 | 1316 | 2953 | 3356 | 1783 |
| | 3252 | 3472 | 3107 | 1751 | 910 | 1743 | 4711 | 2352 | 3914 | 2485 | 4127 | 1730 |
| | 2219 | 242 | 586 | 3080 | 3102 | 2813 | 3602 | 2792 | 2483 | 2559 | 2553 | 302 |
| | 282 | 3109 | 1641 | 536 | 4264 | 3473 | 2576 | | | | | |
| 148: | 4792 | 1080 | 1818 | 2039 | 1864 | 557 | 997 | 1091 | 2000 | 653 | 3444 | |
| 149: | 401 | 2840 | 324 | 2005 | 1347 | 2549 | 1519 | 2063 | 4421 | 3326 | 1256 | |
| 150: | 3320 | 634 | 1294 | 378 | 2057 | 592 | 617 | 614 | 2862 | 3817 | 267 | 1357 |
| | 1355 | 1354 | 1314 | 4013 | 2134 | 408 | 1994 | 3091 | 3629 | 3111 | 3593 | 2313 |
| | 4497 | 1484 | 2048 | 381 | 3451 | 1289 | 2516 | 1514 | 2569 | 2195 | 1525 | 1531 |
| | 1509 | 1523 | 2070 | 2948 | 2519 | 4544 | 4296 | 3996 | 4606 | 2448 | 2688 | 321 |
| | 3694 | 589 | 3670 | 1481 | 3902 | 363 | 2110 | 4006 | 4007 | 4008 | 3509 | 1666 |
| | 3070 | 3028 | 4759 | 1982 | 279 | 1562 | 3770 | 1540 | 3874 | 524 | 2296 | 2241 |
| | 4368 | 2210 | 4259 | 2129 | 1090 | 3830 | 2832 | 1123 | 3518 | 909 | 3745 | 620 |
| | 4593 | 3520 | 4694 | 635 | 618 | 3695 | | | | | | |
| 151: | 4477 | 640 | 213 | 3024 | 4491 | 3762 | 1929 | 4645 | 2609 | 4215 | 4484 | 355 |
| | 3499 | 3114 | 1088 | 1076 | 3433 | 1688 | 3238 | 3505 | 4724 | 2381 | 1304 | 297 |
| | 4273 | 506 | 4689 | 1448 | | | | | | | | |
| 152: | 3320 | 3756 | 3758 | 3699 | 2028 | 375 | 2988 | 4729 | 1986 | 267 | 1357 | 1354 |
| | 1355 | 1314 | 3178 | 4592 | 4591 | 3091 | 4485 | 3582 | 3111 | 3591 | 4346 | 4673 |
| | 2731 | 580 | 598 | 2672 | 1377 | 2967 | 3398 | 4794 | 3902 | 1940 | 3797 | 3558 |
| | 4423 | 1982 | 3270 | 3309 | 3645 | 4593 | 2572 | 511 | 4694 | 4438 | 2079 | 747 |
| | 3040 | 3983 | 568 | 1411 | 2206 | 3644 | | | | | | |
| 153: | 1460 | 3242 | 4430 | 1829 | 4141 | 1680 | 3764 | 4293 | 3941 | 1546 | 3825 | 3834 |
| | 3175 | 1697 | 1677 | 1675 | 4468 | 4266 | 1712 | 1694 | 1690 | 2871 | 4004 | 4654 |
| | 1370 | 4669 | 1984 | 1193 | 1172 | 1194 | 3076 | 2223 | 1073 | 4333 | 797 | 813 |
| | 2360 | 3799 | 4550 | 2728 | 3975 | 2301 | 1671 | 1643 | 3669 | 1578 | 1901 | 1096 |
| | 2276 | 897 | 436 | 3755 | 1713 | 3283 | 3018 | 4135 | 3779 | 2803 | 266 | 500 |
| | 910 | 2252 | 1743 | 753 | 3163 | 3162 | 1730 | 2219 | 3256 | 4183 | 3367 | 2689 |
| | 2765 | 3623 | 3437 | 1641 | 3610 | 3144 | 1887 | 4264 | 3805 | 2853 | 872 | 1463 |
| 154: | 349 | 1453 | 4088 | 1176 | 2379 | 2142 | 1331 | 946 | 3969 | 4707 | 4552 | 1841 |
| | 4153 | 2942 | 3482 | 263 | 3605 | 4515 | 2643 | 2298 | 2299 | 2513 | 1219 | 1319 |
| | 951 | 326 | 1549 | 4025 | 1026 | 1027 | 2799 | 3271 | 715 | 1793 | 1637 | 1515 |
| | 2246 | 4279 | 562 | | | | | | | | | |
| 155: | 2709 | 4268 | 1083 | 3548 | 4112 | 1810 | | | | | | |
| 156: | 3424 | 3399 | 2865 | 4036 | 2073 | 543 | 4505 | 359 | 2842 | 3462 | 2905 | 459 |
| | 2719 | 4616 | 3541 | 3985 | 1629 | 2626 | 783 | 638 | 3859 | 707 | 4035 | 1184 |
| 157: | 988 | 236 | 255 | 301 | 346 | 3905 | 963 | 1004 | 4687 | 296 | 1009 | 904 |
| | 901 | 879 | 3928 | 3929 | 1162 | 3927 | 3912 | 1046 | 2600 | 959 | 889 | 885 |
| | 4401 | 384 | 3292 | 1759 | 3454 | 4328 | 295 | 3382 | 2452 | 766 | 1863 | 2760 |
| | 3524 | 3503 | 3485 | 3506 | 3836 | 3714 | 4611 | 332 | 3556 | 3484 | 3477 | 3481 |
| | 3339 | 3062 | 3456 | 4710 | 1086 | 583 | 2701 | 2647 | 2167 | 2165 | 2704 | 2149 |
| | 2699 | 3352 | 3319 | 3440 | 3393 | 3333 | 3301 | 3336 | 3904 | 3954 | 4001 | 3982 |
| | 3979 | 3665 | 3664 | 3861 | 3858 | 3955 | 3953 | 3865 | 3977 | 3907 | 3909 | 3876 |
| | 3787 | 3447 | 3547 | 3544 | 3539 | 3527 | 2025 | 625 | 697 | 1188 | 4076 | 3069 |
| | 2306 | 2307 | 388 | 2361 | 234 | 400 | 1681 | 1583 | 1547 | 376 | 4226 | 2237 |
| | 2034 | 3302 | 3662 | 2071 | 2856 | 1078 | 1789 | 1107 | 3445 | 3879 | 3950 | 3951 |
| | 3903 | 3882 | 3930 | 3931 | 3862 | 3864 | 3959 | 3981 | 3978 | 3875 | 4725 | 4716 |
| | 1883 | 1378 | 2119 | 1888 | 1873 | 1871 | 4646 | 1869 | 986 | 4713 | 1816 | |
| 158: | 3729 | 2266 | 2722 | 1410 | 680 | 2644 | 932 | 3974 | 2135 | 1703 | 2232 | 891 |
| | 2242 | 2238 | 2265 | 1647 | 2190 | 2269 | 903 | 1158 | 3310 | 1536 | 541 | 1615 |
| | 4256 | | | | | | | | | | | |
| 159: | 988 | 255 | 301 | 346 | 3905 | 963 | 1004 | 4687 | 1009 | 901 | 879 | 1162 |
| | 3927 | 1145 | 4145 | 1835 | 2320 | 2600 | 959 | 889 | 885 | 4401 | 384 | 2523 |
| | 4332 | 4335 | 3396 | 4318 | 1924 | 2517 | 3456 | 4710 | 1086 | 3577 | 2408 | 3858 |
| | 3861 | 3955 | 3907 | 3909 | 3876 | 3447 | 3547 | 3544 | 3539 | 3527 | 2431 | 388 |
| | 2418 | 1691 | 4473 | 3445 | 3879 | 3951 | 3950 | 3864 | 3862 | 3959 | 3981 | 3978 |
| | 3875 | 4716 | 4725 | 1883 | 986 | 4765 | | | | | | |
| 160: | 2166 | 3361 | 2042 | 4437 | 4450 | 4706 | 712 | 4777 | 4233 | 2089 | 1581 | |
| 161: | 988 | 236 | 255 | 301 | 963 | 1004 | 4687 | 296 | 1009 | 904 | 901 | 879 |
| | 3928 | 3929 | 3927 | 4145 | 1046 | 2600 | 959 | 889 | 885 | 4401 | 384 | 3357 |
| | 2201 | 2281 | 2949 | 717 | 261 | 4668 | 491 | 3267 | 4382 | 3456 | 4710 | 1086 |
| | 2408 | 3319 | 3333 | 3440 | 3393 | 3301 | 3352 | 3336 | 3954 | 3904 | 3447 | 3547 |
| | 3544 | 3539 | 3527 | 1744 | 4287 | 388 | 3516 | 3354 | 2386 | 3452 | 3683 | 1078 |
| | 1789 | 1107 | 3445 | 4716 | 4725 | 1883 | 1378 | 2119 | 986 | 1129 | | |
| 162: | 1387 | 2858 | 749 | 4717 | 3820 | 746 | 3837 | 857 | 3793 | 3657 | 2080 | 2414 |
| | 1821 | 4434 | 3855 | 294 | 2105 | 4059 | 886 | 1217 | 864 | 2037 | 4532 | 2742 |
| | 985 | 4772 | | | | | | | | | | |
| 163: | 760 | 4005 | 3088 | 2102 | 1435 | 2243 | 2847 | 2845 | 2587 | 1899 | 2262 | |
| 164: | 988 | 4352 | 236 | 255 | 301 | 346 | 3905 | 963 | 1004 | 4687 | 296 | 1009 |
| | 904 | 901 | 879 | 3928 | 3929 | 1162 | 3927 | 1046 | 845 | 959 | 889 | 885 |
| | 816 | 1075 | 4151 | 584 | 606 | 560 | 523 | 535 | 504 | 1653 | 3382 | 766 |
| | 1875 | 3323 | 3456 | 4710 | 1086 | 583 | 3319 | 3333 | 3393 | 3301 | 3954 | 3352 |
| | 3336 | 3440 | 3904 | 3861 | 3858 | 3955 | 3865 | 3977 | 3907 | 3909 | 3876 | 3787 |
| | 3447 | 3544 | 3547 | 3539 | 3527 | 4272 | 2306 | 2307 | 388 | 2361 | 977 | 1078 |
| | 1789 | 1107 | 3445 | 3879 | 3951 | 3950 | 3882 | 3903 | 3930 | 3931 | 3862 | 3864 |
| | 3959 | 3981 | 3978 | 3875 | 4716 | 4725 | 1883 | 1378 | 2119 | 986 | | |
| 165: | 1294 | 375 | 412 | 413 | 267 | 2562 | 4013 | 4485 | 3591 | 4673 | 4346 | 580 |
| | 598 | 4488 | 4575 | 1502 | 2198 | 2652 | 2849 | 1069 | 3058 | 3902 | 3753 | 1940 |
| | 3269 | 393 | 3509 | 3410 | 933 | 4087 | 1302 | 3774 | 1982 | 342 | 337 | 340 |
| | 319 | 4368 | 663 | 684 | 3015 | 2783 | 3168 | 4694 | 357 | 4456 | 484 | 2475 |
| | 1419 | 1807 | | | | | | | | | | |
| 166: | 1363 | 4115 | 2693 | 2690 | 443 | 375 | 971 | 2343 | 2346 | 2351 | 743 | 4013 |
| | 2056 | 2074 | 3318 | 690 | 3565 | 2547 | 553 | 4061 | 4063 | 2112 | 915 | 3781 |
| | 2371 | 2902 | 2945 | 2901 | 1903 | 2584 | 4308 | 3190 | 4559 | 4446 | 648 | 2499 |
| | 1921 | 1528 | 958 | 473 | 444 | 446 | 470 | 468 | 465 | 957 | 4010 | 841 |
| | 395 | 843 | 960 | 1396 | 3122 | 3724 | 2449 | 2876 | 3509 | 1967 | 253 | 4445 |
| | 4558 | 4368 | 3702 | 1951 | 1464 | 4674 | 4595 | 4118 | 2349 | 3520 | 619 | 3521 |
| | 2354 | 511 | 742 | 1735 | 4143 | 2100 | | | | | | |
| 167: | 1325 | 1725 | 2821 | 2903 | 494 | 1830 | 3501 | 4690 | 2152 | 4635 | 3703 | 2500 |
| | 3304 | 438 | 1496 | 4381 | 4667 | 4579 | 4389 | 4397 | 4312 | 419 | 2255 | 4514 |
| | 4741 | 2899 | 3988 | 441 | 945 | 1395 | 2714 | 2749 | 2024 | 873 | 668 | 1912 |
| | 3078 | 4386 | 4323 | 3918 | 2001 | 3748 | 3600 | 3737 | 1768 | 2745 | 3316 | 1417 |
| | 1868 | 2707 | 4030 | 3790 | 649 | 772 | | | | | | |
| 168: | 3730 | 4508 | 2457 | 2498 | 1438 | 718 | 3743 | 2857 | 146 | 2330 | 4163 | 582 |
| | 4354 | 2209 | 1950 | 629 | 3475 | 2049 | 3027 | 4463 | 2501 | 1997 | 3530 | 3234 |
| | 2017 | 1198 | 1202 | 1927 | 4726 | 795 | 3298 | 748 | 3222 | 978 | 3576 | 4789 |
| | 1456 | 2412 | 1253 | 3183 | 1434 | 4447 | 2015 | 2854 | 3789 | 4157 | 4737 | 790 |
| | 3754 | 1719 | 1216 | 1627 | 1111 | 1024 | 741 | 2180 | 4270 | 3584 | 710 | 4779 |
| | 4342 | 3976 | 3529 | 1898 | 3075 | 1270 | 2947 | 3276 | 262 | 2217 | 4016 | 2163 |
| | 4564 | 2473 | 4134 | 4137 | 1522 | | | | | | | |
| 169: | 2963 | 1567 | 3731 | 2383 | 572 | 1266 | 3698 | 3280 | 2318 | 4454 | 563 | 1308 |
| | 4633 | 1714 | 1920 | 1011 | 2696 | 1524 | 4678 | 2411 | 3783 | 1910 | 271 | 3010 |
| | 1045 | 836 | 4666 | 4186 | 1432 | 2130 | 3317 | 3872 | 3432 | 682 | 2183 | 3822 |
| | 4409 | 812 | 4301 | 433 | 3634 | 1454 | | | | | | |
| 170: | 3688 | 3255 | 3188 | 3167 | 3073 | 3072 | 3690 | 3235 | 3150 | 3097 | 3094 | 3092 |
| | 3090 | 3715 | 3200 | 2168 | 2032 | 3594 | 1527 | 3174 | 2325 | 1372 | 993 | 390 |
| | 3106 | 3400 | 825 | 3588 | 3984 | 4252 | 3636 | 1630 | 2308 | 3431 | 3161 | 4045 |
| | 4701 | 411 | 1386 | 1855 | 2538 | 1249 | 1428 | 399 | 1210 | | | |
| 171: | 1998 | 4106 | 3065 | 488 | 1753 | 2054 | 3533 | 4114 | 4219 | 2916 | 4077 | 2259 |
| | 621 | 1962 | 929 | 4728 | 2083 | 391 | 2521 | 3606 | 4357 | 1003 | 1498 | 3345 |
| | 3022 | 1565 | | | | | | | | | | |
| 172: | 1403 | 1548 | 1545 | 2273 | 2963 | 1379 | 920 | 4324 | 2897 | 3086 | 3568 | 530 |
| | 4633 | 1535 | 1533 | 3967 | 2128 | 1206 | 1537 | 1952 | 2753 | 733 | 4171 | 271 |
| | 3054 | 3030 | 2907 | 3010 | 3546 | 1276 | 1833 | 4186 | 1432 | 1240 | 674 | 2518 |
| | 1148 | 2208 | 456 | 2076 | 1616 | 3538 | 4396 | 2155 | 3324 | 982 | 1534 | 1530 |
| | 4516 | 1633 | 344 | 2072 | 3137 | 2997 | 565 | 2524 | 2991 | 235 | 1941 | 2125 |
| | 4403 | 4167 | 3450 | 2541 | 1722 | 4177 | 2834 | 3013 | 1794 | 2700 | 2333 | 4426 |
| | 3358 | 3443 | 613 | 631 | 615 | 4732 | 3634 | 709 | 3127 | | | |
| 173: | 878 | 3586 | 2171 | 3138 | 3794 | 1345 | 3844 | 4699 | 2279 | 4015 | 4801 | 775 |
| | 2641 | 3615 | 2895 | 4705 | 1884 | 1422 | 987 | | | | | |
| 174: | 2497 | 1118 | 1460 | 1459 | 3463 | 3412 | 4569 | 2981 | 2681 | 3221 | 2377 | 4225 |
| | 2525 | 2515 | 1829 | 4191 | 3390 | 3387 | 3763 | 2994 | 251 | 1479 | 1975 | 4478 |
| | 2416 | 4634 | 1544 | 2476 | 2478 | 2424 | 3376 | 867 | 3175 | 1503 | 2460 | 489 |
| | 490 | 2372 | 2392 | 3047 | 1608 | 4733 | 1747 | 2871 | 4004 | 3560 | 1965 | 3460 |
| | 4229 | 4227 | 3044 | 2634 | 2192 | 1073 | 4333 | 3832 | 3064 | 3061 | 784 | 791 |
| | 3039 | 2604 | 2360 | 1890 | 2529 | 2550 | 3124 | 1113 | 3491 | 462 | 4685 | 1136 |
| | 1989 | 1013 | 3465 | 2978 | 2979 | 3799 | 518 | 806 | 3370 | 3116 | 3975 | 4785 |
| | 4783 | 4722 | 4385 | 1836 | 2216 | 2218 | 2193 | 705 | 2191 | 3308 | 3871 | 3961 |
| | 644 | 3490 | 3453 | 4203 | 4208 | 3471 | 2691 | 3727 | 2312 | 472 | 2926 | 2508 |
| | 1317 | 2280 | 711 | 859 | 2570 | 4723 | 1316 | 4150 | 3394 | 1783 | 2023 | 2035 |
| | 2465 | 2467 | 2468 | 1164 | 1167 | 3472 | 3348 | 3467 | 3018 | 3474 | 3107 | 3492 |
| | 4384 | 4211 | 3496 | 3497 | 1778 | 4205 | 3922 | 1886 | 4417 | 2249 | 496 | 2254 |
| | 700 | 3914 | 2352 | 3163 | 2485 | 2484 | 1362 | 4786 | 4780 | 2674 | 4784 | 882 |
| | 3923 | 3925 | 241 | 1559 | 586 | 2043 | 2138 | 1593 | 2812 | 1429 | 1376 | 233 |
| | 4815 | 2483 | 2482 | 3437 | 2559 | 2566 | 1992 | 2586 | 2555 | 2631 | 2614 | 302 |
| | 282 | 3045 | 1831 | 2438 | 1785 | 3148 | 3142 | 3473 | 2337 | 2275 | | |
| 175: | 871 | 3176 | 4274 | 773 | 1452 | 1271 | 4482 | 1977 | 4390 | 4526 | 4521 | 1272 |
| | 1604 | 2423 | 4026 | 3037 | 276 | 1905 | 3042 | 853 | 823 | 4058 | 510 | 3880 |
| 176: | 3424 | 1334 | 2204 | 4055 | 2472 | 1597 | 800 | 4431 | 1229 | 4358 | 4607 | 3908 |
| | 3511 | 3648 | 2169 | 1315 | 906 | 4523 | 4465 | 4471 | 2286 | 848 | 4017 | 638 |
| | 3859 | 3172 | | | | | | | | | | |
| 177: | 1682 | 3778 | 3772 | 1185 | 3792 | 3719 | 1048 | 1051 | 1056 | 1059 | 1054 | 3738 |
| | 4160 | 1795 | 3788 | 3791 | 4568 | 4664 | 3628 | 3006 | 2982 | 1082 | 1085 | 1014 |
| | 2679 | 3722 | 3215 | 3217 | 3216 | 999 | 300 | 304 | 4009 | 1393 | 728 | 2582 |
| | 4192 | 2725 | 2755 | 2754 | 3103 | 1829 | 3019 | 4812 | 4325 | 4326 | 1972 | 1125 |
| | 4479 | 689 | 2622 | 1433 | 4064 | 2777 | 2775 | 3941 | 1546 | 3313 | 3194 | 2127 |
| | 4355 | 1000 | 4773 | 3627 | 2405 | 2880 | 4753 | 3671 | 2101 | 3366 | 793 | 3374 |
| | 2846 | 4657 | 3728 | 3725 | 2460 | 4082 | 2744 | 2741 | 2773 | 2758 | 2770 | 2757 |
| | 2772 | 3575 | 973 | 968 | 966 | 996 | 1106 | 1104 | 1103 | 1110 | 2893 | 2909 |
| | 2937 | 1983 | 1981 | 3991 | 1683 | 2253 | 2344 | 4782 | 4760 | 4756 | 4754 | 3560 |
| | 1965 | 605 | 609 | 3135 | 3806 | 2756 | 3128 | 1020 | 1031 | 1030 | 1019 | 4442 |
| | 4441 | 2891 | 2655 | 3720 | 3717 | 2215 | 2743 | 905 | 908 | 2186 | 2202 | 2088 |
| | 455 | 1978 | 1979 | 2078 | 1114 | 4685 | 1510 | 4548 | 2301 | 1668 | 835 | 849 |
| | 838 | 529 | 1870 | 4432 | 3915 | 4216 | 2605 | 299 | 2892 | 1204 | 1201 | 4749 |
| | 4380 | 1166 | 284 | 995 | 2939 | 2910 | 1227 | 1221 | 1205 | 3784 | 404 | 1468 |
| | 1265 | 4002 | 2686 | 3960 | 1781 | 1780 | 4049 | 1171 | 1186 | 3526 | 3392 | 3549 |
| | 3987 | 4031 | 4052 | 4065 | 3099 | 1225 | 3281 | 1676 | 1622 | 1580 | 2228 | 3478 |
| | 1679 | 2010 | 1695 | 1701 | 1674 | 3257 | 4416 | 4649 | 2739 | 2736 | 4677 | 4665 |
| | 694 | 1976 | 1959 | 1777 | 1343 | 1954 | 1937 | 1751 | 4084 | 3952 | 4294 | 4810 |
| | 4037 | 4811 | 4250 | 950 | 2879 | 869 | 1693 | 3936 | 3935 | 3934 | 1288 | 590 |
| | 4100 | 2748 | 2833 | 1425 | 2310 | 4097 | 3708 | 1934 | 1344 | 1342 | 1385 | 4127 |
| | 3920 | 4680 | 4681 | 550 | 722 | 1943 | 1945 | 3651 | 1787 | 1197 | 3901 | 2665 |
| | 230 | 387 | 2663 | 2661 | 2657 | 3129 | 2091 | 3100 | 1658 | 1631 | 1582 | 1623 |
| | 1624 | 1585 | 1640 | 1598 | 1642 | 1599 | 1601 | 1645 | 1603 | 2285 | 1648 | 1605 |
| | 1652 | 3173 | 4095 | 913 | 928 | 927 | 924 | 3115 | 2581 | 495 | 2094 | 2917 |
| | 2896 | 1760 | 4594 | 1928 | 1925 | 1956 | 1957 | 1955 | 3739 | 4264 | 274 | 3899 |
| 178: | 2509 | 4461 | 1415 | 3486 | 3277 | 3545 | 3002 | 2292 | 512 | 2822 | 3619 | 4138 |
| | 1079 | 3896 | 4803 | 3635 | 2898 | 954 | 1212 | 4487 | 1310 | 1154 | 2544 | 1731 |
| | 2329 | 2771 | 4807 | 3883 | 4679 | 351 | 3510 | 4379 | 4533 | 3890 | 1834 | 2552 |
| | 513 | 573 | 1738 | 1109 | 831 | 2620 | 4398 | 881 | 4204 | 2561 | 1826 | 4050 |
| | 2012 | 1443 | 3079 | 1003 | 2113 | | | | | | | |
| 179: | 4608 | 4453 | 2712 | 3439 | 3136 | 3295 | 4212 | 1406 | 1409 | 1371 | 1404 | 1754 |
| | 3261 | 1587 | 1610 | 1612 | 1607 | 1595 | 2322 | 3995 | 3219 | 4405 | 3096 | 1450 |
| | 3461 | 3701 | 4091 | 3824 | 1213 | 1897 | 2615 | 1174 | 4567 | 2451 | 1478 | 3033 |
| | 3808 | 1427 | 2887 | 2884 | 2889 | 2924 | 3813 | 2095 | 1307 | 2067 | 2154 | 3259 |
| | 4532 | 2878 | 2164 | 4392 | 1084 | 2677 | 474 | 3765 | 2493 | 2539 | 2973 | |
| 180: | 2494 | 2981 | 2373 | 2376 | 3221 | 1699 | 1702 | 4460 | 2525 | 4430 | 1829 | 251 |
| | 2994 | 2416 | 3941 | 1546 | 3825 | 2478 | 2419 | 2424 | 3101 | 3376 | 2843 | 2844 |
| | 3175 | 1697 | 1718 | 1715 | 1677 | 1675 | 1503 | 2460 | 4468 | 2441 | 3771 | 1840 |
| | 4322 | 4733 | 1690 | 1712 | 1694 | 4184 | 3603 | 4004 | 4654 | 1370 | 4669 | 4661 |
| | 3021 | 1193 | 1172 | 1194 | 1041 | 1037 | 1040 | 1039 | 4172 | 4280 | 1102 | 4201 |
| | 3044 | 3076 | 2637 | 2223 | 797 | 813 | 784 | 2604 | 2360 | 2529 | 3124 | 4685 |
| | 1139 | 2886 | 1989 | 1909 | 3799 | 4550 | 3975 | 4797 | 704 | 1671 | 1643 | 1646 |
| | 1661 | 2188 | 1247 | 644 | 3490 | 3453 | 3471 | 4208 | 4462 | 3550 | 4298 | 676 |
| | 315 | 1944 | 4723 | 1316 | 4277 | 1783 | 1882 | 1190 | 3472 | 4524 | 3467 | 3018 |
| | 3107 | 3492 | 4211 | 3496 | 3497 | 460 | 2176 | 1360 | 458 | 701 | 440 | 4223 |
| | 854 | 3947 | 3809 | 4766 | 4043 | 1758 | 910 | 1743 | 2957 | 3209 | 3208 | 604 |
| | 3231 | 2159 | 753 | 3163 | 2485 | 3162 | 2733 | 884 | 1730 | 3256 | 4183 | 2765 |
| | 3602 | 1948 | 1466 | 2559 | 1121 | 1773 | 2553 | 1634 | 2438 | 1742 | 1737 | 1641 |
| | 3610 | 1887 | 3473 | 1827 | 1970 | 1359 | 655 | 293 | | | | |
| 181: | 2781 | 461 | 3916 | 2746 | 803 | 362 | 3956 | 2309 | 4719 | | | |
| 182: | 4106 | 2132 | 1669 | 193 | 3196 | 1512 | 1497 | 1050 | 2766 | 1498 | 1099 | 4218 |
| | 671 | 4108 | 2297 | | | | | | | | | |
| 183: | 2503 | 1500 | 2116 | 2671 | 1467 | 2214 | 277 | 1700 | 3052 | 1550 | 824 | 1880 |
| 184: | 2883 | 3007 | 3827 | 4079 | 2045 | 4104 | 2137 | 328 | 1408 | 2327 | 1157 | 1071 |
| | 3744 | 3504 | 4029 | 4027 | 4457 | 2721 | 2406 | 729 | 1126 | 1874 | 330 | 862 |
| | 290 | 2955 | 4113 | 2020 | 1116 | 1839 | | | | | | |
| 185: | 3385 | 2090 | 4000 | 1542 | 2687 | 2693 | 2690 | 2028 | 443 | 375 | 378 | 412 |
| | 413 | 352 | 1337 | 2562 | 3566 | 610 | 1098 | 4343 | 2454 | 3611 | 3608 | 949 |
| | 4375 | 4517 | 3658 | 2678 | 1947 | 482 | 4446 | 2796 | 2311 | 2362 | 2300 | 3797 |
| | 4542 | 957 | 444 | 446 | 958 | 473 | 470 | 468 | 465 | 4010 | 3159 | 393 |
| | 2357 | 2402 | 1895 | 389 | 2145 | 4410 | 4407 | 4443 | 4425 | 3640 | 2830 | 2008 |
| | 4445 | 342 | 337 | 340 | 3512 | 967 | 1704 | 2875 | 1380 | 3438 | 2751 | 856 |
| | 4448 | 4599 | 2837 | 2783 | 3520 | 4372 | 4360 | 4190 | 2673 | 2658 | 511 | 1584 |
| | 3108 | 2762 | 1745 | 3112 | | | | | | | | |
| 186: | 2132 | 1532 | 3533 | 4219 | 4114 | 2916 | 4077 | 2046 | 3878 | 2510 | 1146 | 1072 |
| | 1003 | 1498 | 1099 | | | | | | | | | |
| 187: | 975 | 972 | 3532 | 3513 | 4042 | 1475 | 1239 | 532 | 4704 | 1261 | 2522 | 2287 |
| | 1656 | 3831 | 1286 | 327 | 447 | 911 | 318 | 317 | 3050 | 3590 | 2036 | 3332 |
| | 687 | 801 | 1042 | 679 | 616 | 3378 | 2006 | 487 | 256 | | | |
| 188: | 3320 | 3758 | 3756 | 4000 | 2693 | 2687 | 2690 | 4718 | 375 | 378 | 2245 | 2248 |
| | 1097 | 608 | 2562 | 610 | 1098 | 3178 | 2134 | 339 | 894 | 1490 | 2454 | 4034 |
| | 3607 | 4439 | 4198 | 4083 | 4518 | 2826 | 1949 | 699 | 4446 | 3753 | 3159 | 393 |
| | 2357 | 2402 | 1895 | 3410 | 2145 | 4410 | 4407 | 4443 | 4425 | 2150 | 3893 | 4445 |
| | 1285 | 4674 | 612 | 1101 | 4599 | 2837 | 2783 | 4190 | 2673 | 2658 | 278 | 3105 |
| 189: | 2494 | 3158 | 3625 | 1460 | 4569 | 2981 | 4051 | 2373 | 1699 | 1702 | 2395 | 2525 |
| | 4430 | 1829 | 3035 | 3023 | 1680 | 3764 | 2356 | 4483 | 2416 | 2415 | 4634 | 3941 |
| | 3825 | 2478 | 1392 | 2419 | 2421 | 2436 | 2424 | 2394 | 2843 | 2844 | 3175 | 1718 |
| | 1697 | 1715 | 1677 | 1675 | 4643 | 4570 | 1503 | 4468 | 2378 | 2441 | 1840 | 3771 |
| | 1712 | 1690 | 1694 | 1766 | 2871 | 4184 | 3603 | 4654 | 1370 | 4004 | 4669 | 3560 |
| | 2064 | 4660 | 3003 | 1193 | 1172 | 1194 | 2645 | 4072 | 3220 | 3076 | 2223 | 797 |
| | 813 | 2604 | 1163 | 2866 | 3491 | 252 | 250 | 1139 | 2886 | 1909 | 306 | 1486 |
| | 2463 | 2479 | 3641 | 3799 | 4550 | 2599 | 3975 | 2301 | 4797 | 704 | 1671 | 1643 |
| | 1646 | 3669 | 2196 | 1247 | 644 | 1373 | 3490 | 3453 | 4203 | 4208 | 3471 | 2162 |
| | 2683 | 4162 | 4778 | 4723 | 4277 | 2953 | 1783 | 3225 | 2059 | 1170 | 3472 | 3264 |
| | 3263 | 1507 | 3467 | 3474 | 3492 | 4384 | 3496 | 4211 | 3497 | 3237 | 910 | 1743 |
| | 3957 | 3939 | 3914 | 2352 | 2350 | 2957 | 3208 | 753 | 1349 | 1382 | 2578 | 1730 |
| | 4183 | 1555 | 1556 | 3367 | 2765 | 1593 | 1376 | 1651 | 3602 | 1948 | 3437 | 2559 |
| | 1121 | 2555 | 2553 | 302 | 282 | 1636 | 3239 | 3117 | 2438 | 2440 | 1734 | 1765 |
| | 1641 | 3610 | 1673 | 4264 | 3473 | | | | | | | |
| 190: | 2494 | 3625 | 4236 | 4237 | 3412 | 2179 | 4569 | 2981 | 4051 | 3221 | 1699 | 1702 |
| | 2395 | 3241 | 4200 | 2525 | 4430 | 4141 | 4139 | 3035 | 1680 | 2356 | 4483 | 2416 |
| | 2415 | 4634 | 1392 | 2419 | 2421 | 2436 | 2424 | 2394 | 3175 | 1697 | 1718 | 1715 |
| | 1677 | 1675 | 4643 | 4570 | 1503 | 2460 | 2378 | 2392 | 2390 | 2443 | 2441 | 1690 |
| | 1694 | 1712 | 4736 | 2871 | 3603 | 4654 | 1370 | 2064 | 4660 | 1965 | 1984 | 3003 |
| | 1193 | 1172 | 1194 | 2645 | 4072 | 3220 | 3076 | 2220 | 813 | 767 | 769 | 3381 |
| | 2604 | 2360 | 2347 | 3491 | 252 | 2890 | 1579 | 3641 | 4304 | 3799 | 2732 | 4625 |
| | 4275 | 2763 | 546 | 1280 | 385 | 1904 | 3334 | 4550 | 4688 | 4133 | 3170 | 2301 |
| | 4797 | 704 | 1643 | 1671 | 1646 | 3669 | 2894 | 2496 | 1985 | 3958 | 3961 | 644 |
| | 3490 | 3453 | 4203 | 4208 | 3471 | 1901 | 1237 | 2575 | 2312 | 4708 | 3613 | 3949 |
| | 2636 | 2694 | 1292 | 2288 | 2289 | 2683 | 3761 | 4723 | 4150 | 4277 | 3394 | 1783 |
| | 2059 | 3252 | 3472 | 4524 | 3467 | 3018 | 3474 | 3107 | 1859 | 3537 | 976 | 2375 |
| | 3649 | 3359 | 799 | 4502 | 1203 | 3564 | 4601 | 3492 | 4384 | 3496 | 4211 | 3497 |
| | 4805 | 2278 | 650 | 4436 | 1094 | 3849 | 3642 | 312 | 1906 | 2992 | 457 | 4019 |
| | 3408 | 2009 | 348 | 3237 | 2118 | 1764 | 4170 | 1062 | 3542 | 1447 | 4530 | 2156 |
| | 2268 | 3926 | 1762 | 1141 | 3156 | 3379 | 2815 | 1451 | 4206 | 4166 | 3957 | 3939 |
| | 3914 | 2352 | 2350 | 2957 | 753 | 2485 | 1382 | 2578 | 1730 | 2219 | 4183 | 720 |
| | 1555 | 1556 | 3080 | 3102 | 2765 | 2689 | 1376 | 1117 | 1268 | 3602 | 4815 | 1948 |
| | 233 | 1466 | 3437 | 2559 | 1121 | 1773 | 2553 | 2555 | 2551 | 302 | 282 | 1636 |
| | 3239 | 3117 | 2438 | 2440 | 1734 | 1641 | 2778 | 3144 | 1303 | 1673 | 1887 | 4264 |
| | 1301 | 1327 | 3473 | 3418 | 1572 | | | | | | | |
| 191: | 3422 | 1173 | 2271 | 4411 | 3077 | 861 | 1933 | 4330 | 4769 | 3126 | 239 | 809 |
| | 3314 | 4290 | 3508 | 2703 | 3668 | 3291 | 3716 | 561 | 1538 | 4369 | 1857 | 3189 |
| | 2545 | 3273 | 637 | 2969 | 3973 | 3046 | 1055 | 602 | 3210 | 3212 | 1961 | 2507 |
| | 2505 | 3397 | 2667 | 1663 | 4345 | 3193 | 492 | 2669 | 571 | 984 | 1689 | 1705 |
| | 818 | 3678 | 3965 | 1775 | 3667 | 2365 | 4404 | 3278 | 1245 | 666 | | |
| 192: | 2818 | 2321 | 3500 | 2831 | 2750 | 2794 | 2793 | 309 | 4735 | 307 | 990 | 821 |
| | 2087 | 406 | 3785 | 4011 | 4046 | 2450 | 1353 | 3632 | 1655 | 1824 | 4393 | 4696 |
| | 1383 | 2404 | 372 | 3146 | 4329 | 1872 | 1449 | 1441 | 1444 | 1442 | 2420 | 3427 |
| | 953 | 956 | 1878 | 2593 | 3184 | 1440 | 2970 | 1296 | 2108 | 410 | | |
| 193: | 4106 | 2509 | 434 | 426 | 2259 | 1563 | 1561 | 2066 | 2041 | 4320 | 1187 | 1497 |
| | 2511 | 2510 | 3878 | 1458 | 1003 | 1498 | 1099 | 1512 | 1669 | | | |
| 194: | 2471 | 2520 | 3166 | 2251 | 2426 | 2558 | 4762 | 4085 | 448 | 1488 | 2983 | 3769 |
| | 2986 | 3767 | 4175 | 3937 | 4173 | 4675 | 2984 | 3815 | 4185 | 3811 | 2213 | 2085 |
| 195: | 1848 | 3647 | 860 | 3534 | 499 | 3721 | 789 | 1892 | 3306 | 2985 | 1461 | 2786 |
| | 3275 | 3468 | 3087 | 437 | 3800 | 2030 | 2477 | 2873 | 377 | 4507 | 2648 | 3692 |
| | 2930 | 2341 | 3321 | 3335 | 2231 | 4373 | 4376 | 350 | 4556 | 4763 | 4188 | 4496 |
| | 4281 | 322 | 4103 | 4742 | 4476 | 3782 | 1935 | 2624 | 1650 | 623 | 4412 | 1341 |
| | 2820 | 3464 | 4136 | 471 | 4721 | 1260 | 1557 | 3589 | 3051 | 2923 | 4207 | 870 |
| | 4408 | 1664 | 383 | 1053 | 1332 | 578 | 1381 | 1792 | 1736 | 2621 | 577 | 3228 |
| | 3804 | 2007 | 4336 | 3005 | 1575 | 4387 | | | | | | |
| 196: | 4129 | 1692 | 3621 | 2205 | 4429 | 3145 | 2353 | 4159 | 3290 | 1431 | 1437 | 3833 |
| | 3180 | 2768 | 1034 | 3656 | 2277 | 522 | 3213 | 2324 | 2060 | 1891 | 837 | 808 |
| | 517 | 4089 | 2968 | 1877 | 3402 | 4697 | 2437 | 3041 | 4310 | 3265 | 3802 | 3816 |
| | 2764 | 4306 | 1211 | 834 | 1369 | 1757 | 2580 | 2817 | 907 | 3118 | 2456 | 1100 |
| | 1252 | 2711 | 3661 | 2427 | 260 | 2121 | 2774 | 1375 | 403 | | | |
| 197: | 2888 | 2340 | 2730 | 1911 | 2869 | 1817 | 3839 | 4033 | 1267 | 659 | 3856 | 264 |
| | 1262 | 4529 | 287 | 936 | 1366 | 1008 | 2824 | 3130 | 3428 | 493 | 1057 | 3673 |
| | 2315 | 798 | 3689 | 4768 | | | | | | | | |
| 198: | 1015 | 3355 | 4300 | 275 | 4344 | 2120 | 2676 | 2284 | 2990 | 641 | 3143 | 2052 |
| | 3842 | 4119 | 2014 | 370 | 4662 | 4096 | 4152 | 1723 | 432 | 414 | 1436 | 3631 |
| | 3251 | 3963 | 3798 | 2422 | 4671 | 2999 | 1893 | 2738 | 2800 | 3853 | 2158 | 1149 |
| | 2585 | 2718 | 654 | 3363 | 3353 | 2780 | 2612 | 850 | 3933 | | | |
| 199: | 3795 | 4165 | 314 | 4506 | 382 | 3620 | 4560 | 3147 | 4739 | 3580 | 4596 | 1324 |
| | 4451 | 1815 | 1348 | 1346 | 661 | 1763 | 2710 | 651 | 1446 | 2175 | 4247 | 3466 |
| | 588 | 3515 | 1504 | 364 | 581 | 1822 | 1769 | 4295 | 4615 | 3442 | 409 | 1825 |
| | 2263 | 2194 | 1018 | 254 | 3293 | 1696 | 1771 | 2124 | 4509 | 1093 | 2504 | 4056 |
| | 4553 | 3583 | 3630 | 1224 | 2400 | 439 | | | | | | |
| 200: | 3900 | 2528 | 2316 | 1804 | 3488 | 1717 | 1953 | 3425 | 737 | 1064 | 1750 | 449 |
| | 3487 | 776 | 1179 | 2272 | 622 | 4269 | 2697 | 2029 | 4038 | 4504 | 392 | 1971 |
| | 427 | 3773 | 2018 | 2022 | 2031 | 2016 | 2021 | 691 | 1070 | 527 | 2737 | 3514 |
| | 3819 | 4422 | 2548 | 3659 | 1644 | 4311 | 844 | 1226 | 1389 | 1838 | | |
| 201: | 755 | 2004 | 3543 | 4356 | 752 | 3384 | 2274 | 2153 | 1609 | 464 | 4455 | 3587 |
| | 1964 | 3004 | 4494 | 3994 | 3614 | 4307 | 2114 | 4078 | 1228 | 1242 | 1554 | 1492 |
| | 2382 | 3705 | 2429 | 1772 | 1999 | 1791 | 4291 | 2235 | 2434 | 2606 | 2962 | |
| 202: | 2883 | 298 | 4590 | 852 | 1038 | 3786 | 3828 | 2267 | 2670 | 2933 | 2491 | 1071 |
| | 3744 | 2759 | 874 | 3043 | 1017 | 2998 | 4029 | 4027 | 4457 | 4761 | 1306 | 3186 |
| | 481 | 2596 | 3312 | 1708 | 1274 | 1092 | 916 | 3011 | 290 | 1487 | 2236 | |
| 203: | 3346 | 4602 | 2257 | 3863 | 2623 | 2827 | 1399 | 3528 | 4503 | 944 | 498 | 3676 |
| | 4800 | 817 | 1159 | 1049 | 3407 | 2430 | 3646 | 1511 | 980 | 1368 | 567 | 3406 |
| | 1412 | | | | | | | | | | | |
| 204: | 1403 | 2093 | 2273 | 1326 | 1483 | 2908 | 3596 | 3342 | 4633 | 1520 | 1716 | 750 |
| | 2492 | 2336 | 733 | 1222 | 4605 | 4171 | 526 | 3010 | 1602 | 1706 | 2931 | 4186 |
| | 1432 | 3686 | 1120 | 731 | 2140 | 734 | 1574 | 3712 | 768 | 4168 | 2187 | 3634 |
| | 2211 | | | | | | | | | | | |
| 205: | 1687 | 1632 | 4146 | 739 | 895 | 890 | 917 | 2761 | 898 | 2695 | 896 | 888 |
| | 902 | 883 | 3757 | 4317 | 4321 | 1638 | 4628 | 1238 | 1401 | 2002 | 1067 | 2838 |
| | 866 | 3141 | 4641 | 4161 | 4513 | 847 | 4640 | 3609 | 2489 | 3906 | 248 | 4638 |
| | 3684 | 1786 | 4651 | 1029 | 452 | 1801 | 2082 | 3579 | 2380 | 1665 | 2734 | 398 |
| | 3749 | 2915 | 3535 | 4469 | 1614 | 1654 | 4316 | 3990 | 1724 | 1472 | 4074 | 761 |
| | 2913 | 3368 | 1287 | 3554 | 3337 | 4650 | 2591 | 311 | 2182 | 3181 | 3285 | 1996 |
| | 2314 | 417 | 2881 | 507 | 3164 | 2291 | 1142 | 2577 | 374 | 1847 | 3038 | 1439 |
| | 851 | 3098 | 2464 | 4562 | 1529 | 3768 | 3921 | 3948 | 3110 | 4197 | 1135 | 3685 |
| | 3149 | 833 | 4230 | 3622 | 4659 | 1012 | 865 | 719 | 3469 | 2855 | 416 | 2841 |
| | 2782 | 1047 | 3735 | 1279 | 1782 | 2919 | 231 | 3349 | 1275 | 366 | 3000 | 4117 |
| | 2920 | 4220 | 3555 | 3812 | 2173 | 2170 | 2106 | 942 | 3691 | 4492 | 3638 | 1016 |
| | 1811 | 1474 | 2646 | 3674 | 3581 | 4182 | 1424 | 4388 | 692 | 4121 | 1134 | 1876 |
| | 4075 | 2767 | 970 | 652 | 4670 | 1621 | 3498 | 2906 | 1806 | 1199 | 2339 | 4642 |
| | 3843 | 3169 | 4495 | 4427 | 667 | 4092 | 2726 | 1823 | 2806 | 3536 | 2474 | 4024 |
| | 3924 | 4743 | 2147 | 2976 | 918 | 4587 | 1907 | 2958 | 3303 | 3693 | 3420 | 1283 |
| | 2502 | 258 | 4440 | 4123 | 1374 | 1560 | 770 | 422 | 4039 | 1908 | 469 | 1470 |
| | 1756 | 353 | 2199 | 1720 | 1729 | 1721 | 3723 | 4073 | 2514 | 2664 | 2512 | 1160 |
| | 2384 | 368 | 551 | 2675 | 2848 | 3153 | 3851 | 782 | 3182 | 483 | 1183 | 693 |
| | 2804 | 554 | 2081 | 730 | 1936 | 4720 | 3637 | 2618 | 3892 | 2595 | 3517 | 3616 |
| | 4444 | 3502 | 4062 | 1476 | 3426 | 4124 | 1105 | 1739 | 3095 | 2943 | 702 | 1414 |
| | 2068 | 979 | 683 | 246 | 2659 | 2334 | 810 | 1571 | 727 | 4458 | 558 | 3895 |
| | 726 | 4584 | 1733 | 4278 | 3742 | 1896 | 4537 | 2388 | 875 | 3093 | 925 | 941 |
| | 938 | 2554 | 940 | 1662 | 4467 | 4452 | 2868 | 4655 | 1223 | 3205 | 677 | 544 |
| | 2230 | 2227 | 569 | 548 | 564 | 566 | 3867 | 892 | 3869 | 2828 | 1639 | 2543 |
| | 2058 | 4071 | 626 | 2601 | 555 | 1269 | 2453 | 736 | 4480 | 3597 | 2409 | 2326 |
| | 3119 | 1150 | 521 | 3364 | 1140 | 658 | 4044 | 4399 | 4179 | 2932 | 2752 | 1685 |
| | 508 | 2172 | 3056 | 4740 | 4698 | 3857 | 3049 | 3134 | 774 | 3660 | 1232 | 2533 |
| | 1243 | 2946 | 486 | 1732 | 1244 | 3421 | 662 | 4459 | 1200 | 4684 | 3706 | 3344 |
| | 628 | 2698 | 575 | 3759 | 502 | 3552 | 3131 | 830 | 1063 | 3365 | 3897 | 724 |
| | 1400 | 4214 | 1620 | 1182 | 829 | 3894 | 1087 | 1218 | 2860 | 3681 | 371 | 2809 |
| | 4470 | 3214 | 2122 | 1290 | 519 | 3284 | 4348 | 3592 | 969 | 2207 | 4142 | 1902 |
| | 630 | 415 | 3248 | 4554 | 3519 | 771 | 3624 | 4302 | 4261 | 4239 | 4238 | 4260 |
| | 4254 | 4263 | 4242 | 4241 | 4240 | 4258 | 1526 | 4148 | 1543 | 4069 | 3840 | 3329 |
| | 2987 | 3938 | | | | | | | | | | |
| 206: | 685 | 1330 | 1241 | 1851 | 4788 | 2051 | 4617 | 2882 | 1755 | 4428 | 1108 | 3801 |
| | 2954 | 2565 | 1471 | 1485 | 4341 | 716 | 868 | 1879 | 1596 | 2921 | 4246 | 1494 |
| | 2850 | 1746 | 3672 | 3601 | | | | | | | | |
| 207: | 4106 | 2132 | 2509 | 3014 | 418 | 4609 | 2785 | 2904 | 633 | 779 | 2950 | 2338 |
| | 1532 | 3448 | 4629 | 4077 | 2259 | 2066 | 2041 | 1497 | 2302 | 2511 | 2682 | 1980 |
| | 787 | 2212 | 485 | 1138 | 4361 | 334 | 2936 | 1021 | 1003 | 1498 | 1099 | 1491 |
| | 2107 | 1852 | | | | | | | | | | |
| 208: | 4313 | 1862 | 1518 | 2097 | 2428 | 4144 | 347 | 4338 | 3604 | 2927 | 2117 | 4750 |
| | 4331 | 316 | 310 | 4534 | 229 | 4757 | 3140 | 2630 | 534 | 313 | 405 | 3986 |
| | 1710 | 1990 | 4314 | 1388 | 3881 | 2435 | 2972 | 4232 | 4176 | 639 | 923 | 2143 |
| | 2348 | 3810 | 2769 | 2389 | 2470 | | | | | | | |
| 209: | 4234 | 4337 | 329 | 4499 | 3666 | 533 | 3331 | 672 | 2200 | 4122 | 3710 | 3697 |
| | 3854 | 1499 | 3446 | 3413 | 1493 | 4653 | 257 | 3165 | 4712 | 2940 | | |
| 210: | 1147 | 265 | 476 | 1576 | 2033 | 594 | 3226 | 3198 | 1235 | 1340 | 4702 | 696 |
| | 1539 | 4585 | 4285 | 4364 | 4546 | 1590 | 2638 | 1208 | 4764 | 2713 | 1480 | 934 |
| | 2490 | 1477 | 2660 | 3262 | 2055 | 4370 | 407 | 2495 | 1749 | 3436 | 3476 | 2328 |
| | 3572 | 2282 | | | | | | | | | | |
| 211: | 1728 | 3229 | 3232 | 4292 | 3463 | 3479 | 2179 | 3412 | 4569 | 2981 | 2323 | 3221 |
| | 1699 | 1702 | 4200 | 2526 | 3247 | 3249 | 1829 | 4139 | 4141 | 3016 | 3017 | 1680 |
| | 4262 | 3227 | 3207 | 1969 | 4478 | 2416 | 4634 | 4293 | 1544 | 2478 | 1392 | 3083 |
| | 3377 | 867 | 3175 | 1697 | 1718 | 1677 | 1675 | 1503 | 2460 | 2392 | 2872 | 2874 |
| | 3047 | 4266 | 3771 | 1840 | 4733 | 1712 | 1690 | 1694 | 2871 | 3560 | 4201 | 3044 |
| | 1617 | 1619 | 2632 | 3832 | 1073 | 4333 | 794 | 791 | 784 | 2610 | 2604 | 2592 |
| | 2360 | 4149 | 2529 | 2550 | 2535 | 2531 | 3124 | 3491 | 462 | 4685 | 4221 | 1136 |
| | 1989 | 4610 | 1128 | 2463 | 2479 | 4304 | 4305 | 2978 | 3465 | 2979 | 3799 | 1329 |
| | 4550 | 1625 | 3975 | 2301 | 4746 | 4751 | 4748 | 4288 | 1643 | 1671 | 1646 | 1313 |
| | 4385 | 2244 | 2442 | 2239 | 1258 | 3932 | 4538 | 644 | 3453 | 3870 | 1901 | 4613 |
| | 2971 | 3325 | 2567 | 2575 | 431 | 4193 | 3206 | 3201 | 3203 | 2819 | 2590 | 4734 |
| | 2288 | 2537 | 2788 | 361 | 4790 | 3171 | 1966 | 2571 | 2568 | 4723 | 1316 | 2594 |
| | 3704 | 4150 | 3394 | 1783 | 1151 | 1155 | 1153 | 3252 | 3250 | 3472 | 1713 | 3283 |
| | 4524 | 3233 | 3018 | 3474 | 3107 | 839 | 840 | 842 | 2413 | 1350 | 3268 | 3287 |
| | 3680 | 1923 | 3423 | 1988 | 1606 | 1352 | 1711 | 1320 | 781 | 3492 | 4384 | 1751 |
| | 1993 | 4194 | 4776 | 4578 | 880 | 4391 | 4703 | 1115 | 3294 | 3435 | 4795 | 3841 |
| | 3914 | 2352 | 2481 | 3163 | 2959 | 2485 | 2484 | 1362 | 4127 | 3846 | 4747 | 3643 |
| | 4774 | 900 | 4752 | 3925 | 2219 | 242 | 1552 | 586 | 3080 | 3102 | 3082 | 3084 |
| | 3367 | 2043 | 2138 | 1593 | 2813 | 2342 | 1651 | 233 | 4815 | 2483 | 2482 | 2559 |
| | 2566 | 1121 | 2588 | 2589 | 2553 | 2551 | 3245 | 2614 | 2629 | 2607 | 2608 | 282 |
| | 302 | 2221 | 3847 | 3195 | 1831 | 2438 | 1788 | 1641 | 536 | 2304 | 538 | 3144 |
| | 1673 | 4264 | 1814 | 3886 | 3473 | 4639 | 4576 | 585 | | | | |
| 212: | 3386 | 1913 | 4531 | 3068 | 1473 | 1264 | 3279 | 4481 | 1796 | 1946 | 2791 | 3191 |
| | 1112 | | | | | | | | | | | |
| 213: | 4689 | 1448 | 151 | 3553 | 4631 | 4645 | 2609 | 4215 | 355 | 4484 | 587 | 429 |
| | 3114 | 1088 | 1076 | 3238 | 4724 | 2381 | 1304 | 2335 | 640 | | | |
| 214: | 3733 | 3732 | 3940 | 3074 | 2934 | 3999 | 3409 | 4623 | 2136 | 3868 | 4686 | 2133 |
| | 4563 | 4583 | 4557 | 2859 | 4561 | 2861 | 4586 | 4621 | 3063 | 475 | 1858 | 2040 |
| | 237 | 2283 | 3199 | 3998 | 3569 | 4597 | 3026 | 3567 | 4600 | 4543 | 2258 | 3884 |
| | 3911 | 3888 | 4630 | 2181 | 308 | 1968 | 4672 | 2086 | 2189 | 814 | 424 | 3626 |
| | 4111 | 1761 | 3258 | 780 | 2359 | 611 | 2935 | 2975 | 3457 | 4023 | 2131 | 4620 |
| | 3563 | 807 | 4101 | 788 | 2044 | 627 | 765 | 763 | 2075 | 4581 | 2069 | 2047 |
| | 4565 | 2096 | 4120 | 4656 | 828 | 805 | 804 | 4248 | 2938 | 3059 | 2974 | 4003 |
| | 3104 | 2977 | 3829 | 1165 | | | | | | | | |
| 215: | 4048 | 678 | 4464 | 802 | 645 | 4181 | 4652 | 2358 | 325 | 3204 | 670 | 3125 |
| | 4644 | 2157 | 3405 | 3266 | 4536 | 4058 | 510 | 1181 | 4692 | | | |
| 216: | 681 | 2506 | 819 | 2680 | 1430 | 1209 | 4217 | 3373 | 1541 | 2715 | 596 | 2317 |
| | 2459 | 2355 | 2611 | 3570 | 1384 | 1931 | 983 | 3741 | 1423 | 1958 | 4297 | 4512 |
| | 2439 | 4498 | 2393 | 2363 | | | | | | | | |
| 217: | 3320 | 3385 | 4000 | 2693 | 2687 | 2690 | 4718 | 378 | 412 | 2684 | 3814 | 4555 |
| | 352 | 1337 | 2562 | 1311 | 610 | 1098 | 3179 | 3178 | 4592 | 4591 | 2319 | 1089 |
| | 3489 | 2454 | 1611 | 4276 | 4034 | 1033 | 601 | 4377 | 4375 | 4663 | 1853 | 1856 |
| | 3388 | 2466 | 2993 | 514 | 3910 | 1169 | 3395 | 4154 | 1133 | 4446 | 4420 | 3753 |
| | 3797 | 1895 | 389 | 2145 | 4410 | 4407 | 4443 | 4425 | 3157 | 286 | 735 | 2144 |
| | 3340 | 754 | 4445 | 3774 | 342 | 337 | 340 | 3417 | 3012 | 1300 | 4099 | 4549 |
| | 4309 | 549 | 4195 | 3351 | 4624 | 3151 | 2256 | 4730 | 1130 | 430 | 4674 | 612 |
| | 1101 | 4599 | 2837 | 2783 | 721 | 846 | 4372 | 4360 | 511 | 4340 | 3796 | |
| 218: | 4691 | 4466 | 3495 | 4116 | 4727 | 740 | 4539 | 3025 | 1282 | 3945 | 4472 | 3942 |
| | 570 | 2790 | 1963 | 576 | 2816 | 2705 | 1657 | | | | | |
| 219: | 1973 | 3315 | 3347 | 245 | 1178 | 1518 | 3330 | 3760 | 4349 | 1156 | 1707 | 1251 |
| | 751 | 600 | 2331 | 955 | 1930 | 1915 | 2480 | 2374 | 4338 | 497 | 1917 | 1918 |
| | 1916 | 270 | 1189 | 281 | 3372 | 2574 | 316 | 310 | 232 | 4755 | 3140 | 313 |
| | 405 | 2944 | 4580 | 4255 | 3286 | 3654 | 2603 | 552 | 599 | 923 | 4520 | 2444 |
| | 2396 | 759 | 1727 | 3964 | | | | | | | | |
| 220: | 4327 | 1635 | 1850 | 4282 | 4433 | 3860 | 1698 | 2717 | 2918 | 3650 | 1305 | 2724 |
| | 706 | 4209 | 4021 | | | | | | | | | |
| 221: | 1001 | 3296 | 1845 | 3522 | 579 | 4353 | 2956 | 3740 | 4589 | 1132 | 1566 | 2557 |
| | 935 | 1137 | 669 | 2126 | 3726 | 2685 | 3707 | 919 | 4189 | 2092 | 4093 | 2391 |
| | 269 | | | | | | | | | | | |
| 222: | 1973 | 4315 | 4313 | 4627 | 3350 | 1802 | 531 | 1709 | 2123 | 3617 | 647 | 2480 |
| | 2374 | 347 | 4338 | 3604 | 3993 | 3734 | 4067 | 2174 | 1215 | 1495 | 1776 | 229 |
| | 4534 | 4757 | 4781 | 1672 | 3140 | 2630 | 2662 | 313 | 2290 | 1995 | 4303 | 2617 |
| | 3679 | 3480 | 3470 | 2364 | 2666 | 2455 | 3060 | 4700 | 2735 | 2348 | 2444 | 2389 |
| | 759 | 2740 | 2835 | 2729 | 3821 | | | | | | | |
| 223: | 2494 | 4569 | 4051 | 1699 | 1702 | 3241 | 2525 | 4430 | 1680 | 251 | 2994 | 1482 |
| | 1479 | 2416 | 2415 | 4634 | 1544 | 2478 | 1392 | 2419 | 2424 | 3376 | 3175 | 1718 |
| | 1697 | 1715 | 1677 | 1675 | 4643 | 4570 | 1503 | 2460 | 4733 | 1690 | 1712 | 1694 |
| | 2871 | 3560 | 4280 | 4201 | 3044 | 3076 | 2634 | 2637 | 791 | 784 | 813 | 767 |
| | 2604 | 2360 | 2529 | 3124 | 1113 | 3491 | 250 | 252 | 462 | 4685 | 1989 | 855 |
| | 1486 | 3799 | 1426 | 4156 | 4550 | 3975 | 2301 | 2814 | 1671 | 1643 | 1646 | 1844 |
| | 3669 | 644 | 3490 | 3453 | 4208 | 3471 | 1618 | 1901 | 758 | 1356 | 3053 | 756 |
| | 1281 | 3823 | 3826 | 2177 | 1783 | 3472 | 1713 | 3264 | 3283 | 3263 | 1507 | 3348 |
| | 3467 | 2401 | 1922 | 3029 | 646 | 4802 | 4211 | 3496 | 3497 | 4798 | 515 | 1177 |
| | 4366 | 3803 | 1457 | 394 | 1743 | 4711 | 3914 | 2352 | 2957 | 3163 | 2485 | 2219 |
| | 2810 | 2813 | 2812 | 1651 | 4815 | 1948 | 3437 | 2559 | 1121 | 2438 | 2440 | 1741 |
| | 1641 | 1673 | 3473 | 2556 | | | | | | | | |
| 224: | 2295 | 4094 | 4525 | 1885 | 1558 | 396 | 708 | 333 | 331 | 2203 | | |
| 225: | 4787 | 1413 | 1023 | 358 | 1521 | 1061 | 1312 | 4449 | 4130 | | | |
| 226: | 4709 | 3375 | 922 | 926 | 2077 | 3218 | | | | | | |
| 227: | 369 | 4098 | 4367 | 1649 | 3133 | 3120 | 547 | 1489 | 2229 | 2027 | 591 | 4604 |
| | 757 | 238 | | | | | | | | | | |
| 228: | 3434 | 1991 | 2185 | 3187 | 4155 | 4775 | 2294 | 3032 | 2911 | 1469 | 4105 | 4110 |
| | 4125 | 4107 | 4109 | 3652 | 4126 | 2692 | 4771 | 2487 | 688 | 2877 | 4770 | |

### Example 3

### Consensus Sequence Build

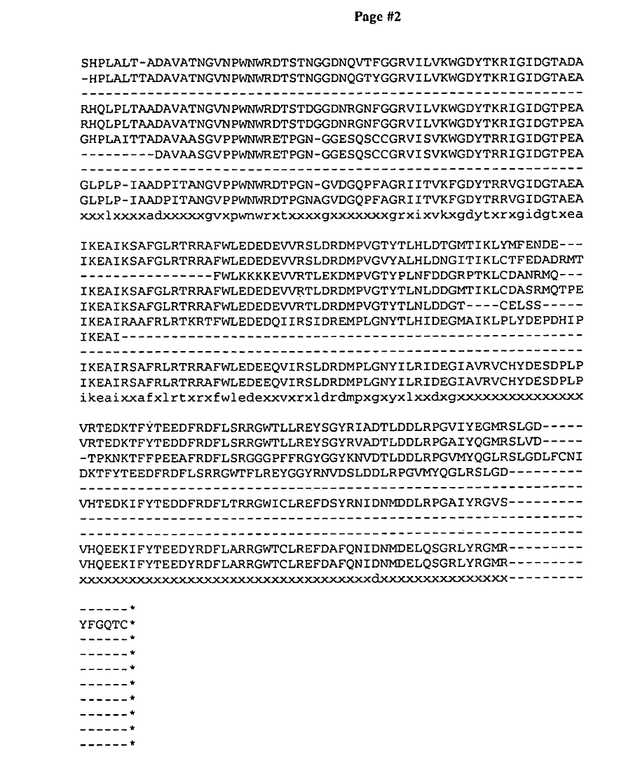

ClustalW program is selected for multiple sequence alignments of an amino acid sequence of SEQ ID NO: 115 and its homologs, through SEQ ID NO: 228 and its homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment. The consensus sequence of SEQ ID NO: 181 and its 9 homologs were derived according to the procedure described above and is displayed in FIG. 4.

### Example 4

### Pfam Module Annotation

This example illustrates the identification of domain and domain module by Pfam analysis. The amino acid sequence of the expressed proteins that were shown to be associated with an enhanced trait were analyzed for Pfam protein family against the current Pfam collection of multiple sequence alignments and hidden Markov models using the HMMER software in the appended computer listing. The Pfam domain modules and individual protein domain for the proteins of SEQ ID NO: 115 through 228 are shown in Table 17 and Table 18 respectively. The Hidden Markov model databases for the identified patent families are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains. For instance, the protein with amino acids of SEQ ID NO: 118 is characterized by two Pfam domains, i.e. “F-box” and “Tub”. See also the protein with amino acids of SEQ ID NO: 166 which is characterized by two copies of the Pfam domain “Myb_DNA-binding”. In Table 18 “score” is the gathering score for the Hidden Markov Model of the domain which exceeds the gathering cutoff reported in Table 19.

TABLE 17

| PEP | | | |
|---|---|---|---|
| Seq | Construct | | |
| ID | ID | Pfam Module | Position |
| 115 | CGPG110 | AP2 | 79-144 |
| 116 | CGPG1135 | zf-C2H2 | 126-149 |
| 117 | CGPG1180 | PLATZ | 30-141 |
| 118 | CGPG1197 | F-box::Tub | 31-87::98-380 |
| 119 | CGPG1802 | zf-C3HC4::YDG_SRA::zf-C3HC4 | 129-168::253-411::495-551 |
| 120 | CGPG2561 | SBP | 62-140 |
| 121 | CGPG2580 | HLH | 62-114 |
| 122 | CGPG2582 | AP2 | 42-106 |
| 123 | CGPG2591 | zf-B_box::zf-B_box | 1-47::58-101 |
| 124 | CGPG2597 | zf-C3HC4 | 111-152 |
| 125 | CGPG2602 | AP2 | 133-197 |
| 126 | CGPG2642 | HMG_box::HMG_box::HMG_box | 138-206::255-321::379-447 |
| 127 | CGPG2651 | zf-C3HC4 | 102-143 |
| 128 | CGPG2662 | zf-C2H2 | 152-175 |
| 129 | CGPG2708 | GATA | 223-258 |
| 130 | CGPG2710 | HLH | 211-261 |
| 131 | CGPG2722 | AP2 | 68-132 |
| 132 | CGPG2736 | NAM | 52-181 |
| 133 | CGPG2759 | zf-Dof | 143-205 |
| 134 | CGPG2765 | WRKY | 66-125 |
| 135 | CGPG2802 | AP2 | 42-106 |
| 136 | CGPG2823 | HMG_box | 193-260 |
| 137 | CGPG2889 | zf-CCCH::KH_1::zf-CCCH | 21-43::95-157::186-211 |
| 138 | CGPG2902 | SRF-TF | 14-64 |
| 139 | CGPG2913 | WRKY | 222-281 |
| 140 | CGPG2945 | zf-C3HC4 | 117-158 |
| 141 | CGPG2962 | zf-Dof | 35-97 |
| 142 | CGPG2967 | zf-Dof | 20-82 |
| 143 | CGPG2983 | AP2 | 94-158 |
| 144 | CGPG2996 | AP2 | 83-148 |
| 145 | CGPG3307 | DUF248 | 95-605 |
| 146 | CGPG3311 | zf-C2H2 | 89-111 |
| 147 | CGPG3350 | SRF-TF::K-box | 9-59::75-173 |
| 148 | CGPG3355 | zf-Dof | 24-86 |
| 149 | CGPG3447 | zf-C2H2 | 117-140 |
| 150 | CGPG3453 | Myb_DNA-binding::Myb_DNA-binding | 13-59::65-110 |
| 152 | CGPG3455 | Myb_DNA-binding::Myb_DNA- | 75-121::127-173::179-224 |
| | | binding::Myb_DNA-binding | |
| 153 | CGPG3488 | SRF-TF | 9-59 |
| 154 | CGPG353 | Pex2_Pex12::zf-C3HC4 | 41-270::327-364 |
| 155 | CGPG3537 | bZIP_2 | 68-122 |
| 156 | CGPG355 | HLH | 309-359 |
| 157 | CGPG3751 | GRAS | 154-454 |
| 158 | CGPG3755 | Myb_DNA-binding | 107-157 |
| 159 | CGPG3756 | GRAS | 119-426 |
| 160 | CGPG3757 | F-box | 38-85 |
| 161 | CGPG3776 | GRAS | 118-437 |
| 162 | CGPG3778 | WRKY | 109-171 |
| 163 | CGPG3799 | AT_hook::DUF296 | 76-88::160-280 |
| 164 | CGPG3822 | GRAS | 248-550 |
| 165 | CGPG3890 | Myb_DNA-binding::Myb_DNA-binding | 105-151::157-202 |
| 166 | CGPG3965 | Myb_DNA-binding::Myb_DNA-binding | 17-66::72-117 |
| 167 | CGPG3966 | HLH | 74-125 |
| 168 | CGPG3974 | zf-C2H2 | 94-116 |
| 169 | CGPG3977 | NAM | 11-135 |
| 170 | CGPG3984 | zf-B_box::zf-B_box | 1-47::52-99 |
| 171 | CGPG3988 | Myb_DNA-binding | 98-145 |
| 172 | CGPG4068 | NAM | 7-132 |
| 173 | CGPG4105 | PHD | 198-248 |
| 174 | CGPG4109 | SRF-TF::K-box | 9-59::75-175 |
| 175 | CGPG4125 | zf-C3HC4 | 156-196 |
| 176 | CGPG4126 | HLH | 129-178 |
| 177 | CGPG4167 | SRF-TF::K-box | 9-59::71-173 |
| 178 | CGPG4176 | Myb_DNA-binding | 9-58 |
| 179 | CGPG4193 | WRKY | 243-303 |
| 180 | CGPG4210 | SRF-TF::K-box | 9-59::78-174 |
| 182 | CGPG4540 | Myb_DNA-binding | 94-141 |
| 184 | CGPG4571 | Myb_DNA-binding::Linker_histone | 5-57::126-201 |
| 185 | CGPG4622 | Myb_DNA-binding::Myb_DNA-binding | 12-59::65-110 |
| 186 | CGPG4700 | Myb_DNA-binding::Myb_DNA-binding | 5-56::134-181 |
| 187 | CGPG475 | bZIP_1 | 89-142 |
| 188 | CGPG478 | Myb_DNA-binding::Myb_DNA-binding | 14-61::67-112 |
| 189 | CGPG497 | SRF-TF::K-box | 9-59::74-173 |
| 190 | CGPG5283 | SRF-TF::K-box | 25-75::88-189 |
| 191 | CGPG5284 | CBFD_NEYB_HMF | 102-166 |
| 192 | CGPG5294 | AUX_IAA | 11-192 |
| 193 | CGPG5308 | Myb_DNA-binding::Myb_DNA-binding | 11-60::117-164 |
| 194 | CGPG5322 | LIM::LIM | 11-68::110-167 |
| 195 | CGPG5427 | IBR | 208-271 |
| 196 | CGPG5595 | SET | 746-874 |
| 197 | CGPG5806 | bZIP_1 | 169-233 |
| 198 | CGPG626 | Mov34 | 54-168 |
| 199 | CGPG6434 | ZZ::Myb_DNA-binding::SWIRM | 1-46::62-108::349-434 |
| 200 | CGPG688 | bZIP_1 | 187-245 |
| 201 | CGPG7334 | E2F_TDP::E2F_TDP | 12-77::148-224 |
| 202 | CGPG7339 | Myb_DNA-binding | 5-57 |
| 203 | CGPG7350 | Prefoldin | 24-143 |
| 204 | CGPG7475 | NAM | 18-147 |
| 205 | CGPG7493 | HD-ZIP_N::Homeobox::HALZ | 1-115::141-195::196-240 |
| 207 | CGPG7598 | Myb_DNA-binding::Myb_DNA-binding | 30-79::136-183 |
| 208 | CGPG7600 | AP2 | 20-84 |
| 209 | CGPG7604 | GATA | 212-247 |
| 210 | CGPG7615 | zf-C3HC4 | 150-187 |
| 211 | CGPG7630 | SRF-TF::K-box | 9-59::75-174 |
| 212 | CGPG7631 | bZIP_1 | 121-178 |
| 214 | CGPG7644 | TCP | 38-253 |
| 215 | CGPG7691 | zf-C3HC4 | 30-76 |
| | | | 77-112::114-149:: |
| 216 | CGPG7696 | Ank::Ank::zf-CCCH::zf-CCCH | 270-295::305-327 |
| 217 | CGPG7703 | Myb_DNA-binding::Myb_DNA-binding | 14-61::67-112 |
| 218 | CGPG7707 | TCP | 16-209 |
| 219 | CGPG7731 | AP2 | 111-176 |
| 220 | CGPG7734 | ZZ | 307-360 |
| 221 | CGPG7753 | zf-C2H2::zf-C2H2 | 62-84::142-164 |
| 222 | CGPG7836 | AP2 | 23-87 |
| 223 | CGPG7867 | SRF-TF::K-box | 9-59::85-175 |
| 224 | CGPG8105 | B3 | 128-225 |
| 225 | CGPG8132 | zf-LSD1::zf-LSD1 | 63-87::101-125 |
| 227 | CGPG8175 | HLH | 86-135 |

TABLE 18

| PEP | Con- | Pfam | | | | |
|---|---|---|---|---|---|---|
| Seq | struct | domain | | | | |
| ID No. | ID | name | begin | start | score | E-value |
| 115 | CGPG110 | AP2 | 79 | 144 | 152.7 | 9.50E−43 |
| 116 | CGPG1135 | zf-C2H2 | 126 | 149 | 19.8 | 0.0097 |
| 117 | CGPG1180 | PLATZ | 30 | 141 | 288.2 | 1.60E−83 |
| 118 | CGPG1197 | F-box | 31 | 87 | 36.1 | 1.20E−07 |
| 118 | CGPG1197 | Tub | 98 | 380 | 627.7 | 1.00E−185 |
| 119 | CGPG1802 | zf-C3HC4 | 129 | 168 | 36.9 | 6.90E−08 |
| 119 | CGPG1802 | YDG_SRA | 253 | 411 | 259.6 | 6.20E−75 |
| 119 | CGPG1802 | zf-C3HC4 | 495 | 551 | 28.9 | 1.70E−05 |
| 120 | CGPG2561 | SBP | 62 | 140 | 187.5 | 3.10E−53 |
| 121 | CGPG2580 | HLH | 62 | 114 | 67 | 6.30E−17 |
| 122 | CGPG2582 | AP2 | 42 | 106 | 148.8 | 1.50E−41 |
| 123 | CGPG2591 | zf-B_box | 1 | 47 | 39.8 | 9.50E−09 |
| 123 | CGPG2591 | zf-B_box | 58 | 101 | 44.3 | 4.20E−10 |
| 124 | CGPG2597 | zf-C3HC4 | 111 | 152 | 36.5 | 9.20E−08 |
| 125 | CGPG2602 | AP2 | 133 | 197 | 138.5 | 1.80E−38 |
| 126 | CGPG2642 | HMG_box | 138 | 206 | 71.4 | 2.90E−18 |
| 126 | CGPG2642 | HMG_box | 255 | 321 | 84.2 | 3.90E−22 |
| 126 | CGPG2642 | HMG_box | 379 | 447 | 77.6 | 4.00E−20 |
| 127 | CGPG2651 | zf-C3HC4 | 102 | 143 | 39.9 | 9.00E−09 |
| 128 | CGPG2662 | zf-C2H2 | 152 | 175 | 20.1 | 0.0078 |
| 129 | CGPG2708 | GATA | 223 | 258 | 67.8 | 3.50E−17 |
| 130 | CGPG2710 | HLH | 211 | 261 | 32.6 | 1.40E−06 |
| 131 | CGPG2722 | AP2 | 68 | 132 | 138.3 | 2.10E−38 |
| 132 | CGPG2736 | NAM | 52 | 181 | 56.9 | 6.80E−14 |
| 133 | CGPG2759 | zf-Dof | 143 | 205 | 132.1 | 1.60E−36 |
| 134 | CGPG2765 | WRKY | 66 | 125 | 141.6 | 2.10E−39 |
| 135 | CGPG2802 | AP2 | 42 | 106 | 135.2 | 1.80E−37 |
| 136 | CGPG2823 | HMG_box | 193 | 260 | 32.9 | 1.20E−06 |
| 137 | CGPG2889 | zf-CCCH | 21 | 43 | 4.4 | 0.35 |
| 137 | CGPG2889 | KH_1 | 95 | 157 | 58.8 | 1.70E−14 |
| 137 | CGPG2889 | zf-CCCH | 186 | 211 | 46 | 1.30E−10 |
| 138 | CGPG2902 | SRF-TF | 14 | 64 | 29.5 | 1.20E−05 |
| 139 | CGPG2913 | WRKY | 222 | 281 | 144.4 | 3.00E−40 |
| 140 | CGPG2945 | zf-C3HC4 | 117 | 158 | 21.3 | 0.00098 |
| 141 | CGPG2962 | zf-Dof | 35 | 97 | 134.3 | 3.40E−37 |
| 142 | CGPG2967 | zf-Dof | 20 | 82 | 126.1 | 9.60E−35 |
| 143 | CGPG2983 | AP2 | 94 | 158 | 75.7 | 1.50E−19 |
| 144 | CGPG2996 | AP2 | 83 | 148 | 137.1 | 4.90E−38 |
| 145 | CGPG3307 | DUF248 | 95 | 605 | 1274.5 | 0 |
| 146 | CGPG3311 | zf-C2H2 | 89 | 111 | 23 | 0.001 |
| 147 | CGPG3350 | SRF-TF | 9 | 59 | 106.1 | 1.00E−28 |
| 147 | CGPG3350 | K-box | 75 | 173 | 108.1 | 2.60E−29 |
| 148 | CGPG3355 | zf-Dof | 24 | 86 | 139.1 | 1.20E−38 |
| 149 | CGPG3447 | zf-C2H2 | 117 | 140 | 20.2 | 0.0074 |
| 150 | CGPG3453 | Myb_DNA-binding | 13 | 59 | 60 | 7.50E−15 |
| 150 | CGPG3453 | Myb_DNA-binding | 65 | 110 | 48.9 | 1.70E−11 |
| 152 | CGPG3455 | Myb_DNA-binding | 75 | 121 | 54.7 | 3.00E−13 |
| 152 | CGPG3455 | Myb_DNA-binding | 127 | 173 | 59.3 | 1.30E−14 |
| 152 | CGPG3455 | Myb_DNA-binding | 179 | 224 | 44.6 | 3.40E−10 |
| 153 | CGPG3488 | SRF-TF | 9 | 59 | 66.2 | 1.00E−16 |
| 154 | CGPG353 | Pex2_Pex12 | 41 | 270 | 288.3 | 1.40E−83 |
| 154 | CGPG353 | zf-C3HC4 | 327 | 364 | 29.3 | 1.30E−05 |
| 155 | CGPG3537 | bZIP_1 | 68 | 132 | 28.3 | 2.80E−05 |
| 155 | CGPG3537 | bZIP_2 | 68 | 122 | 47.8 | 3.70E−11 |
| 156 | CGPG355 | HLH | 309 | 359 | 32.8 | 1.20E−06 |
| 157 | CGPG3751 | GRAS | 154 | 454 | 524.7 | 1.00E−154 |
| 158 | CGPG3755 | Myb_DNA-binding | 107 | 157 | 43.6 | 6.50E−10 |
| 159 | CGPG3756 | GRAS | 119 | 426 | 385.6 | 7.60E−113 |
| 160 | CGPG3757 | F-box | 38 | 85 | 43.3 | 8.20E−10 |
| 161 | CGPG3776 | GRAS | 118 | 437 | 450.8 | 1.80E−132 |
| 162 | CGPG3778 | WRKY | 109 | 171 | 93.5 | 6.60E−25 |
| 163 | CGPG3799 | AT_hook | 76 | 88 | 19.1 | 0.012 |
| 163 | CGPG3799 | DUF296 | 160 | 280 | 218.5 | 1.50E−62 |
| 164 | CGPG3822 | GRAS | 248 | 550 | 469.7 | 3.70E−138 |
| 165 | CGPG3890 | Myb_DNA-binding | 105 | 151 | 54.9 | 2.70E−13 |
| 165 | CGPG3890 | Myb_DNA-binding | 157 | 202 | 46.9 | 6.80E−11 |
| 166 | CGPG3965 | Myb_DNA-binding | 17 | 66 | 44.9 | 2.80E−10 |
| 166 | CGPG3965 | Myb_DNA-binding | 72 | 117 | 39.8 | 9.30E−09 |
| 167 | CGPG3966 | HLH | 74 | 125 | 39.7 | 9.70E−09 |
| 168 | CGPG3974 | zf-C2H2 | 94 | 116 | 22.4 | 0.0016 |
| 169 | CGPG3977 | NAM | 11 | 135 | 260.6 | 3.10E−75 |
| 170 | CGPG3984 | zf-B_box | 1 | 47 | 51 | 4.00E−12 |
| 170 | CGPG3984 | zf-B_box | 52 | 99 | 58.5 | 2.20E−14 |
| 171 | CGPG3988 | Myb_DNA-binding | 98 | 145 | 40.9 | 4.30E−09 |
| 172 | CGPG4068 | NAM | 7 | 132 | 301.1 | 2.10E−87 |
| 173 | CGPG4105 | PHD | 198 | 248 | 56.1 | 1.10E−13 |
| 174 | CGPG4109 | SRF-TF | 9 | 59 | 116.6 | 7.00E−32 |
| 174 | CGPG4109 | K-box | 75 | 175 | 147.1 | 4.50E−41 |
| 175 | CGPG4125 | zf-C3HC4 | 156 | 196 | 27.9 | 3.50E−05 |
| 176 | CGPG4126 | HLH | 129 | 178 | 35.9 | 1.40E−07 |
| 177 | CGPG4167 | SRF-TF | 9 | 59 | 113.7 | 5.40E−31 |
| 177 | CGPG4167 | K-box | 71 | 173 | 74.5 | 3.40E−19 |
| 178 | CGPG4176 | Myb_DNA-binding | 9 | 58 | 31.5 | 2.90E−06 |
| 179 | CGPG4193 | WRKY | 243 | 303 | 143.4 | 6.20E−40 |
| 180 | CGPG4210 | SRF-TF | 9 | 59 | 104.2 | 3.70E−28 |
| 180 | CGPG4210 | K-box | 78 | 174 | 44.1 | 4.60E−10 |
| 182 | CGPG4540 | Myb_DNA-binding | 94 | 141 | 47.2 | 5.70E−11 |
| 184 | CGPG4571 | Myb_DNA-binding | 5 | 57 | 33.4 | 8.20E−07 |
| 184 | CGPG4571 | Linker_histone | 126 | 201 | 25.8 | 2.60E−05 |
| 185 | CGPG4622 | Myb_DNA-binding | 12 | 59 | 40.5 | 5.60E−09 |
| 185 | CGPG4622 | Myb_DNA-binding | 65 | 110 | 41.6 | 2.70E−09 |
| 186 | CGPG4700 | Myb_DNA-binding | 5 | 56 | 39.7 | 1.00E−08 |
| 186 | CGPG4700 | Myb_DNA-binding | 134 | 181 | 43.9 | 5.50E−10 |
| 187 | CGPG475 | bZIP_1 | 89 | 142 | 28.7 | 2.00E−05 |
| 187 | CGPG475 | bZIP_2 | 89 | 139 | 17.9 | 0.011 |
| 188 | CGPG478 | Myb_DNA-binding | 14 | 61 | 48.8 | 1.90E−11 |
| 188 | CGPG478 | Myb_DNA-binding | 67 | 112 | 48.5 | 2.30E−11 |
| 189 | CGPG497 | SRF-TF | 9 | 59 | 121 | 3.30E−33 |
| 189 | CGPG497 | K-box | 74 | 173 | 150.5 | 4.60E−42 |
| 190 | CGPG5283 | SRF-TF | 25 | 75 | 118.8 | 1.60E−32 |
| 190 | CGPG5283 | K-box | 88 | 189 | 160 | 6.00E−45 |
| 191 | CGPG5284 | Histone | 96 | 169 | 53.2 | 8.70E−13 |
| 191 | CGPG5284 | CBFD_NFYB_HMF | 102 | 166 | 93.4 | 6.90E−25 |
| 192 | CGPG5294 | AUX_IAA | 11 | 192 | 391.1 | 1.60E−114 |
| 193 | CGPG5308 | Myb_DNA-binding | 11 | 60 | 22.5 | 0.0016 |
| 193 | CGPG5308 | Myb_DNA-binding | 117 | 164 | 48.6 | 2.10E−11 |
| 194 | CGPG5322 | LIM | 11 | 68 | 53.4 | 7.70E−13 |
| 194 | CGPG5322 | LIM | 110 | 167 | 63.9 | 5.20E−16 |
| 195 | CGPG5427 | IBR | 208 | 271 | 85 | 2.30E−22 |
| 196 | CGPG5595 | SET | 746 | 874 | 159.9 | 6.70E−45 |
| 197 | CGPG5806 | bZIP_1 | 169 | 233 | 28.2 | 2.80E−05 |
| 197 | CGPG5806 | bZIP_2 | 169 | 223 | 24.7 | 0.00033 |
| 198 | CGPG626 | Mov34 | 54 | 168 | 178.6 | 1.50E−50 |
| 199 | CGPG6434 | ZZ | 1 | 46 | 85.2 | 2.00E−22 |
| 199 | CGPG6434 | Myb_DNA-binding | 62 | 108 | 48.1 | 3.10E−11 |
| 199 | CGPG6434 | SWIRM | 349 | 434 | 83.2 | 7.90E−22 |
| 200 | CGPG688 | bZIP_1 | 187 | 245 | 46 | 1.30E−10 |
| 200 | CGPG688 | bZIP_2 | 188 | 241 | 44.6 | 3.30E−10 |
| 201 | CGPG7334 | E2F_TDP | 12 | 77 | 115.1 | 2.00E−31 |
| 201 | CGPG7334 | E2F_TDP | 148 | 224 | 119 | 1.30E−32 |
| 202 | CGPG7339 | Myb_DNA-binding | 5 | 57 | 35 | 2.60E−07 |
| 203 | CGPG7350 | Prefoldin | 24 | 143 | 126.1 | 9.60E−35 |
| 204 | CGPG7475 | NAM | 18 | 147 | 257.9 | 2.00E−74 |
| 205 | CGPG7493 | HD-ZIP_N | 1 | 115 | 139.2 | 1.10E−38 |
| 205 | CGPG7493 | Homeobox | 141 | 195 | 72 | 1.90E−18 |
| 205 | CGPG7493 | HALZ | 196 | 240 | 89.5 | 1.00E−23 |
| 207 | CGPG7598 | Myb_DNA-binding | 30 | 79 | 48.6 | 2.10E−11 |
| 207 | CGPG7598 | Myb_DNA-binding | 136 | 183 | 49.6 | 1.10E−11 |
| 208 | CGPG7600 | AP2 | 20 | 84 | 143.2 | 6.90E−40 |
| 209 | CGPG7604 | GATA | 212 | 247 | 67.2 | 5.30E−17 |
| 210 | CGPG7615 | zf-C3HC4 | 150 | 187 | 33.4 | 7.70E−07 |
| 211 | CGPG7630 | SRF-TF | 9 | 59 | 111.6 | 2.20E−30 |
| 211 | CGPG7630 | K-box | 75 | 174 | 157.8 | 2.80E−44 |
| 212 | CGPG7631 | bZIP_1 | 121 | 178 | 30.1 | 7.80E−06 |
| 212 | CGPG7631 | bZIP_2 | 121 | 175 | 26.1 | 0.00013 |
| 214 | CGPG7644 | TCP | 38 | 253 | 139.2 | 1.10E−38 |
| 215 | CGPG7691 | zf-C3HC4 | 30 | 76 | 28.2 | 2.90E−05 |
| 216 | CGPG7696 | Ank | 77 | 112 | 13.1 | 0.4 |
| 216 | CGPG7696 | Ank | 114 | 149 | 31.7 | 2.60E−06 |
| 216 | CGPG7696 | zf-CCCH | 270 | 295 | 27.2 | 6.00E−05 |
| 216 | CGPG7696 | zf-CCCH | 305 | 327 | 0.4 | 1.2 |
| 217 | CGPG7703 | Myb_DNA-binding | 14 | 61 | 51.5 | 2.80E−12 |
| 217 | CGPG7703 | Myb_DNA-binding | 67 | 112 | 34.7 | 3.10E−07 |
| 218 | CGPG7707 | TCP | 16 | 209 | 149.1 | 1.20E−41 |
| 219 | CGPG7731 | AP2 | 111 | 176 | 146.8 | 5.70E−41 |
| 220 | CGPG7734 | ZZ | 307 | 360 | 27 | 6.50E−05 |
| 221 | CGPG7753 | zf-C2H2 | 62 | 84 | 27.3 | 5.30E−05 |
| 221 | CGPG7753 | zf-C2H2 | 142 | 164 | 23.6 | 0.00071 |
| 222 | CGPG7836 | AP2 | 23 | 87 | 134.4 | 3.20E−37 |
| 223 | CGPG7867 | SRF-TF | 9 | 59 | 95.2 | 1.90E−25 |
| 223 | CGPG7867 | K-box | 85 | 175 | 22.5 | 5.60E−06 |
| 224 | CGPG8105 | B3 | 128 | 225 | 82.2 | 1.60E−21 |
| 225 | CGPG8132 | zf-LSD1 | 63 | 87 | 50.1 | 7.30E−12 |
| 225 | CGPG8132 | zf-LSD1 | 101 | 125 | 54.5 | 3.50E−13 |
| 227 | CGPG8175 | HLH | 86 | 135 | 32.2 | 1.80E−06 |

TABLE 19

| | Accession | gathering | |
|---|---|---|---|
| Pfam domain name | # | cutoff | domain description |
| AT_hook | PF02178.9 | 3.6 | AT hook motif |
| AUX_IAA | PF02309.7 | −83 | AUX/IAA family |
| Ank | PF00023.20 | 0 | Ankyrin repeat |
| B3 | PF02362.12 | 26.5 | B3 DNA binding domain |
| CBFD_NFYB_HMF | PF00808.13 | 18.4 | Histone-like transcription factor (CBF/NF-Y) and |
| | | | archaeal histone |
| DUF248 | PF03141.7 | −363.8 | Putative methyltransferase |
| DUF296 | PF03479.5 | −11 | Domain of unknown function (DUF296) |
| E2F_TDP | PF02319.11 | 17 | E2F/DP family winged-helix DNA-binding domain |
| F-box | PF00646.23 | 13.9 | F-box domain |
| GATA | PF00320.17 | 28.5 | GATA zinc finger |
| GRAS | PF03514.5 | −78 | GRAS family transcription factor |
| HALZ | PF02183.8 | 17 | Homeobox associated leucine zipper |
| HD-ZIP_N | PF04618.3 | 25 | HD-ZIP protein N terminus |
| HLH | PF00010.16 | 8.3 | Helix-loop-helix DNA-binding domain |
| HMG_box | PF00505.9 | 4.1 | HMG (high mobility group) box |
| Histone | PF00125.14 | 17.4 | Core histone H2A/H2B/H3/H4 |
| Homeobox | PF00046.19 | −4.1 | Homeobox domain |
| IBR | PF01485.11 | 0 | IBR domain |
| K-box | PF01486.8 | 0 | K-box region |
| KH_1 | PF00013.19 | 10.5 | KH domain |
| LIM | PF00412.12 | 0 | LIM domain |
| Linker_histone | PF00538.9 | −8 | linker histone H1 and H5 family |
| Mov34 | PF01398.11 | −4 | Mov34/MPN/PAD-1 family |
| Myb_DNA-binding | PF00249.21 | 14 | Myb-like DNA-binding domain |
| NAM | PF02365.6 | −19 | No apical meristem (NAM) protein |
| PHD | PF00628.19 | 25.9 | PHD-finger |
| PLATZ | PF04640.4 | 20 | PLATZ transcription factor |
| Pex2_Pex12 | PF04757.5 | −50 | Pex2/Pex12 amino terminal region |
| Prefold in | PF02996.8 | 9.3 | Prefold in subunit |
| SBP | PF03110.5 | 25 | SBP domain |
| SET | PF00856.18 | 23.5 | SET domain |
| SRF-TF | PF00319.9 | 11 | SRF-type transcription factor (DNA-binding and |
| | | | dimerisation domain) |
| SWIRM | PF04433.7 | 25 | SWIRM domain |
| TCP | PF03634.4 | −38 | TCP family transcription factor |
| Tub | PF01167.8 | −98 | Tub family |
| WRKY | PF03106.6 | 25 | WRKY DNA-binding domain |
| YDG_SRA | PF02182.8 | 25 | YDG/SRA domain |
| ZZ | PF00569.8 | 14 | Zinc finger, ZZ type |
| bZIP_1 | PF00170.11 | 24.5 | bZIP transcription factor |
| bZIP_2 | PF07716.5 | 15 | Basic region leucine zipper |
| zf-B_box | PF00643.14 | 15.3 | B-box zinc finger |
| zf-C2H2 | PF00096.16 | 17.7 | Zinc finger, C2H2 type |
| zf-C3HC4 | PF00097.15 | 16 | Zinc finger, C3HC4 typc (RING finger) |
| zf-CCCH | PF00642.15 | 0 | Zinc finger C-x8-C-x5-C-x3-H type (and similar) |
| zf-Dof | PF02701.6 | 25 | Dof domain, zinc finger |
| zf-LSD1 | PF06943.3 | 25 | LSD1 zinc finger |

### Example 5

### Plasmid Construction for Transferring Recombinant DNA

This example illustrates the construction of plasmids for transferring recombinant DNA into the nucleus of a plant cell which can be regenerated into a transgenic crop plant of this invention. Primers for PCR amplification of protein coding nucleotides of recombinant DNA are designed at or near the start and stop codons of the coding sequence, in order to eliminate most of the 5′ and 3′ untranslated regions. DNA of interest, i.e. each DNA identified in Table 2 and the DNA for the identified homologous genes, are cloned and amplified by PCR prior to insertion into the insertion site the base vector.

### A. Plant Expression Constructs For Corn Transformation

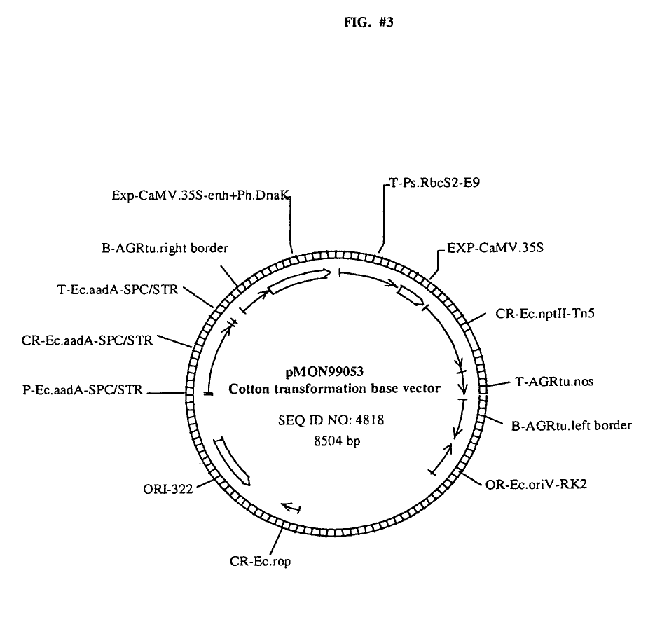

Elements of an exemplary common expression vector, pMON93039 are illustrated in Table 20. The exemplary base vector which is especially useful for corn transformation is illustrated in FIG. 2 and assembled using technology known in the art.

TABLE 20
pMON93039

| | | | Coordinates of |
|---|---|---|---|
| | | | SEQ ID NO: |
| function | name | annotation | 4816 |
| Agrobacterium | B-AGRtu.right | Agro right border | 11364-11720 |
| T-DNA | border | sequence, essential for | |
| trabsfer | | transfer of T-DNA. | |
| Gene of | E-Os.Act1 | upstream promoter | 19-775 |
| interest | | region of the rice actin | |
| expression | | 1 gene | |
| cassette | E- | duplicated35S A1-B3 | 788-1120 |
| | CaMV.35S.2xA1- | domain without TATA | |
| | B3 | box | |
| | P-Os.Act1 | promoter region of the | 1125-1204 |
| | | rice actin 1 gene | |
| | L-Ta.Lhcb1 | 5′ untranslated leader | 1210-1270 |
| | | of wheat major | |
| | | chlorophyll a/b binding | |
| | | protein | |
| | I-Os.Act1 | first intron and flanking | 1287-1766 |
| | | UTR exon sequences | |
| | | from the rice actin 1 | |
| | | gene | |
| | T-St.Pis4 | 3′ non-translated region | 1838-2780 |
| | | of the potato proteinase | |
| | | inhibitor II gene which | |
| | | functions to direct | |
| | | polyadenylation of the | |
| | | mRNA | |
| Plant | P-Os.Act1 | Promoter from the rice | 2830-3670 |
| selectable | | actin 1 gene | |
| marker | L-Os.Act1 | first exon of the rice | 3671-3750 |
| expression | | actin 1 gene | |
| cassette | I-Os.Act1 | first intron and flanking | 3751-4228 |
| | | UTR exon sequences | |
| | | from the rice actin 1 | |
| | | gene | |
| | TS-At.ShkG-CTP2 | Transit peptide region | 4238-4465 |
| | | of Arabidopsis EPSPS | |
| | CR-AGRtu.aroA- | coding region for | 4466-5833 |
| | CP4.nat | bacterial strain CP4 | |
| | | native arogA gene | |
| | T-AGRtu.nos | A 3′ non-translated | 5849-6101 |
| | | region of the nopa line | |
| | | synthase gene of | |
| | | Agrobacterium | |
| | | tumefaciens Ti plasmid | |
| | | which functions to | |
| | | direct polyadenylation | |
| | | of the mRNA. | |
| Agrobacterium | B-AGRtu.left | Agro left border | 6168-6609 |
| T-DNA | border | sequence, essential for | |
| transfer | | transfer of T-DNA. | |
| Maintenance | OR-Ec.oriV- | The vegetative origin | 6696-7092 |
| in E. coli | RK2 | of replication from | |
| | | plasmid RK2. | |
| | CR-Ec.rop | Coding region for | 8601-8792 |
| | | repressor of primer | |
| | | from the ColE1. | |
| | | plasmid. Expression of | |
| | | this gene product | |
| | | interferes with primer | |
| | | binding at the origin of | |
| | | replication, keeping | |
| | | plasmid copy number | |
| | | low. | |
| | OR-Ec.ori-ColE1 | The minimal origin of | 9220-9808 |
| | | replication from the E. | |
| | | coli plasmid ColE1. | |
| | P-Ec.aadA- | romoter for Tn7 | 10339-10380 |
| | SPC/STR | adenylyltransferase | |
| | | (AAD(3″)) | |
| | CR-Ec.aadA- | Coding region for Tn7 | 10381-11169 |
| | SPC/STR | adenylyltransferase | |
| | | (AAD(3″)) conferring | |
| | | spectinomycin and | |
| | | streptomycin | |
| | | resistance. | |
| | T-Ec.aadA- | 3′ UTR from the Tn7 | 11170-11227 |
| | SPC/STR | adenylyltransferase | |
| | | (AAD(3″)) gene of E. | |
| | | coli. | |

### B. Plant Expression Constructs for Soybean or Canola Transformation

Plasmids for use in transformation of soybean are also prepared. Elements of an exemplary common expression vector plasmid pMON82053 are shown in Table 21 below. This exemplary soybean transformation base vector illustrated in FIG. 2 is assembled using the technology known in the art. DNA of interest, i.e. each DNA identified in Table 2 and the DNA for the identified homologous genes, is cloned and amplified by PCR prior to insertion into the insertion site the base vector at the insertion site between the enhanced 35S CaMV promoter and the termination sequence of cotton E6 gene.

TABLE 21
pMON82053

| | | | Coordinates of |
|---|---|---|---|
| | | | SEQ ID NO: |
| function | name | annotation | 4817 |
| Agrobacterium | B-AGRtu.left | Agro left border sequence, essential | 6144-6585 |
| T-DNA | border | for transfer of T-DNA. | |
| transfer | | | |
| Plant | P-At.Act7 | Promoter from the arabidopsis actin | 6624-7861 |
| selectable | | 7 gene | |
| marker | L-At.Act7 | 5′UTR of Arabidopsis Act7 gene | |
| expression | I-At.Act7 | Intron from the Arabidopsis actin7 | |
| cassette | | gene | |
| | TS-At.ShkG- | Transit peptide region of Arabidopsis | 7864-8091 |
| | CTP2 | EPSPS | |
| | CR-AGRtu.aroA- | Synthetic CP4 coding region with | 8092-9459 |
| | CP4.nno_At | dicot preferred codon usage. | |
| | T-AGRtu.nos | A 3′ non-translated region of the | 9466-9718 |
| | | nopaline synthase gene of | |
| | | Agrobacterium tumefaciens Ti | |
| | | plasmid which functions to direct | |
| | | polyadenylation of the rnRNA. | |
| Gene of | P-CaMV.35S-enh | Promoter for 35S RNA from CaMV | 1-613 |
| interest | | containing a duplication of the −90 to | |
| expression | | −350 region. | |
| cassette | T-Gb.E6-3b | 3′ untranslated region from the fiber | 688-1002 |
| | | protein E6 gene of sea-island cotton; | |
| Agrobaterium | B-AGRtu.right | Agro right border sequence, essential | 1033-1389 |
| T-DNA | border | for transfer of T-DNA. | |
| transfer | | | |
| Maintenance | OR-Ec.oriV-RK2 | The vegetative origin of replication | 5661-6057 |
| in E. coli | | from plasmid RK2. | |
| | CR-Ec.rop | Coding region for repressor of | 3961-4152 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori-ColE1 | The minimal origin of replication | 2945-3533 |
| | | from the E. coli plasmid ColE1. | |
| | P-Ec.aadA- | romoter for Tn7 adenylyltransferase | 2373-2414 |
| | SPC/STR | (AAD(3″)) | |
| | CR-Ec.aadA- | Coding region for Tn7 | 1584-2372 |
| | SPC/STR | adenylyltransferase (AAD(3″)) | |
| | | conferring spectinomycin and | |
| | | streptomycin resistance. | |
| | T-Ec.aadA- | 3′ UTR from the Tn7 | 1526-1583 |
| | SPC/STR | adenylyltransferase (AAD(3″)) gene | |
| | | of E. coli. | |

### C. Plant Expression Constructs for Cotton Transformation

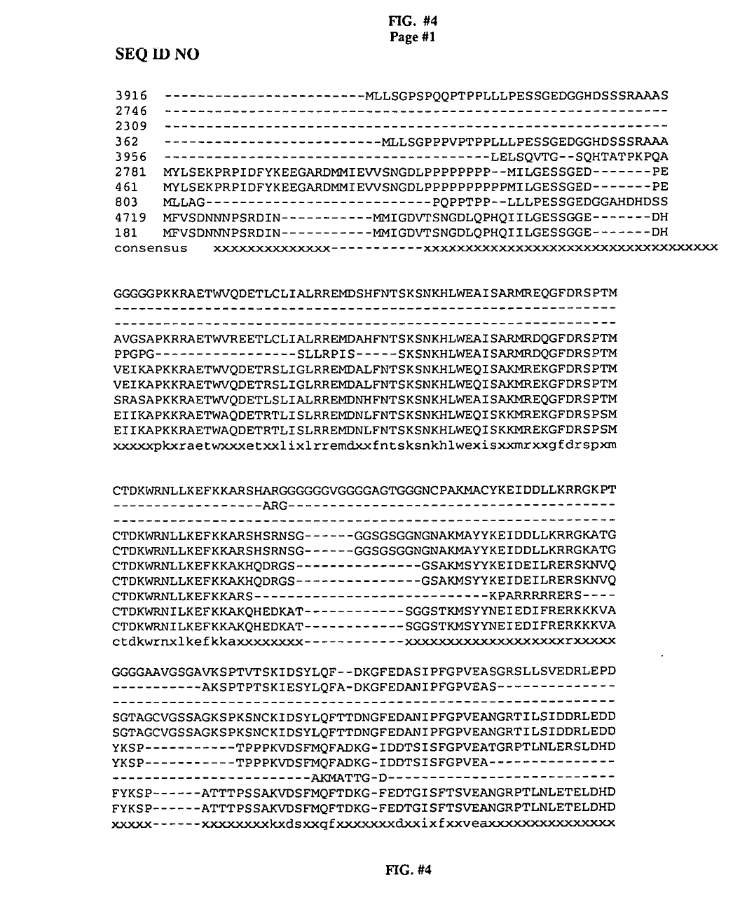

Plasmids for use in transformation of cotton are also prepared. Elements of an exemplary common expression vector plasmid pMON99053 are shown in Table 22 below and FIG. 3. Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 2 is amplified by PCR prior to insertion into the insertion site within the gene of interest expression cassette of one of the base.

TABLE 22
pMON99053

| | | | Coordinates |
|---|---|---|---|
| | | | of SEQ ID |
| function | name | annotation | NO: 4818 |
| Agrobacterium | B- | Agro right border sequence, | 1-357 |
| T-DNA transfer | AGRtu.right | essential for transfer of T- | |
| | border | DNA. | |
| Gene of interest | Exp- | Enhanced version of the 35S | 388-1091 |
| expression | CaMV.35S- | RNA promoter from CaMV | |
| cassette | enh + | plus the petunia hsp70 5′ | |
| | ph.DnaK | untranslated region | |
| | T-Ps.RbcS2- | The 3′ non-translated region of | 1165-1797 |
| | E9 | the pea RbcS2 gene which | |
| | | functions to direct | |
| | | polyadenylation of the mRNA. | |
| Plant selectable | Exp- | Promoter and 5′ untranslated | 1828-2151 |
| marker | CaMV.35S | region of the 35S RNA from | |
| expression | | CaMV | |
| cassette | CR-Ec.nptII- | Neomycin Phosphotransferase | 2185-2979 |
| | Tn5 | II gene that confers resistance | |
| | | to neomycin and kanamycin | |
| | T- | A 3′ non-translated region of | 3011-3263 |
| | AGRtu.nos | the nopaline synthase gene of | |
| | | Agrobacterium tumefaciens Ti | |
| | | plasmid which functions to | |
| | | direct polyadenylation of the | |
| | | mRNA. | |
| Agrobacterium | B- | Agro left border sequence, | 3309-3750 |
| T-DNA | AGRtu.left | essential for transfer of T- | |
| transfer | border | DNA. | |
| Maintenance | OR-Ec.oriV- | The vegetative origin of | 3837-4233 |
| in | RK2 | replication from plasmid RK2. | |
| E. coli | CR-Ec.rop | Coding region for repressor of | 5742-5933 |
| | | primer from the ColE1 | |
| | | plasmid. Expression of this | |
| | | gene product interferes with | |
| | | primer binding at the origin of | |
| | | replication, keeping plasmid | |
| | | copy number low. | |
| | OR-Ec.ori- | The minimal origin of | 6361-6949 |
| | ColE1 | replication from the E. coli | |
| | | plasmid ColE1. | |
| | P-Ec.aadA- | romoter for Tn7 | 7480-7521 |
| | SPC/STR | adenylyltransferase (AAD(3″)) | |
| | CR-Ec.aadA- | Coding region for Tn7 | 7522-8310 |
| | SPC/STR | adenylyltransferase (AAD(3″)) | |
| | | conferring spectinomycin and | |
| | | streptomycin resistance. | |
| | T-Ec.aadA- | 3′ UTR from the Tn7 | 8311-8368 |
| | SPC/STR | adenylyltransferase (AAD(3″)) | |
| | | gene of E. coli. | |

### Example 6

### Corn Plant Transformation

This example illustrates the production and identification of transgenic corn cells in seed of transgenic corn plants having an enhanced agronomic trait, i.e. enhanced nitrogen use efficiency, increased yield, enhanced water use efficiency, enhanced tolerance to cold and/or enhanced seed compositions as compared to control plants. Transgenic corn cells are prepared with recombinant DNA expressing each of the protein encoding DNAs listed in Table 2 by Agrobacterium-mediated transformation using the corn transformation constructs as disclosed in Example 5.

Corn transformation is effected using methods disclosed in U.S. Patent Application Publication 2004/0344075 A1 where corn embryos are inoculated and co-cultured with the Agrobacterium tumefaciens strain ABI and the corn transformation vector. To regenerate transgenic corn plants the transgenic callus resulting from transformation is placed on media to initiate shoot development in plantlets which are transferred to potting soil for initial growth in a growth chamber followed by a mist bench before transplanting to pots where plants are grown to maturity. The plants are self fertilized and seed is harvested for .screening as seed, seedlings or progeny R2 plants or hybrids, e.g., for yield trials in the screens indicated above.

Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and enhanced seed composition.

### Example 7

### Soybean Plant Transformation

This example illustrates the production and identification of transgenic soybean cells in seed of transgenic soybean plants having an enhanced agronomic trait, i.e. enhanced nitrogen use efficiency, increased yield, enhanced water use efficiency, enhanced tolerance to cold and/or enhanced seed compositions as compared to control plants. Transgenic soybean cells are prepared with recombinant DNA expressing each of the protein encoding DNAs listed in Table 1 by Agrobacterium-mediated transformation using the soybean transformation constructs disclosed in Example 5. Soybean transformation is effected using methods disclosed in U.S. Pat. No. 6,384,301 where soybean meristem explants arc wounded then inoculated and co-cultured with the soybean transformation vector, then transferred to selection media for 6-8 weeks to allow selection and growth of transgenic shoots.

The transformation is repeated for each of the protein encoding DNAs identified in Table 2. Transgenic shoots producing roots are transferred to the greenhouse and potted in soil. Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and enhanced seed composition.

### Example 8

### Selection of Transgenic Plants With Enhanced Agronomic Trait(s)

This example illustrates identification of nuclei of the invention by screening derived plants and seeds for an enhanced trait identified below.

Many transgenic events which survive to fertile transgenic plants that produce seeds and progeny plants will not exhibit an enhanced agronomic trait. Populations of transgenic seed and plants prepared in Examples 6 and 7 are screened to identify those transgenic events providing transgenic plant cells with a nucleus having recombinant DNA imparting an enhanced trait. Each population is screened for enhanced nitrogen use efficiency, increased yield, enhanced water use efficiency, enhanced tolerance to cold and heat, increased level of oil and protein in seed using assays described below. Plant cell nuclei having recombinant DNA with each of the genes identified in Table 2 and the identified homologs are identified in plants and seeds with at least one of the enhanced traits.

### A. Selection for Enhanced Nitrogen Use Efficiency

Transgenic corn plants with nuclei of the invention are planted in fields with three levels of nitrogen (N) fertilizer being applied, i.e. low level (0 pounds per acre N), medium level (80 pounds per acre N) and high level (180 pounds per acre N). Liquid 28% or 32% UAN (Urea, Ammonium Nitrogen) are used as the N source and apply by broadcast boom and incorporate with a field cultivator with rear rolling basket in the same direction as intended crop rows. Although there is no N applied in the low level treatment, the soil should still be disturbed in the same fashion as the treated area. Transgenic plants and control plants can be grouped by genotype and construct with controls arranged randomly within genotype blocks. For improved statistical analysis each type of transgenic plant can be tested by 3 replications and across 4 locations. Nitrogen levels in the fields are analyzed before planting by collecting sample soil cores from 0-24″ and 24 to 48″ soil layer. Soil samples are analyzed for nitrate-nitrogen, phosphorus (P), potassium (K), organic matter and pH to provide baseline values. P, K and micronutrients are applied based upon soil test recommendations.

Transgenic corn plants prepared in Example 6 and which exhibit a 2 to 5% yield increase as compared to control plants when grown in the high nitrogen field are selected as having nuclei of the invention. Transgenic corn plants which have at least the same or higher yield as compared to control plants when grown in the medium nitrogen field are selected as having nuclei of the invention. Transgenic corn plants having a nucleus with DNA identified in Table 3 as imparting nitrogen use efficiency (LN) and homologous DNA are selected from a nitrogen use efficiency screen as having a nucleus of this invention.

### B. Selection for Increased Yield

Many transgenic plants of this invention exhibit increased yield as compared to a control plant. Increased yield can result from enhanced seed sink potential, i.e. the number and size of endosperm cells or kernels and/or enhanced sink strength, i.e. the rate of starch biosynthesis. Sink potential can be established very early during kernel development, as endosperm cell number and size are determined within the first few days after pollination.

Much of the increase in corn yield of the past several decades has resulted from an increase in planting density. During that period, corn yield has been increasing at a rate of 2.1 bushels/acre/year, but the planting density has increased at a rate of 250 plants/acre/year. A characteristic of modern hybrid corn is the ability of these varieties to be planted at high density. Many studies have shown that a higher than current planting density should result in more biomass production, but current germplasm does not perform. well at these higher densities. One approach to increasing yield is to increase harvest index (HI), the proportion of biomass that is allocated to the kernel compared to total biomass, in high density plantings.

Effective yield selection of enhanced yielding transgenic corn events uses hybrid progeny of the transgenic event over multiple locations with plants grown under optimal production management practices, and maximum pest control. A useful target for increased yield is a 5% to 10% increase in yield as compared to yield produced by plants grown from seed for a control plant. Selection methods can be applied in multiple and diverse geographic locations, for example up to 16 or more locations, over one or more planting seasons, for example at least two planting seasons to statistically distinguish yield improvement from natural environmental effects. Each of the transgenic corn plants and soybean plants with a nucleus of the invention prepared in Examples 6 and 7 are screened for yield enhancement. At least one event from each of the corn plants is selected as having at least between 3 and 5% increase in yield as compared to a control plant as having a nucleus of this invention.

### C. Selection for Enhanced Water Use Efficiency (WUE)

The following is a high-throughput method for screening for water use efficiency in a greenhouse to identify the transgenic corn plants with a nucleus of this invention. This selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment. The hydration status of the shoot tissues following the drought is also measured. The plant heights are measured at three time points. The first is taken just prior to the onset drought when the plant is 11 days old, which is the shoot initial height (SIH). The plant height is also measured halfway throughout the drought/re-water regimen, on day 18 after planting, to give rise to the shoot mid-drought height (SMH). Upon the completion of the final drought cycle on day 26 after planting, the shoot portion of the plant is harvested and measured for a final height, which is the shoot wilt height (SWH) and also measured for shoot wilted biomass (SWM). The shoot is placed in water at 40 degree Celsius in the dark. Three days later, the shoot is weighted to give rise to the shoot turgid weight (STM). After drying in an oven for four days, the shoots are weighted for shoot dry biomass (SDM). The shoot average height (SAH) is the mean plant height across the 3 height measurements. The procedure described above can be adjusted for +/−˜one day for each step given the situation.

To correct for slight differences between plants, a size corrected growth value is derived from SIH and SWH. This is the Relative Growth Rate (RGR). Relative Growth Rate (RGR) is calculated for each shoot using the formula [RGR%=(SWH−SIH)/((SWH+SIH)/2)×100]. Relative water content (RWC) is a measurement of how much (%) of the plant was water at harvest. Water Content (RWC) is calculated for each shoot using the formula [RWC%=(SWM−SDM)/(STM−SDM)×100]. Fully watered corn plants of this age run around 98% RWC.

Transgenic corn plants and soybean plants prepared in Examples 6 and 7 are screened for water use efficiency. Transgenic plants having at least a 1% increase in RGR and RWC as compared to control plants are identified as having enhanced water used efficiency and are selected as having a nucleus of this invention. Transgenic corn and soybean plants having in their nucleus DNA identified in Table 3 as imparting drought tolerance enhancement (DS, HS, SS, and PEG) and homologous DNA are identified as showing increased water use efficiency as compared to control plants and are selected as having a nucleus of this invention.

### D. Selection for Growth Under Cold Stress

Cold germination assay—Three sets of seeds are used for the assay. The first set consists of positive transgenic events (F1 hybrid) where the genes of the present invention are expressed in the seed. The second seed set is nontransgenic, wild-type negative control made from the same genotype as the transgenic events. The third set consisted of two cold tolerant and one cold sensitive commercial check lines of corn. All seeds are treated with a fungicide “ Captan” (MAESTRO® 80DF Fungicide, Arvesta Corporation, San Francisco, Calif., USA). 0.43 mL Captan is applied per 45 g of corn seeds by mixing it well and drying the fungicide prior to the demonstration.

Corn kernels are placed embryo side down on blotter paper within an individual cell (8.9×8.9 cm) of a germination tray (54×36 cm). Ten seeds from an event are placed into one cell of the germination tray. Each tray can hold 21 transgenic events and 3 replicates of wildtype (LH244SDms+LH59), which is randomized in a complete block design. For every event there are five replications (five trays). The trays are placed at 9.7C for 24 days (no light) in a Convrion® growth chamber (Conviron Model PGV36, Controlled Environments, Winnipeg, Canada). Two hundred and fifty millilters of deionized water are added to each germination tray. Germination counts are taken 10th, 11th, 12th, 13th, 14th, 17th, 19th, 21st, and 24th day after start date of the demonstration. Seeds are considered germinated if the emerged radicle size is 1 cm. From the germination counts germination index is calculated.

The germination index is calculated as per:

Germination index=(Σ([T+1−nᵢ]*[Pᵢ−Pᵢ₋₁]))/T

where T is the total number of days for which the germination assay is performed. The number of days after planting is defined by n. “i” indicated the number of times the germination had been counted, including the current day. P is the percentage of seeds germinated during any given rating. Statistical differences are calculated between transgenic events and wild type control. After statistical analysis, the events that show a statistical significance at the p level of less than 0.1 relative to wild-type controls will advance to a secondary cold selection. The secondary cold screen is conducted in the same manner of the primary selection only increasing the number of repetitions to ten. Statistical analysis of the data from the secondary selection is conducted to identify the events that show a statistical significance at the p level of less than 0.05 relative to wild-type controls.

Transgenic corn plants and soybean plants prepared in Examples 6 and 7 are screened for water use efficiency. Transgenic plants having at least a 5% increase in germination index as compared to control plants are identified as having enhanced cold stress tolerance and are selected as having a nucleus of this invention. Transgenic corn and soybean plants having in their nucleus DNA identified in Table 3 as imparting cold tolerance enhancement (CK or CS) and homologous DNA are identified as showing increased cold stress tolerance as compared to control plants and are selected as having a nucleus of this invention.

E. Screens for Transgenic Plant Seeds With Increased Protein and/or Oil Levels

The following is a high-throughput selection method for identifying plant seeds with improvement in seed composition using the Infratec® 1200 series Grain Analyzer, which is a near-infrared transmittance spectrometer used to determine the composition of a bulk seed sample. Near infrared analysis is a non-destructive, high-throughput method that can analyze multiple traits in a single sample scan. An NIR calibration for the analytes of interest is used to predict the values of an unknown sample. The NIR spectrum is obtained for the sample and compared to the calibration using a complex chemometric software package that provides a predicted values as well as information on how well the sample fits in the calibration.

Infratec® Model 1221, 1225, or 1227 analyzer with transport module by Foss North America is used with cuvette, item #1000-4033, Foss North America or for small samples with small cell cuvette, Foss standard cuvette modified by Leon Girard Co. Corn and soy check samples of varying composition maintained in check cell cuvettes are supplied by Leon Girard Co. NIT collection software is provided by Maximum Consulting Inc. Calculations are performed automatically by the software. Seed samples are received in packets or containers with barcode labels from the customer. The seed is poured into the cuvettes and analyzed as received.

TABLE 23

| Typical sample(s): | Whole grain corn and soybean seeds |
|---|---|
| Analytical time to run | Less than 0.75 min per sample |
| method: | |
| Total elapsed time per | 1.5 minute per sample |
| run: | |
| Typical and minimum | Corn typical: 50 cc; minimum 30 cc |
| sample size: | Soybean typical: 50 cc; minimum 5 cc |
| Typical analytical | Determined in part by the specific calibration. |
| range: | Corn-moisture 5-15%, oil 5-20%, protein 5-30%, |
| | starch 50-75%, and density 1.0-1.3%. |
| | Soybean-moisture 5-15%, oil 15-25%, and |
| | protein 35-50%. |

Transgenic corn plants and soybean plants prepared in Examples 6 and 7 are screened for increased protein and oil in seed. Transgenic inbred corn and soybean plants having an increase of at least 1 percentage point in the total percent seed protein or at least 0.3 percentage point in total seed oil and transgenic hybrid corn plants having an increase of at least 0.4 percentage point in the total percent seed protein as compared to control plants are identified as having enhanced seed protein or enhanced seed oil and are selected as having a nucleus of this invention.

### Example 9

### Cotton Transgenic Plants With Enhanced Agronomic Traits

Cotton transformation is performed as generally described in WO0036911 and in U.S. Pat. No. 5,846,797. Transgenic cotton plants containing each of the recombinant DNA having a sequence of SEQ ID NO: 1 through SEQ ID NO: 114 are obtained by transforming with recombinant DNA from each of the genes identified in Table 1. Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA. The specified culture conditions are growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

The transgenic cotton plants of this invention are identified from among the transgenic cotton plants by agronomic trait screening as having increased yield and enhanced water use efficiency.

### Example 10

### Canola Plants With Enhanced Agrominic Traits

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with a suspension of overnight grown Agrobacterium containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterizations are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant

Transgenic canola plant cells are transformed with recombinant DNA from each of the genes identified in Table 2. Transgenic progeny plants and seed of the transformed plant cells are screened for enhanced water use efficiency, enhanced cold tolerance, increased yield, enhanced nitrogen use efficiency, enhanced seed protein and enhanced seed oil.

### Example 11

### Monocot and Dicot Plant Transformation for the Suppression of Endogeneous Protein

This example illustrates monocot and dicot plant transformation to produce nuclei of this invention in cells of a transgenic plant by transformation where the recombinant DNA suppresses the expression of an endogenous protein identified in Table 24.

Corn callus and soybean tissue are transformed as describe in Examples 6 and 7 using recombinant DNA in the nucleus with DNA that transcribes to RNA that forms double-stranded RNA targeted to an endogenous gene with DNA encoding the protein. The genes for which the double-stranded RNAs are targeted are the native gene in corn and soybean that are homolog of the genes encoding the protein of Arabidopsis thaliana as identified in table 24.

Populations of transgenic plants prepared in Examples 6, 7 or 10 with DNA for suppressing a gene identified in Table 3 as providing an enhanced trait by gene suppression are screened to identify an event from those plants with a nucleus of the invention by selecting the trait identified in this specification.

TABLE 24

| PEP | | | | |
|---|---|---|---|---|
| SEQ | | Construct | | |
| ID | Pfam module | ID | Traits | |
| 115 | AP2 | 10177 | LN | |
| 116 | zf-C2H2 | 12155 | CS | SS |
| 118 | F-box::Tub | 11873 | LN | PEG |
| 154 | Pex2_Pex12::zf-C3HC4 | 11113 | CS | |
| 188 | Myb_DNA-binding::Myb_DNA- | 12325 | LN | |
| | binding | | | |
| 200 | bZIP_1 | 71228 | CK | |

What is claimed is:

1. A plant cell nucleus with stably-integrated, recombinant DNA comprising a promoter that is functional in plant cells and that is operably linked to protein coding DNA encoding a protein having an amino acid sequence comprising a Pfam domain module zf-CCCH::KII_1:zf-CCCH;
wherein said plant cell nucleus is selected by screening a population of transgenic plants that have said recombinant DNA in its nuclei and express said protein for an enhanced trait as compared to control plants that do not have said recombinant DNA in their nuclei; and
wherein said enhanced trait is selected from group of enhanced traits consisting of enhanced water use efficiency, enhanced cold tolerance, increased yield, enhanced nitrogen use efficiency, enhanced seed protein and enhanced seed oil.

2. A plant cell nucleus with stably integrated, recombinant DNA, wherein
a. said recombinant DNA comprises a promoter that is functional in said plant cell and that is operably linked to a protein coding DNA encoding a protein having an amino acid sequence comprising a Pfam domain module selected from the group consisting of AP2, zf-C2H2, PLATZ, F-box::Tub, zf-C3HC4::YDG_SRA::zf-C3HC4, SBP, HLH, AP2, zf-B_box::zf-B_box, zf-C3HC4, AP2, HMG_box::HMG_box::HMG_box, zf-C3HC4, zf-C2H2, GATA, HLH, AP2, NAM, zf-Dof, WRKY, AP2, HMG_box, zf-CCCH::KH_1::zf-CCCH, SRF-TF, WRKY, zf-C3HC4, zf-Dof, zf-Dof, AP2, AP2, DUF248, zf-C2H2, SRF-TF::K-box, zf-Dof, zf-C2H2, Myb_DNA-binding:: Myb_DNA-binding, Myb_DNA-binding::Myb_DNA-binding::Myb_DNA-binding, SRF-TF, Pex2_Pex12::zf-C3HC4, bZIP_2, HLH, GRAS, Myb_DNA-binding, GRAS, F-box, GRAS, WRKY, AT_hook::DUF296, GRAS, Myb_DNA-binding:: Myb_DNA-binding, Myb_DNA-binding::Myb_DNA-binding, HLH, zf-C2H2, NAM, zf-B_box::zf-B_box, Myb_DNA-binding, NAM, PHD, SRF-TF::K-box, zf-C3HC4, HLH, SRF-TF::K-box, Myb_DNA-binding, WRKY, SRF-TF::K-box, Myb_DNA-binding, Myb_DNA-binding::Linker_histone, Myb_DNA-binding::Myb_DNA-binding, Myb_DNA-binding::Myb_DNA-binding, bZIP_1, Myb_DNA-binding::Myb_DNA-binding, SRF-TF::K-box, SRF-TF::K-box, CBFD_NFYB_HMF, AUX_IAA, Myb_DNA-binding::Myb_{—}DNA-binding, LIM::LIM, IBR, SET, bZIP_1, Mov34, ZZ::Myb_DNA-binding::SWIRM, bZIP_1, E2F_TDP::E2F_TDP, Myb_DNA-binding, Prefoldin, NAM, HD-ZIP_N::Homeobox::HALZ, Myb_DNA-binding::Myb_DNA-binding, AP2, GATA, zf-C3HC4, SRF-TF::K-box, bZIP_1, TCP, zf-C3HC4, Ank::Ank::zf-CCCH::zf-CCCH, Myb_DNA-binding::Myb_DNA-binding, TCP, AP2, ZZ, zf-C2H2::zf-C2H2, AP2, SRF-TF::K-box, B3, zf-LSD1::zf-LSDI, and HLH;
b. said recombinant DNA comprises a promoter that is functional in said plant cell and that is operably linked to a protein coding DNA encoding a protein comprising an amino acid sequence with at least 90% identity to a consensus amino acid sequence selected from the group consisting of SEQ ID NO: 4819 through 4825; or
c. said recombinant DNA suppresses comprises a promoter that is functional in said plant cell and operably linked to DNA that transcribe into RNA that suppresses the level of an endogenous protein wherein said endogenous protein has an amino acid sequence comprising a pfam domain module selected from the group consisting of AP2, zf-C2H2, F-box::Tub, Pex2_Pex12::zf-C3HC4, Myb_DNA-binding::Myb_DNA-binding, and bZIP_1; and wherein said plant cell nucleus is selected by screening a population of transgenic plants that have said recombinant DNA and an enhanced trait as compared to control plants that do not have said recombinant DNA in their nuclei; and wherein said enhanced trait is selected from group of enhanced traits consisting of enhanced water use efficiency, enhanced cold tolerance, enhanced heat tolerance, enhanced resistance to salt exposure, enhanced shade tolerance, increased yield, enhanced nitrogen use efficiency, enhanced seed protein and enhanced seed oil.

3. The plant cell nucleus of claim 2 wherein said protein coding DNA encodes a protein having an amino acid sequence selected from the group consisting of SEQ ID NO: 115 through SEQ ID NO: 4815.

4. The plant cell nucleus of claim 2 further comprising DNA expressing a protein that provides tolerance from exposure to an herbicide applied at levels that are lethal to a wild type of said plant cell.

5. The plant cell nucleus of claim 4 wherein said herbicide is a glyphosate, dicamba, or glufosinate compound.

6. A transgenic plant cell or plant comprising a plurality of plant cells with the plant cell nucleus of claim 2.

7. The transgenic plant cell or plant of claim 6 which is homozygous for said recombinant DNA.

8. A transgenic seed comprising a plurality of plant cells with a plant cell nucleus of claim 2.

9. The transgenic seed of claim 8 from a corn, soybean, cotton, canola, alfalfa, wheat or rice plant.

10. A transgenic pollen grain comprising a haploid derivative of a plant cell nucleus of claim 2.

11. A method for manufacturing non-natural, transgenic seed that can be used to produce a crop of transgenic plants with an enhanced trait resulting from expression of recombinant DNA in a nucleus of claim 2, wherein said method for manufacturing said transgenic seed comprising:
(a) screening a population of plants for said enhanced trait and said recombinant DNA, wherein individual plants in said population can exhibit said trait at a level less than, essentially the same as or greater than the level that said trait is exhibited in control plants which do not contain the recombinant DNA, wherein said enhanced trait is selected from the group of enhanced traits consisting of enhanced water use efficiency, enhanced cold tolerance, enhanced heat tolerance, enhanced high salinity tolerance, enhanced shade tolerance, increased yield, enhanced nitrogen use efficiency, enhanced seed protein and enhanced seed oil,
(b) selecting from said population one or more plants that exhibit said trait at a level greater than the level that said trait is exhibited in control plants, and
(c) collecting seeds from selected plant selected from step b.

12. The method of claim 11 wherein said method for manufacturing said transgenic seed further comprising
(a) verifying that said recombinant DNA is stably integrated in said selected plants, and
(b) analyzing tissue of said selected plant to determine the expression or suppression of a protein having the function of a protein having an amino acid sequence selected from the group consisting of SEQ ID NO:115-228.

13. The method of claim 11 wherein said seed is corn, soybean, cotton, alfalfa, canola wheat or rice seed.

14. A method of producing hybrid corn seed comprising:
(a) acquiring hybrid corn seed from a herbicide tolerant corn plant which also has stably-integrated, recombinant DNA in a nucleus of claim 2;
(b) producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA;
(c) selecting corn plants which are homozygous and hemizygous for said recombinant DNA by treating with an herbicide;
(d) collecting seed from herbicide-treated-surviving corn plants and planting said seed to produce further progeny corn plants;
(e) repeating steps (c) and (d) at least once to produce an inbred corn line; and
(f) crossing said inbred corn line with a second corn line to produce hybrid seed.
